(19) United States
(12) Patent Application Publication
(10) Pub. No.: US 2026/0062683 A1
(43) Pub. Date: Mar. 5, 2026

(54) KAURENOIC ACID 13-HYDROXYLASE (KAH) VARIANTS AND USES THEREOF

(71) Applicant: Amyris, Inc., Emeryville, CA (US)

(72) Inventors: Andrew P. KLEIN, Emeryville, CA (US); Sean LUND, Emeryville, CA (US); Gale WICHMANN, Emeryville, CA (US); Irina KORYAKINA, Emeryville, CA (US); Kyle HOGAN, Emeryville, CA (US); Svetlana BORISOVA, Emeryville, CA (US)

(21) Appl. No.: 19/254,544

(22) Filed: Jun. 30, 2025

Related U.S. Application Data
(63) Continuation of application No. 17/608,437, now Pat. No. 12,467,039.
(60) Provisional application No. 62/842,810, filed on May 3, 2019.

(51) Int. Cl. C12N 9/02; C12P 19/56

(52) U.S. Cl. CPC C12N 9/0073; C12P 19/56

(57) ABSTRACT

Provided herein are genetically modified host cells, compositions, and methods for improved production of steviol glycosides. The host cells are genetically modified to contain a heterologous nucleic acid that expresses novel and optimized variants of Ro.KAH. The host cell further contains one or more heterologous nucleotide sequence encoding further enzymes of a pathway capable of producing one or more steviol glycosides in the host cell. The host cells, compositions, and methods described herein provide an efficient route for the heterologous production of steviol glycosides including rebaudioside A, rebaudioside D, and rebaudioside M.

### SEQUENCE LISTING

The instant application contains a Sequence Listing which has been submitted electronically in XML format and is hereby incorporated by reference in its entirety. Said XML copy, created on Jun. 26, 2025, is named 51494-009003_Sequence_Listing_6_26_25 and is 33,427 bytes in size.

### FIELD OF THE INVENTION

The present disclosure relates to Kaurenoic Acid 13-Hydroxylase (KAH) variants, host cells comprising the same, and methods of their use for the production of heterologous molecules.

### BACKGROUND

Reduced-calorie sweeteners derived from natural sources are desired to limit the health effects of high-sugar consumption. The stevia plant (Stevia rebaudiana Bertoni) produces a variety of sweet-tasting glycosylated diterpenes termed steviol glycosides. Of all the known steviol glycosides, Reb M has the highest potency (˜300 times sweeter than sucrose) and has the most appealing flavor profile. However, Reb M is only produced in minute quantities by the stevia plant and is a small fraction of the total steviol glycoside content (<1.0%), making the isolation of Reb M from stevia leaves impractical. Alternative methods of obtaining Reb M are needed. One such approach is the application of synthetic biology to design microorganisms (e.g. yeast) that produce large quantities of Reb M from sustainable feedstock sources.

To economically produce a product using synthetic biology, each step in the bioconversion from feedstock to product needs to have a high conversion efficiency (ideally >90%). In our engineering of yeast to produce Reb M, we noted that particular enzymatic steps performed poorly in yeast when the wild-type enzyme was used. In order to increase the productivity and yield of Reb M in microorganisms, we sought to produce variant enzymes that performed better than wild-type enzymes. One such enzyme is kaurenoic acid 13-hydroxylase (KAH) which catalyzes the conversion of ent-kaurenoic acid into steviol.

### SUMMARY OF THE INVENTION

Provided herein are compositions and methods for the improved conversion of kaurenoic acid to steviol. These compositions and methods are based in part on the production of certain variant kaurenoic acid hydroxylases (KAHs) that are capable of converting kaurenoic acid to steviol with high efficiency.

In one aspect, the invention provides for a host cell having a kaurenoic acid hydroxylase polypeptide having at least 90% sequence identity to the sequence of SEQ ID NO: 1.

In one aspect, the invention provides generally for a variant kaurenoic acid hydroxylase polypeptide having the amino acid sequence of SEQ ID NO: 1, wherein the sequence further contains one or more amino acid substitutions.

In another aspect, the invention provides for nucleic acids encoding variant kaurenoic acid hydroxylase polypeptides having the amino acid sequence of SEQ ID NO: 1 wherein the sequence further contains one or more amino acid substitutions. In another aspect, the invention provides for nucleic acids encoding any one of the variant polypeptides described herein.

In yet another aspect, the invention provides for host cells containing the variant kaurenoic acid hydroxylase polypeptides having the amino acid sequence of SEQ ID NO: 1, wherein the sequence further contains one or more amino acid substitutions. In another aspect, the invention provides for a host cell comprising any one of the polypeptides or nucleic acids described herein.

In a further aspect, the invention provides for host cells containing nucleic acids encoding variant kaurenoic acid hydroxylase polypeptides having the amino acid sequence of SEQ ID NO: 1, wherein the sequence further contains one or more amino acid substitutions.

In an embodiment, the invention provides that the one or more amino acid substitutions are selected from K69R, V343G, T403V, H491P, P72D, L64D, Q84C, L64G, E206D, Y238C, A210G, L64N, I237C, L11V, N207F, M73G, W8G, E60R, Y55S, N475G, D292P, P161C, K267D, L485F, A396F, R507A, P72T, I132G, N61P, K119V, T220E, P72G, Q513R, S133G, Y506V, K69P, E60G, K224C, M73H, H379G, P72C, K314P, W202A, G466F, N49A, S339G, N160D, T216G, D102Y, F246G, M58P, T220R, R458D, M58G, A68I, S70P, F88V, T240D, L205I, K167G, L232M, S62R, G56D, Q244G, A242D, N49R, Q513G, W29T, L303D, T378D, I508L, W202Q, S505R, R233C, I104D, M258G, K69G, F88D, F88S, A217V, E230C, R507G, G466S, G56S, E230G, Y55G, A503C, S460I, I129R, S245G, F246S, Q84L, S133R, T509V, R507E, R233T, V30F, A68G, G56N, T162G, A68P, S165D, K119Y, W29C, S165P, W29V, I284G, A217L, Q335V, L65S, F53R, Y55P, W202V, K224V, W29A, H164G, Q244D, K291C, L65G, K167S, C327I, K291S, D57G, K167H, N160T, W202C, A242G, F88R, I104N, G466D, N475D, K119S, T123D, T216A, S339A, P161D, I104R, L54G, M171F, L232Y, D293C, V340A, T162A, A297V, I104H, F332L, A236R, K224I, S452D, I104A, V340S, F229Y, A297Y, and A297F. In another embodiment, the one or more amino acid substitutions are selected from N146W, A297Y, A236S, V9S, G466F, T283D, T142G, T425V, L459C, T283A, T283G, S460V, S133G, I129V, W52G, S505I, I243T, V340S, S460C, S452D, L118I, S505V, T123D, W52C, S460I, S457G, W52R, W52N, N146T, G466A, and W52T. In a particular embodiment, the one or more amino acid substitutions are selected from A297V, I104H, F332L, A236R, K224I, S452D, I104A, I104A, V340S, F229Y, A297Y, and A297F. In a preferred embodiment, the one or more amino acid substitutions are selected from S452D, I104A, V340S, F229Y, A297Y, and A297F. In further embodiments, the variant polypeptide has a substitution of the N-terminal signal sequence with the signal sequence of another p450 polypeptide. In some embodiments, the N-terminal signal sequence corresponds to amino acids 1-25 of SEQ ID NO: 1. In some embodiments, the signal sequence of another p450 polypeptide has the amino acid sequence of SEQ ID NO: 22. In some embodiments, the variant polypeptide includes the amino acid substitution A297Y and may also include one or more additional amino acid substitutions. In some embodiments, the variant polypeptide includes the amino acid substitutions comprising N146T/A297Y and may also include one or more additional amino acid substitutions. In some embodiments, the variant polypeptide has the amino acid substitutions selected from N146T/A297Y/G466A; W52T/N146T/A297Y; T142G/N146T/A297Y/G466A; W52T/T142G/N146T/A297Y; and W52T/T142G/N146T/A297Y/G466A. In preferred embodiments, the variant polypeptide has the amino acid sequence of SEQ ID NO: 17, SEQ ID NO: 18, SEQ ID NO: 19, SEQ ID NO: 20, and SEQ ID NO: 21. In some embodiments, the variant polypeptide has the amino acids substitutions selected from W52T/T142G/G466A; A145G/N14F/A297Y; N146W/A297Y/S460I; W52N/N146W/A297Y; F332L/S452D; N146W/A297Y; and Q84R/N146T/A297Y.

In an embodiment, of the invention the host cell is capable of producing one or more steviol glycosides. In another embodiment, the one or more steviol glycosides are selected from RebA, RebB, RebD, RebE, and RebM. In a preferred embodiment, the one or more steviol glycosides is RebM.

In an embodiment, the host cells of the invention contain one or more nucleic acids encoding one or more enzymes of a pathway for making a steviol glycoside. In another embodiment the host cells contain a nucleic acid encoding a geranylgeranyl diphosphate synthase. In yet another embodiment, the host cells contain a nucleic acid encoding a copalyl diphosphate synthase. In another embodiment, the host cells contain a nucleic acid encoding an ent-kaurene synthase. In yet another embodiment, the host cells contain a nucleic acid encoding a kaurene oxidase. In another embodiment, the host cells contain a nucleic acid encoding a cytochrome P450 reductase. In yet another embodiment, the host cells contain a nucleic acid encoding one or more uridine 5′-diphosphate-dependent glycosyltransferases. In a preferred embodiment, the host cells contain one or more nucleic acids encoding a geranylgeranyl diphosphate synthase, a copalyl diphosphate synthase, an ent-kaurene synthase, a kaurene oxidase, a cytochrome P450 reductase, UGT40087, UGT74G1, UGT76G1, UGT85C2, EUGT11, and UGT91D.

In an embodiment, the host cells can be bacterial cells, yeast cells, algal cells, insect cells, or plant cells. In a particular embodiment, the host cells are yeast cells. In a preferred embodiment, the host cells are Saccharomyces cerevisiae.

In yet another aspect, the invention provides for a method for producing one or more steviol glycosides involving the steps of a) culturing a population of host cells of the invention in a medium with a carbon source under conditions suitable for making one or more steviol glycosides to yield a culture broth; and b) recovering the one or more steviol glycosides from the culture broth. In a preferred embodiment, the method involves the recovery of RebM.

In a further embodiment, the invention provides for a fermentation composition containing a host cell comprising a nucleic acid encoding a variant kaurenoic acid hydroxylase of the invention; and one or more steviol glycosides produced by the host cell. In a preferred embodiment, the fermentation composition contains RebM.

In an embodiment, the kaurenoic acid hydroxylase polypeptide has an amino acid sequence at least 90% (e.g., at least 91%, 92%, 93%, 94%, 95%, 96%, 97%, 98%, or 99%) identical to the sequence of SEQ ID NO: 1. In a preferred embodiment, host cells of the invention have a kaurenoic acid hydroxylase polypeptide having the amino acid sequence of SEQ ID NO: 1. In some embodiments, host cells of the invention have a kaurenoic acid hydroxylase polypeptide having the amino acid sequence of the kaurenoic acid hydroxylase polypeptide comprises the amino sequence selected from SEQ ID NO: 1, SEQ ID NO: 17, SEQ ID NO: 18, SEQ ID NO: 19, SEQ ID NO: 20, and SEQ ID NO: 21.

In one aspect, the invention provides compositions and food that contains RebM produced by a method of the invention or by a host cell of the invention. In another aspect, the invention provides a composition containing a steviol glycoside (e.g., RebA, RebB, RebD, RebE, or RebM) that is free of any other plant-derived material. In some embodiments, the steviol glycoside is RebM.

### BRIEF DESCRIPTION OF THE FIGURES

FIG. 1 is a schematic showing an enzymatic pathway from the native yeast metabolite farnesyl pyrophosphate (FPP) to rebaudioside M (Reb M).

FIG. 2 is a schematic of the landing pad DNA construct used to insert KAH genes into Reb M strains. The landing pad consists of 500 bp of locus-targeting DNA sequences on either end of the construct to the genomic region upstream and downstream of the yeast locus of choice. The locus is chosen so that insertion of the landing pad does not delete any gene. Internally, the landing pad contains a GAL promoter followed by a recognition site for the F-CphI endonuclease and the yeast terminator. Endonuclease F-CphI cuts the recognition sequence creating a double strand break at the landing pad thus facilitating homologous recombination of the Ro.KAH DNA variants at the site.

FIG. 3 is a graph of total steviol glycosides (measured in μM) in whole cell broth relative to a Rs.KAH control. Yeast strains with different overexpressed KAH genes were grown in microtiter plates. Also shown are the data for the parent strain that does not contain any KAH. Dark vertical lines represent 95% confidence interval of the mean (N=8).

FIG. 4 is a graph of total steviol glycosides (measured in μM) in whole cell broth of Reb M producing yeast containing mutants of Ro.KAH generated in a site saturation mutagenesis library relative to wild-type Ro.KAH-containing yeast. Yeast strains with different overexpressed KAH genes were grown in microtiter plates. Also shown are the data for the parent strain that does not contain any KAH and yeast strains containing wild-type Ro.KAH and Sr.KAH.

FIG. 5 is a graph of total steviol glycosides (measured in μM) in whole cell broth of Reb M producing yeast containing mutants of Ro.KAH generated in combinatorial libraries relative to wild-type Ro.KAH-containing yeast (also shown). Yeast strains with different overexpressed KAH genes were grown in microtiter plates.

FIG. 6A-6D depicts the proposed structure of the impurity C20H32O4+1Glc derived from a side-product of Ro.KAH activity on kaurenoic acid (FIG. 6A) and experimental evidence supporting impurity identification (FIG. 6B-6D). FIG. 6B is a region of interest from CAD chromatogram for whole cell broth extracts of yeast cultures expressing wild-type Ro.KAH, Sr.KAH, Rs.KAH, or no KAH. The peaks for Reb M and C20H32O4+1Glc are labeled. FIG. 6C is full mass spectrum for the peak at 9.5 min in the Ro.KAH chromatogram. FIG. 6D is an MS2 fragmentation spectra for the peak at 9.5 min in the Ro.KAH chromatogram.

FIG. 7 is a graph depicting improvements in the in vivo activity and product specificity of Ro.KAH mutants generated via site-directed saturation mutagenesis of Ro.KAH A297Y relative to wild-type Ro.KAH. Improvements in activity (y axis) calculated as follows,

$$\frac{{[ Reb M titer ]}_{mutant}}{{[ Reb M titer ]}_{wild - type}} .$$

Improvements in specificity (x axis) are calculated as follows,

$$\frac{{[ Reb M titer ]}_{mutant} {/ [ Reb M titer ]}_{wild - type}}{\begin{matrix}{[ C 20 H 32 O 4 + 1 Glc peak area ]}_{mutant} / \\ {[ C 20 H 32 O 4 + 1 Glc peak area ]}_{wild - type}\end{matrix}}$$

FIG. 8 is a graph depicting improvements in the in vivo activity and product specificity of Ro.KAH mutants generated via combinatorial site-directed saturation mutagenesis of Ro.KAH A297Y relative to wild-type Ro.KAH. Improvements in activity (y axis) and specificity (x axis) are calculated as described in FIG. 7 legend.

FIG. 9 is a graph depicting improvements in the in vivo activity and product specificity of Ro.KAH mutants generated via full site-directed saturation mutagenesis of Ro.KAH N146W/A297Y relative to Ro.KAH N146W/A297Y. Improvements in activity (y axis) and specificity (x axis) are calculated as described in FIG. 7 legend.

### DETAILED DESCRIPTION OF THE EMBODIMENTS

As used herein, the term “heterologous” refers to what is not normally found in nature. The term “heterologous nucleotide sequence” refers to a nucleotide sequence not normally found in a given cell in nature. As such, a heterologous nucleotide sequence may be: (a) foreign to its host cell (i.e., is “exogenous” to the cell); (b) naturally found in the host cell (i.e., “endogenous”) but present at an unnatural quantity in the cell (i.e., greater or lesser quantity than naturally found in the host cell); or (c) be naturally found in the host cell but positioned outside of its natural locus.

As used herein, the terms “native” or “endogenous” with reference to molecules, and in particular polypeptides and polynucleotides, indicate molecules that are expressed in the organism in which they originated or are found in nature. It is understood that expression of native polypeptides or polynucleotides may be modified in recombinant organisms.

As used herein, the term “variant” refers to molecules, and in particular polypeptides and polynucleotides, that differ from a specifically recited “reference” molecule in either structure or sequence. In preferred embodiments, the reference is a wild-type molecule. With respect to polypeptides and polynucleotides, variants refer to substitutions, additions, or deletions of the amino acid or nucleotide sequences respectively.

As used herein, the term “heterologous nucleic acid expression cassette” refers to a nucleic acid sequence that comprises a coding sequence operably linked to one or more regulatory elements sufficient to express the coding sequence in a host cell.

As used herein, the term “kaurenoic acid 13-hydroxylase” or “KAH” refers to an enzyme that is able to catalyze the conversion of ent-kaurenoic acid to steviol.

As used herein, the term “parent cell” refers to a cell that has an identical genetic background as a genetically modified host cell disclosed herein except that it does not comprise one or more particular genetic modifications engineered into the modified host cell, for example, one or more modifications selected from the group consisting of: heterologous expression of an enzyme of a steviol pathway, steviol glycoside pathway, heterologous expression of an enzyme of a steviol glycoside pathway, heterologous expression of a geranylgeranyl diphosphate synthase, heterologous expression of a copalyl diphosphate synthase, heterologous expression of a kaurene synthase, heterologous expression of a kaurene oxidase, heterologous expression of a steviol synthase (kaurenoic acid hydroxylase), heterologous expression of a cytochrome P450 reductase, heterologous expression of a UDP-glycosyltransferase including for example EUGT11, UGT74G1, UGT76G1, UGT85C2, UGT91D, and UGT40087 or variants thereof.

As used herein, the term “medium” refers to culture medium and/or fermentation medium.

As used herein, the term “fermentation composition” refers to a composition which comprises genetically modified host cells and products or metabolites produced by the genetically modified host cells. An example of a fermentation composition is a whole cell broth, which may be the entire contents of a vessel, including cells, aqueous phase, and compounds produced from the genetically modified host cells.

As used herein, the term “production” generally refers to an amount of steviol glycoside produced by a genetically modified host cell provided herein. In some embodiments, production is expressed as a yield of steviol glycoside by the host cell. In other embodiments, production is expressed as the productivity of the host cell in producing the steviol glycoside.

As used herein, the term “yield” refers to production of a steviol glycoside by a host cell, expressed as the amount of steviol glycoside produced per amount of carbon source consumed by the host cell, by weight.

As used herein, the term “productivity” refers to production of steviol glycoside by a host cell, expressed as the amount of steviol glycoside produced (by weight) per amount of fermentation broth in which the host cell is cultured (by volume) over time (per hour).

As used herein, the term “signal sequence” or “N-terminal signal sequence” refers to a short peptide (e.g., 5-50 amino acids in length) at the N-terminus of a polypeptide that directs a polypeptide towards the secretory pathway (e.g., the extracellular space). The signal peptide is typically cleaved during secretion of the polypeptide. The signal sequence may direct the polypeptide to an intracellular compartment or organelle, e.g., the endoplasmic reticulum. A signal sequence may be identified by homology, or biological activity, to a peptide with the known function of targeting a polypeptide to a particular region of the cell. One of ordinary skill in the art can identify a signal peptide by using readily available software (e.g., Sequence Analysis Software Package of the Genetics Computer Group, University of Wisconsin Biotechnology Center, 1710 University Avenue, Madison, Wis. 53705, BLAST, or PILEUP/PRETTYBOX programs). A signal peptide can be one that is, for example, substantially identical to the amino acid sequence of SEQ ID NO: 22 or the amino acids 1-25 of SEQ ID NO: 1. An N-terminal signal sequence may be replaced with a corresponding amino acid sequence encoding a heterologous N-terminal signal sequence (e.g., an N-terminal signal sequence from plant p450 polypeptide).

As used herein, the term “kaurenoic acid” refers to the compound kaurenoic acid, including any stereoisomer of kaurenoic acid. In preferred embodiments, the term refers to the enantiomer known in the art as ent-kaurenoic acid and having the following structure:

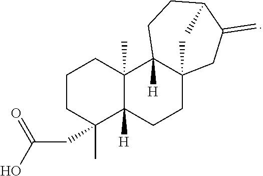

As used herein, the term “steviol” refers to the compound steviol, including any stereoisomer of steviol. In preferred embodiments, the term refers to the compound having the following structure:

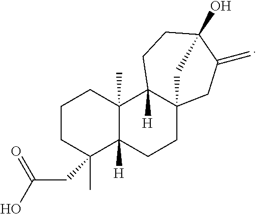

As used herein, the term “steviol glycoside” refers to a glycoside of steviol including but not limited to 19-glycoside, steviolmonoside, steviolbioside, rubusoside, dulcoside B, dulcoside A, rebaudioside A, rebaudioside B, rebaudioside C, rebaudioside D, rebaudioside E, rebaudioside F, rebaudioside G, rebaudioside H, rebaudioside I, rebaudioside J, rebaudioside K, rebaudioside L, rebaudioside M, rebaudioside N, rebaudioside O, rebaudioside D2, and rebaudioside M2.

As used herein, the term “rebaudioside M” or “Reb M” refers to a steviol glycoside having the following structure:

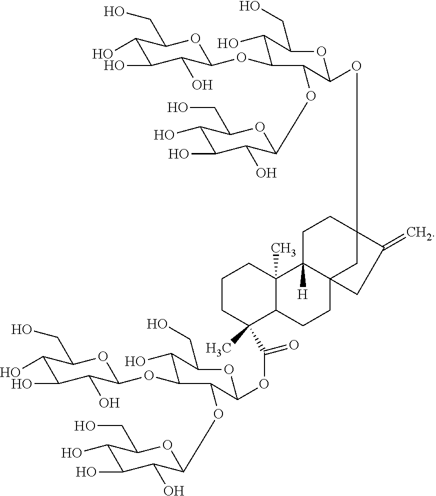

As used herein, the term “sequence identity” or “percent identity” in the context of two or more polynucleotide or polypeptide sequences, refers to two or more sequences or subsequences that are the same or have a specified percentage of nucleotides or amino acid residues that are the same. For example, the sequence may have a percent identity of at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or higher identity over a specified region to a reference sequence when compared and aligned for maximum correspondence over a comparison window, or designated region as measured using a sequence comparison algorithm or by manual alignment and visual inspection. For example, percent identity is determined by calculating the ratio of the number of identical nucleotides (or amino acid residues) in the sequence divided by the length of the total nucleotides (or amino acid residues) minus the lengths of any gaps.

For convenience, the extent of identity between two sequences can be ascertained using computer programs and mathematical algorithms known in the art. Such algorithms that calculate percent sequence identity generally account for sequence gaps and mismatches over the comparison region. Programs that compare and align sequences, like Clustal W (Thompson et al. (1994) Nuclei Acids Res., vol. 22, pp. 4673-4680), ALIGN (Myers et al., (1988) (CABIOS, vol. 4, pp. 11-17), FASTA (Pearson et al., (1988) PNAS, vol. 85, pp. 2444-2448; Pearson (1990) Methods Enzymol., vol. 183, pp. 63-98), and gapped BLAST (Altschul et al., (1997) Nucleic Acids Res., vol. 25, pp. 3389-3402) are useful for this purpose. The BLAST or BLAST 2.0 (Altschul et al., (1990) J. Mol. Biol., vol. 215 pp. 403-410) are available from several sources, including the National Center for Biological Information (NCBI) and on the Internet, for use in connection with the sequence analysis programs BLASTP, BLASTN, BLASTX, TBLASTN, and TBLASTX. Additional information can be found at the NCBI web site.

In certain embodiments, the sequence alignments and percent identity calculations can be determined using the BLAST program using its standard, default parameters. For nucleotide sequence alignment and sequence identity calculations, the BLASTN program is used with its default parameters (Gap opening penalty=5, Gap extension penalty=2, Nucleic match=2, Nucleic mismatch=−3, Expectation value=10.0, Word size=11, Max matches in a query range=0). For polypeptide sequence alignment and sequence identity calculations, BLASTP program is used with its default parameters (Alignment matrix=BLOSUM62; Gap costs: Existence=11, Extension=1; Compositional adjustments=Conditional compositional score, matrix adjustment; Expectation value=10.0; Word size=6; Max matches in a query range=0). Alternatively, the following program and parameters can be used: Align Plus software of Clone Manager Suite, version 5 (Sci-Ed Software); DNA comparison: Global comparison, Standard Linear Scoring matrix, Mismatch penalty=2, Open gap penalty=4, Extend gap penalty=1. Amino acid comparison: Global comparison, BLOSUM 62 Scoring matrix. In the embodiments described herein, the sequence identity is calculated using BLASTN or BLASTP programs using their default parameters. In the embodiments described herein, the sequence alignment of two or more sequences are performed using Clustal W using the suggested default parameters (Dealign input sequences: no; Mbed-like clustering guide-tree: yes; Mbed-like clustering iteration: yes; number of combined iterations: default (0); Max guide tree iterations: default; Max HMM iterations: default; Order: input).

In certain embodiments of the invention, the parental host cell may comprise one or more enzymatic pathways capable of making kaurenoic acid. As described herein, the host cells comprise a Rubus occidentalis kaurenoic acid hydroxylase and variants thereof provided herein, capable of converting kaurenoic acid to steviol. In some embodiments, the host cell further comprises one or more enzymes capable of converting farnesyl diphosphate to geranylgeranyl diphosphate. In further embodiments, the host cell comprises one or more enzymes capable of converting copalyl diphosphate to kaurene. In certain embodiments, the host cell further comprises one or more enzymes capable of converting kaurene to kaurenoic acid. In certain embodiments, the host cell further comprises one or more enzymes capable of converting steviol to one or more steviol glycosides. In certain embodiments, the host cell further comprises one, two, three, four, or more enzymes together capable of converting steviol to Reb A. In certain embodiments, the host cell further comprises one or more enzymes capable of converting Reb A to Reb D. In certain embodiments, the host cell further comprises one or more enzymes capable of converting Reb D to Reb M. Useful enzymes and nucleic acids encoding the enzymes are known to those of skill in the art. Particularly useful enzymes and nucleic acids are described in the sections below and further described, for example in US2014/0329281 A1, US2014/0357588 A1, US2015/0159188, WO2016/038095 A2, and US2016/0198748 A1.

In further embodiments, the host cells further comprise one or more enzymes capable of making geranylgeranyl diphosphate from a carbon source. These include enzymes of the DXP pathway and enzymes of the MEV pathway. Useful enzymes and nucleic acids encoding the enzymes are known to those of skill in the art. Exemplary enzymes of each pathway are described below and further described, for example, in US2016/0177341 A1 which is incorporated by reference herein in its entirety.

In some embodiments, the host cells comprise one or more or all of the isoprenoid pathway enzymes selected from the group consisting of: (a) an enzyme that condenses two molecules of acetyl-coenzyme A to form acetoacetyl-CoA (e.g., an acetyl-coA thiolase); (b) an enzyme that condenses acetoacetyl-CoA with another molecule of acetyl-CoA to form 3-hydroxy-3-methylglutaryl-CoA (HMG-COA) (e.g., an HMG-COA synthase); (c) an enzyme that converts HMG-COA into mevalonate (e.g., an HMG-COA reductase); (d) an enzyme that converts mevalonate into mevalonate 5-phosphate (e.g., a mevalonate kinase); (e) an enzyme that converts mevalonate 5-phosphate into mevalonate 5-pyrophosphate (e.g., a phosphomevalonate kinase); (f) an enzyme that converts mevalonate 5-pyrophosphate into isopentenyl diphosphate (IPP) (e.g., a mevalonate pyrophosphate decarboxylase); (g) an enzyme that converts IPP into dimethylallyl pyrophosphate (DMAPP) (e.g., an IPP isomerase); (h) a polyprenyl synthase that can condense IPP and/or DMAPP molecules to form polyprenyl compounds containing more than five carbons; (i) an enzyme that condenses IPP with DMAPP to form geranyl pyrophosphate (GPP) (e.g., a GPP synthase); (j) an enzyme that condenses two molecules of IPP with one molecule of DMAPP (e.g., an FPP synthase); (k) an enzyme that condenses IPP with GPP to form farnesyl pyrophosphate (FPP) (e.g., an FPP synthase); (1) an enzyme that condenses IPP and DMAPP to form geranylgeranyl pyrophosphate (GGPP); and (m) an enzyme that condenses IPP and FPP to form GGPP.

In certain embodiments, the additional enzymes are native. In advantageous embodiments, the additional enzymes are heterologous. In certain embodiments, two or more enzymes may be combined in one polypeptide.

### Cell Strains

Host cells of the invention provided herein include archae, prokaryotic, and eukaryotic cells.

Suitable prokaryotic host cells include, but are not limited to, any of a gram-positive, gram-negative, and gram-variable bacteria. Examples include, but are not limited to, cells belonging to the genera: Agrobacterium, Alicyclobacillus, Anabaena, Anacystis, Arthrobacter, Azobacter, Bacillus, Brevibacterium, Chromatium, Clostridium, Corynebacterium, Enterobacter, Erwinia, Escherichia, Lactobacillus, Lactococcus, Mesorhizobium, Methylobacterium, Microbacterium, Phormidium, Pseudomonas, Rhodobacter, Rhodopseudomonas, Rhodospirillum, Rhodococcus, Salmonella, Scenedesmun, Serratia, Shigella, Staphylococcus, Streptomyces, Synechococcus, and Zymomonas. Examples of prokaryotic strains include, but are not limited to: Bacillus subtilis, Bacillus amyloliquefacines, Brevibacterium ammoniagenes, Brevibacterium immariophilum, Clostridium beijerinckii, Enterobacter sakazakii, Escherichia coli, Lactococcus lactis, Mesorhizobium loti, Pseudomonas aeruginosa, Pseudomonas mevalonii, Pseudomonas pudica, Rhodobacter capsulatus, Rhodobacter sphaeroides, Rhodospirillum rubrum, Salmonella enterica, Salmonella typhi, Salmonella typhimurium, Shigella dysenteriae, Shigella flexneri, Shigella sonnei, and Staphylococcus aureus. In a particular embodiment, the host cell is an Escherichia coli cell.

Suitable archae hosts include, but are not limited to, cells belonging to the genera: Aeropyrum, Archaeoglobus, Halobacterium, Methanococcus, Methanobacterium, Pyrococcus, Sulfolobus, and Thermoplasma. Examples of archae strains include, but are not limited to: Archaeoglobus fulgidus, Halobacterium sp., Methanococcus jannaschii, Methanobacterium thermoautotrophicum, Thermoplasma acidophilum, Thermoplasma volcanium, Pyrococcus horikoshii, Pyrococcus abyssi, and Aeropyrum pernix.

Suitable eukaryotic hosts include, but are not limited to, fungal cells, algal cells, insect cells, and plant cells. In some embodiments, yeasts useful in the present methods include yeasts that have been deposited with microorganism depositories (e.g. IFO, ATCC, etc.) and belong to the genera Aciculoconidium, Ambrosiozyma, Arthroascus, Arxiozyma, Ashbya, Babjevia, Bensingtonia, Botryoascus, Botryozyma, Brettanomyces, Bullera, Bulleromyces, Candida, Citeromyces, Clavispora, Cryptococcus, Cystofilobasidium, Debaryomyces, Dekkara, Dipodascopsis, Dipodascus, Eeniella, Endomycopsella, Eremascus, Eremothecium, Erythrobasidium, Fellomyces, Filobasidium, Galactomyces, Geotrichum, Guilliermondella, Hanseniaspora, Hansenula, Hasegawaea, Holtermannia, Hormoascus, Hyphopichia, Issatchenkia, Kloeckera, Kloeckeraspora, Kluyveromyces, Kondoa, Kuraishia, Kurtzmanomyces, Leucosporidium, Lipomyces, Lodderomyces, Malasserzia, Metschnikowia, Mrakia, Myxozyma, Nadsonia, Nakazawaea, Nematospora, Ogataea, Oosporidium, Pachysolen, Phachytichospora, Phaffia, Pichia, Rhodosporidium, Rhodotorula, Saccharomyces, Saccharomycodes, Saccharomycopsis, Saitoella, Sakaguchia, Saturnospora, Schizoblastoporion, Schizosaccharomyces, Schwanniomyces, Sporidiobolus, Sporobolomyces, Sporopachydermia, Stephanoascus, Sterigmatomyces, Sterigmatosporidium, Symbiotaphrina, Sympodiomyces, Sympodiomycopsis, Torulaspora, Trichosporiella, Trichosporon, Trigonopsis, Tsuchiyaea, Udeniomyces, Waltomyces, Wickerhamia, Wickerhamiella, Williopsis, Yamadazyma, Yarrowia, Zygoascus, Zygosaccharomyces, Zygowilliopsis, and Zygozyma.

In some embodiments, the host microbe is Saccharomyces cerevisiae, Pichia pastoris, Schizosaccharomyces pombe, Dekkera bruxellensis, Kluyveromyces lactis (previously called Saccharomyces lactis), Kluveromyces marxianus, Arxula adeninivorans, or Hansenula polymorpha (now known as Pichia angusta). In some embodiments, the host microbe is a strain of the genus Candida, such as Candida lipolytica, Candida guilliermondii, (Candida krusei, Candida pseudotropicalis, or Candida utils.

In preferred embodiments, the host microbe is Saccharomyces cerevisiae. In some embodiments, the host is a strain of Saccharomyces cerevisiae selected from Baker's yeast, CEN.PK2, CBS 7959, CBS 7960, CBS 7961, CBS 7962, CBS 7963, CBS 7964, IZ-1904, TA, BG-1, CR-1, SA-1, M-26, Y-904, PE-2, PE-5, VR-1 BR-1, BR-2, ME-2, VR-2, MA-3, MA-4, CAT-1, CB-1, NR-1, BT-1, and AL-1. In some embodiments, the host microbe is a strain of Saccharomyces cerevisiae selected from PE-2, CAT-1, VR-1, BG-1, CR-1, and SA-1. In a particular embodiment, the strain of Saccharomyces cerevisiae is PE-2. In another particular embodiment, the strain of Saccharomyces cerevisiae is CAT-1. In another particular embodiment, the strain of Saccharomyces cerevisiae is BG-1.

### The Steviol Glycoside Biosynthesis Pathway

In some embodiments, a steviol glycoside biosynthesis pathway is activated in the genetically modified host cells by engineering the cells to express polynucleotides encoding enzymes capable of catalyzing the biosynthesis of steviol glycosides.

In some embodiments, the genetically modified host cells contain a heterologous polynucleotide encoding geranylgeranyl diphosphate synthase (GGPPS), a heterologous polynucleotide encoding copalyl diphosphate synthase (CDPS), a heterologous polynucleotide encoding kaurene synthase (KS), a heterologous polynucleotide encoding kaurene oxidase (KO), a heterologous polynucleotide encoding kaurene acid hydroxylase (KAH), a heterologous polynucleotide encoding cytochrome P450 reductase (CPR), a heterologous polynucleotide encoding a UDP-glucose transferase, a heterologous polynucleotide encoding UGT74G1, a heterologous polynucleotide encoding UGT76G1, a heterologous polynucleotide encoding UGT85C2, a heterologous polynucleotide encoding UGT91D, a heterologous polynucleotide encoding EUGT11, and/or a heterologous polynucleotide encoding UGT40087. In some embodiments, the genetically modified host cells contain a heterologous polynucleotide encoding a variant GGPPS, CDPS, KS, KO, KAH, CPR, UDP-glucose transferase, UGT74G1, UGT76G1, UGT85C2, UGT91D, EUGT11, and/or UGT40087. In certain embodiments, the variant enzyme may have from 1 up to 20 amino acid substitutions relative to a reference enzyme. In certain embodiments, the coding sequence of the polynucleotide is codon optimized for the particular host cell.

### Geranylgeranyl Diphosphate Synthase (GGPPS)

Geranylgeranyl diphosphate synthases (EC 2.5.1.29) catalyze the conversion of farnesyl pyrophosphate into geranylgeranyl diphosphate. Examples of geranylgeranyl diphosphate synthase include those of Stevia rebaudiana (accession no. ABD92926), Gibberella fujikuroi (accession no. CAA75568), Mus musculus (accession no. AAH69913), Thalassiosira pseudonana (accession no. XP_002288339), Streptomyces clavuligerus (accession no. ZP-05004570), Sulfolobus acidocaldarius (accession no. BAA43200), Synechococcus sp. (accession no. ABC98596), Arabidopsis thaliana (accession no. MP 195399), and Blakeslea trispora (accession no. AFC92798.1), and those described in US2014/0329281 A1.

### Copalyl Diphosphate Synthase (CDPS)

Copalyl diphosphate synthases (EC 5.5.1.13) catalyze the conversion of geranylgeranyl diphosphate into copalyl diphosphate. Examples of copalyl diphosphate synthases include those from Stevia rebaudiana (accession no. AAB87091), Streptomyces clavuligerus (accession no. EDY51667), Bradyrhizobium japonicum (accession no. AAC28895.1), Zea mays (accession no. AY562490), Arabidopsis thaliana (accession no. NM_116512), and Oryza sativa (accession no. Q5MQ85.1), and those described in US2014/0329281 A1.

### Kaurene Synthase (KS)

Kaurene synthases (EC 4.2.3.19) catalyze the conversion of copalyl diphosphate into kaurene and diphosphate. Examples of enzymes include those of Bradyrhizobium japonicum (accession no. AAC28895.1), Arabidopsis thaliana (accession no. Q9SAK2), and Picea glauca (accession no. ADB55711.1), and those described in US2014/0329281 A1.

Bifunctional copalyl diphosphate synthase (CDPS) and kaurene synthase (KS)

CDPS-KS bifunctional enzymes (EC 5.5.1.13 and EC 4.2.3.19) may also be used in the host cells of the invention. Examples include those of Phomopsis amygdali (accession no. BAG30962), Phaeosphaeria sp. (accession no. 013284), Physcomitrella patens (accession no. BAF61135), and Gibberella fujikuroi (accession no. Q9UVY5.1), and those described in US2014/032928 A1, US2014/0357588 A1, US2015/0159188, and WO2016/038095.

### Ent-Kaurene Oxidase (KO)

Ent-kaurene oxidases (EC 1.14.13.88) also referred to as kaurene oxidases herein catalyze the conversion of kaurene into kaurenoic acid. Illustrative examples of enzymes include those of Oryza sativa (accession no. Q5Z5R4), Gibberella fujikuroi (accession no. 094142), Arabidopsis thaliana (accession no. Q93ZB2), Stevia rebaudiana (accession no. AAQ63464.1), and Pisum sativum (Uniprot no. Q6XAF4), and those described in US2014/0329281 A1, US2014/0357588 A1, US2015/0159188, and WO2016/038095.

### Kaurenoic Acid Hydroxylase (KAH)

Kaurenoic acid hydroxylases (EC 1.14.13) also referred to as steviol synthases catalyze the conversion of kaurenoic acid into steviol. Examples of enzymes include those of Stevia rebaudiana (accession no. ACD93722), Arabidopsis thaliana (accession no. NP_197872), Vitis vinifera (accession no. XP_002282091), and Medicago trunculata (accession no. ABC59076), and those described in US2014/0329281, US2014/0357588, US2015/0159188, and WO2016/038095. In some embodiments, the kaurenoic acid hydroxylase polypeptide may be a variant KAH. In some embodiments, the variant KAH comprises the amino acid sequence of SEQ ID NO: 1. In some embodiments, the amino acid sequence of SEQ ID NO: 1 further includes one or more amino acid substitutions. In some embodiments, the one or more amino acid substitutions are selected from K69R, V343G, T403V, H491P, P72D, L64D, Q84C, L64G, E206D, Y238C, A210G, L64N, I237C, L11V, N207F, M73G, W8G, E60R, Y55S, N475G, D292P, P161C, K267D, L485F, A396F, R507A, P72T, I132G, N61P, K119V, T220E, P72G, Q513R, S133G, Y506V, K69P, E60G, K224C, M73H, H379G, P72C, K314P, W202A, G466F, N49A, S339G, N160D, T216G, D102Y, F246G, M58P, T220R, R458D, M58G, A68I, S70P, F88V, T240D, L205I, K167G, L232M, S62R, G56D, Q244G, A242D, N49R, Q513G, W29T, L303D, T378D, I508L, W202Q, S505R, R233C, I104D, M258G, K69G, F88D, F88S, A217V, E230C, R507G, G466S, G56S, E230G, Y55G, A503C, S460I, I129R, S245G, F246S, Q84L, S133R, T509V, R507E, R233T, V30F, A68G, G56N, T162G, A68P, S165D, K119Y, W29C, S165P, W29V, I284G, A217L, Q335V, L65S, F53R, Y55P, W202V, K224V, W29A, H164G, Q244D, K291C, L65G, K167S, C327I, K291S, D57G, K167H, N160T, W202C, A242G, F88R, I104N, G466D, N475D, K119S, T123D, T216A, S339A, P161D, I104R, L54G, M171F, L232Y, D293C, V340A, T162A, A297V, I104H, F332L, A236R, K224I, S452D, I104A, V340S, F229Y, A297Y, and A297F. In some embodiments, the one or more amino acid substitutions are selected from A297V, I104H, F332L, A236R, K224I, S452D, I104A, I104A, V340S, F229Y, A297Y, and A297F. In some embodiments, the one or more amino acid substitutions are selected from S452D, I104A, V340S, F229Y, A297Y, and A297F. In some embodiments, the one or more amino acid substitutions are selected from N146W, A297Y, A236S, V9S, G466F, T283D, T142G, T425V, L459C, T283A, T283G, S460V, S133G, I129V, W52G, S505I, 1243T, V340S, S460C, S452D, L118I, S505V, T123D, W52C, S460I, S457G, W52R, W52N, N146T, G466A, and W52T. In some embodiments, the one or more amino acid substitutions are selected from N146T/A297Y/G466A; W52T/N146T/A297Y; T142G/N146T/A297Y/G466A; W52T/T142G/N146T/A297Y; and W52T/T142G/N146T/A297Y/G466A. In some embodiments, the amino acid substitutions include N146T/A297Y/G466A. In some embodiments, the amino acid substitutions include W52T/N146T/A297Y. In some embodiments, the amino acid substitutions include T142G/N146T/A297Y/G466A. In some embodiments, the amino acid substitutions include W52T/T142G/N146T/A297Y. In some embodiments, the amino acid substitutions include W52T/T142G/N146T/A297Y/G466A. In some embodiments, the amino acid substitutions include W52T/T142G/G466A. In some embodiments, the amino acid substitutions include A145G/N146F/A297Y. In some embodiments, the amino acid substitutions include N146W/A297Y/S460I. In some embodiments, the amino acid substitutions include W52N/N146W/A297Y. In some embodiments, the amino acid substitutions include F332L/S452D. In some embodiments, the amino acid substitutions include N146W/A297Y. In some embodiments, the amino acid substitutions comprise A297Y. In some embodiments, the amino acid substitutions include Q84R/N146T/A297Y. In some embodiments, the variant polypeptide includes the amino acid sequence selected from SEQ ID NO: 17, SEQ ID NO: 18, SEQ ID NO: 19, SEQ ID NO: 20, and SEQ ID NO: 21. In some embodiments, the variant polypeptide includes the amino acid sequence of SEQ ID NO: 17. In some embodiments, the variant polypeptide includes the amino acid sequence of SEQ ID NO: 18. In some embodiments, the variant polypeptide includes the amino acid sequence of SEQ ID NO: 19. In some embodiments, the variant polypeptide includes the amino acid sequence of SEQ ID NO: 20. In some embodiments, the variant polypeptide includes the amino acid sequence of SEQ ID NO: 21. In some embodiments, the amino acids encoding an N-terminal signal sequence are replaced with a corresponding amino acid sequence encoding the N-terminal signal sequence of a heterologous plant p450 polypeptide. In some embodiments, the N-terminal signal sequence corresponds to amino acids 1-25 of SEQ ID NO: 1. In some embodiments, the amino acid sequence corresponding to the N-terminal signal sequence includes SEQ ID NO: 22.

### Cytochrome P450 Reductase (CPR)

Cytochrome P450 reductases (EC 1.6.2.4) are necessary for the activity of KO and/or KAH above. Examples of enzymes include those of Stevia rebaudiana (accession no. ABB88839), Arabidopsis thaliana (accession no. NP_194183), Gibberella fujikuroi (accession no. CAE09055), and Artemisia annua (accession no. ABC47946.1), and those described in US2014/0329281, US2014/0357588, US2015/0159188, and WO2016/038095.

### UDP Glycosyltransferase 74G1 (UGT74G1)

UGT74G1 is capable of functioning as a uridine 5′-diphospho glucosyl: steviol 19-COOH transferase and as a uridine 5′-diphospho glucosyl: steviol-13-O-glucoside 19-COOH transferase. Accordingly, UGT74G1 is capable of converting steviol to 19-glycoside; converting steviol to 19-glycoside, steviolmonoside to rubusoside; and steviolbioside to stevioside. UGT74G1 has been described in Richman et al., 2005, Plant J., vol. 41, pp. 56-67; US2014/0329281; WO2016/038095; and accession no. AAR06920.1.

### UDP Glycosyltransferase 76G1 (UGT76G1)

UGT76G1 is capable of transferring a glucose moiety to the C-3′ position of an acceptor molecule a steviol glycoside (where glycoside=Glcb(1->2) Glc). This chemistry can occur at either the C-13-O-linked glucose of the acceptor molecule, or the C-19-O-linked glucose acceptor molecule. Accordingly, UGT76G1 is capable of functioning as a uridine 5′-diphospho glucosyltransferase to the: (1) C-3′ position of the 13-O-linked glucose on steviolbioside in a beta linkage forming Reb B, (2) C-3′ position of the 19-O-linked glucose on stevioside in a beta linkage forming Reb A, and (3) C-3′ position of the 19-O-linked glucose on Reb D in a beta linkage forming Reb M. UGT76G1 has been described in Richman et al., 2005, Plant J., vol. 41, pp. 56-67; US2014/0329281; WO2016/038095; and accession no. AAR06912.1.

### UDP Glycosyltransferase 85C2 (UGT85C2)

UGT85C2 is capable of functioning as a uridine 5′-diphospho glucosyl: steviol 13-OH transferase, and a uridine 5′-diphospho glucosyl: steviol-19-O-glucoside 13-OH transferase. UGT85C2 is capable of converting steviol to steviolmonoside and is also capable of converting 19-glycoside to rubusoside. Examples of UGT85C2 enzymes include those of Stevia rebaudiana: see e.g., Richman et al., (2005), Plant J., vol. 41, pp. 56-67; US2014/0329281; WO2016/038095; and accession no. AAR06916.1.

### UDP Glycosyltransferase 91D (UGT91D)

UGT91D is capable of functioning as a uridine 5′-diphosphoglucosyl: steviol-13-O-glucoside transferase, transferring a glucose moiety to the C-2′ of the 13-O-glucose of the acceptor molecule, steviol-13-O-glucoside (steviolmonoside) to produce steviolbioside. A UGT91D is also capable of functioning as a uridine 5′-diphosphoglucosyl: rubusoside transferase, transferring a glucose moiety to the C-2′ of the 13-O-glucose of the acceptor molecule, rubusoside, to provide stevioside. UGT91D is also referred to as UGT91D2, UGT91D2e, or UGT91D-like3. Examples of UGT91D enzymes include those of Stevia rebaudiana: see e.g., accession no. ACE87855.1; US2014/0329281; and WO2016/038095.

### UDP Glycosyltransferase 40087 (UGT40087)

UGT40087 is capable of transferring a glucose moiety to the C-2′ position of the 19-O-glucose of Reb A to produce Reb D. UGT40087 is also capable of transferring a glucose moiety to the C-2′ position of the 19-O-glucose of stevioside to produce Reb E. Examples of UGT40087 include those of accession no. XP_004982059.1 and WO2018/031955.

### Additional Uridine Diphosphate-Dependent Glycosyl Transferases Capable of Converting Reb A to Reb D (UGTAD)

In addition to UGT40087, other UGTAD are capable of transferring a glucose moiety to the C-2′ position of 19-O-glucose of Reb A to produce Reb D. UGTAD is also capable of transferring a glucose moiety to the C-2′ position of 19-O-glucose of stevioside to produce Reb E. Examples of UGTAD include Os_UGT_91C1 from Oryza sativa (also referred to as EUGT11 (see WO2013/022989 and accession number XP_01529141.1)); S1 UGT_101249881 from Solanum lycopersicum (also referred to as UGTSL2 (see WO2014/193888 and accession no. XP_0042504851)); sr.UGT_925778; Bd_UGT0840 (see accession no. XP_003560669.1); Hv_UGT_VI (see accession no. BAJ94055.1); Bd_UGT10850 (see accession no. XP_010230871.1); and OB_UGT91B1_like (see accession no. XP_0066504551.).

MEV Pathway FPP and/or GGPP Production

In some embodiments, a genetically modified host cell provided herein comprises one or more heterologous enzymes of the MEV pathway, useful for the formation of FPP and/or GGPP. The one or more enzymes of the MEV pathway may include an enzyme that condenses acetyl-CoA with malonyl-CoA to form acetoacetyl-CoA; an enzyme that condenses two molecules of acetyl-CoA to form acetoacetyl-CoA; an enzyme that condenses acetoacetyl-CoA with acetyl-CoA to form HMG-COA; or an enzyme that converts HMG-CoA to mevalonate. In addition, the genetically modified host cells may include a MEV pathway enzyme that phosphorylates mevalonate to mevalonate 5-phosphate; a MEV pathway enzyme that converts mevalonate 5-phosphate to mevalonate 5-pyrophosphate; a MEV pathway enzyme that converts mevalonate 5-pyrophosphate to isopentenyl pyrophosphate; or a MEV pathway enzyme that converts isopentenyl pyrophosphate to dimethylallyl diphosphate. In particular, the one or more enzymes of the MEV pathway are selected from acetyl-CoA thiolase, acetoacetyl-CoA synthetase, HMG-COA synthase, HMG-CoA reductase, mevalonate kinase, phosphomevalonate kinase, mevalonate pyrophosphate decarboxylase, and isopentyl diphosphate: dimethylallyl diphosphate isomerase (IDI or IPP isomerase). The genetically modified host cell of the invention may express one or more of the heterologous enzymes of the MEV from one or more heterologous nucleotide sequences comprising the coding sequence of the one or more MEV pathway enzymes.

In some embodiments, the genetically modified host cell comprises a heterologous nucleic acid encoding an enzyme that can convert isopentenyl pyrophosphate (IPP) into dimethylallyl pyrophosphate (DMAPP). In addition, the host cell may contain a heterologous nucleic acid encoding an enzyme that may condense IPP and/or DMAPP molecules to form a polyprenyl compound. In some embodiments, the genetically modified host cell further contains a heterologous nucleic acid encoding an enzyme that may modify IPP or a polyprenyl to form an isoprenoid compound such as FPP.

### Conversion of Acetyl-CoA to Acetoacetyl-CoA

The genetically modified host cell may contain a heterologous nucleic acid that encodes an enzyme that may condense two molecules of acetyl-coenzyme A to form acetoacetyl-CoA (an acetyl-CoA thiolase). Examples of nucleotide sequences encoding acetyl-CoA thiolase include (accession no. NC_000913 REGION: 2324131.2325315 (Escherichia coli)); (D49362 (Paracoccus denitrificans)); and (L20428 (Saccharomyces cerevisiae)).

Acetyl-CoA thiolase catalyzes the reversible condensation of two molecules of acetyl-CoA to yield acetoacetyl-CoA, but this reaction is thermodynamically unfavorable; acetoacetyl-CoA thiolysis is favored over acetoacetyl-CoA synthesis. Acetoacetyl-CoA synthase (AACS) (also referred to as acetyl-CoA: malonyl-CoA acyltransferase; EC 2.3.1.194) condenses acetyl-CoA with malonyl-CoA to form acetoacetyl-CoA. In contrast to acetyl-CoA thiolase, AACS-catalyzed acetoacetyl-CoA synthesis is essentially an energy-favored reaction, due to the associated decarboxylation of malonyl-CoA. In addition, AACS exhibits no thiolysis activity against acetoacetyl-CoA, and thus the reaction is irreversible.

In cells expressing acetyl-CoA thiolase and a heterologous ADA and/or phosphotransacetylase (PTA), the reversible reaction catalyzed by acetyl-CoA thiolase, which favors acetoacetyl-CoA thiolysis, may result in a large acetyl-CoA pool. In view of the reversible activity of ADA, this acetyl-CoA pool may in turn drive ADA towards the reverse reaction of converting acetyl-CoA to acetaldehyde, thereby diminishing the benefits provided by ADA towards acetyl-CoA production. Similarly, the activity of PTA is reversible, and thus, a large acetyl-CoA pool may drive PTA towards the reverse reaction of converting acetyl-CoA to acetyl phosphate. Therefore, in some embodiments, in order to provide a strong pull on acetyl-CoA to drive the forward reaction of ADA and PTA, the MEV pathway of the genetically modified host cell provided herein utilizes an acetoacetyl-CoA synthase to form acetoacetyl-CoA from acetyl-CoA and malonyl-CoA.

The AACS obtained from Streptomyces sp. Strain CL 190 may be used (see Okamura et al., (2010), PNAS, vol. 107, pp. 11265-11270). Representative AACS encoding nucleic acids sequences from Streptomyces sp. Strain CL 190 include the sequence of accession no. AB540131.1, and the corresponding AACS protein sequences include the sequence of accession nos. D7URV0 and BAJ10048. Other acetoacetyl-CoA synthases useful for the invention include those of Streptomyces sp. (see accession nos. AB183750; KO-3988 BAD86806; KO-3988 AB212624; and KO-2988 BAE78983); S. anulatus strain 9663 (see accession nos. FN178498 and CAX48662); Actinoplanes sp. A40644 (see accession nos. AB113568 and BAD07381); Streptomyces sp. C (see accession nos. NZ_ACEW010000640 and ZP_05511702); Nocardiopsis dassonvillei DSM 43111 (see accession nos. NZ ABUI01000023 and ZP_04335288); Mycobacterium ulcerans Agy99 (see accession nos. NC 008611 and YP_907152); Mycobacterium marinum M (see accession nos. NC_010612 and YP_001851502); Streptomyces sp. Mg1 (see accession nos. NZ_DS570501 and ZP_05002626); Streptomyces sp. AA4 (see accession nos. NZ_ACEV01000037 and ZP_05478992); S. roseosporus NRRL 15998 (see accession nos. NZ_ABYB01000295 and ZP_04696763); Streptomyces sp. ACTE (see accession nos. NZ_ADFD01000030 and ZP 06275834); S. viridochromogenes DSM 40736 (see accession nos. NZ_ACEZ01000031 and ZP_05529691); Frankia sp. Ccl3 (see accession nos. NC_007777 and YP_480101); Nocardia brasiliensis (see accession nos. NC_018681 and YP_006812440.1); and Austwickia chelonae (see accession nos. NZ_BAGZ01000005 and ZP_10950493.1). Additional suitable acetoacetyl-CoA synthases include those described in U.S. Patent Application Publication Nos. 2010/0285549 and 2011/0281315.

Acetoacetyl-CoA synthases also useful in the compositions and methods provided herein include those molecules which are said to be “derivatives” of any of the acetoacetyl-CoA synthases described herein. Such a “derivative” has the following characteristics: (1) it shares substantial homology with any of the acetoacetyl-CoA synthases described herein; and (2) is capable of catalyzing the irreversible condensation of acetyl-CoA with malonyl-CoA to form acetoacetyl-CoA. A derivative of an acetoacetyl-CoA synthase is said to share “substantial homology” with acetoacetyl-CoA synthase if the amino acid sequences of the derivative is at least 80%, and more preferably at least 90%, and most preferably at least 95%, the same as that of acetoacetyl-CoA synthase.

### Conversion of Acetoacetyl-CoA to HMG-COA

In some embodiments, the host cell comprises a heterologous nucleotide sequence encoding an enzyme that can condense acetoacetyl-CoA with another molecule of acetyl-CoA to form 3-hydroxy-3-methylglutaryl-CoA (HMG-COA), e.g., an HMG-COA synthase. Examples of nucleotide sequences encoding such an enzyme include: (NC_001145. complement 19061.20536; Saccharomyces cerevisiae), (X96617; Saccharomyces cerevisiae), (X83882; Arabidopsis thaliana), (AB037907; Kitasatospora griseola), (BT007302; Homo sapiens), and (NC_002758, Locus tag SAV2546, GeneID 1122571; Staphylococcus aureus).

### Conversion of HMG-COA to Mevalonate

In some embodiments, the host cell comprises a heterologous nucleotide sequence encoding an enzyme that can convert HMG-COA into mevalonate, e.g., an HMG-COA reductase. The HMG-COA reductase may be an NADH-using hydroxymethylglutaryl-CoA reductase-CoA reductase. HMG-COA reductases (EC 1.1.1.34; EC 1.1.1.88) catalyze the reductive deacylation of(S)-HMG-COA to (R)-mevalonate, and can be categorized into two classes, class I and class II HMGrs. Class I includes the enzymes from eukaryotes and most archaea, and class II includes the HMG-COA reductases of certain prokaryotes and archaea. In addition to the divergence in the sequences, the enzymes of the two classes also differ with regard to their cofactor specificity. Unlike the class I enzymes, which utilize NADPH exclusively, the class II HMG-COA reductases vary in the ability to discriminate between NADPH and NADH (See, e.g., Hedl et al., (2004) Journal of Bacteriology, vol. 186, pp. 1927-1932). Co-factor specificities for select class II HMG-CoA reductases are provided in Table 1.

TABLE 1

| | Coenzyme | | |
|---|---|---|---|
| Source | specificity | Kₘ^{NADPH} (μM) | Kₘ^{NADH} (μM) |
| P. mevalonii | NADH | | 80 |
| A. fulgidus | NAD(P)H | 500 | 160 |
| S. aureus | NAD(P)H | 70 | 100 |
| E. faecalis | NADPH | 30 | |

HMG-COA reductases useful for the invention include HMG-COA reductases that are capable of utilizing NADH as a cofactor, e.g., HMG-COA reductase from P. mevalonii, A. fulgidus, or S. aureus. In particular embodiments, the HMG-COA reductase is capable of only utilizing NADH as a cofactor, e.g., HMG-COA reductase from P. mevalonii, S. pomeroyi, or D. acidovorans.

In some embodiments, the NADH-using HMG-COA reductase is from Pseudomonas mevalonii. The sequence of the wild-type mvaA gene of Pseudomonas mevalonii, which encodes HMG-COA reductase (EC 1.1.1.88), has been previously described (see Beach and Rodwell, (1989), J. Bacteriol., vol. 171, pp. 2994-3001). Representative mvaA nucleotide sequences of Pseudomonas mevalonii include accession number M24015. Representative HMG-COA reductase protein sequences of Pseudomonas mevalonii include accession numbers AAA25837, P13702, and MVAA PSEMV.

In some embodiments, the NADH-using HMG-COA reductase is from Silicibacter pomeroyi. Representative HMG-COA reductase nucleotide sequences of Silicibacter pomeroyi include accession number NC_006569.1. Representative HMG-COA reductase protein sequences of Silicibacter pomeroyi include accession number YP 164994.

In some embodiments, the NADH-using HMG-COA reductase is from Delftia acidovorans. A representative HMG-COA reductase nucleotide sequences of Delftia acidovorans includes NC_010002 REGION: complement (319980 . . . 321269). Representative HMG-COA reductase protein sequences of Delftia acidovorans include accession number YP 001561318.

In some embodiments, the NADH-using HMG-COA reductase is from Solanum tuberosum (see Crane et al., (2002), J. Plant Physiol., vol. 159, pp. 1301-1307).

NADH-using HMG-COA reductases useful in the practice of the invention also include those molecules which are said to be “derivatives” of any of the NADH-using HMG-CoA reductases described herein, e.g., from P. mevalonii, S. pomeroyi and D. acidovorans. Such a “derivative” has the following characteristics: (1) it shares substantial homology with any of the NADH-using HMG-COA reductases described herein; and (2) is capable of catalyzing the reductive deacylation of(S)-HMG-COA to (R)-mevalonate while preferentially using NADH as a cofactor. A derivative of an NADH-using HMG-COA reductase is said to share “substantial homology” with NADH-using HMG-COA reductase if the amino acid sequences of the derivative is at least 80%, and more preferably at least 90%, and most preferably at least 95%, the same as that of NADH-using HMG-COA reductase.

As used herein, the phrase “NADH-using” means that the NADH-using HMG-COA reductase is selective for NADH over NADPH as a cofactor, for example, by demonstrating a higher specific activity for NADH than for NADPH. The selectivity for NADH as a cofactor is expressed as a k_{cat}^{(NADH)}/k_{cat}^{(NADPH)} ratio. The NADH-using HMG-COA reductase of the invention may have a k_{cat}^{(NADH)}/K_{cat}^{(NADPH)} ratio of at least 5, 10, 15, 20, 25 or greater than 25. The NADH-using HMG-COA reductase may use NADH exclusively. For example, an NADH-using HMG-COA reductase that uses NADH exclusively displays some activity with NADH supplied as the sole cofactor in vitro, and displays no detectable activity when NADPH is supplied as the sole cofactor. Any method for determining cofactor specificity known in the art can be utilized to identify HMG-COA reductases having a preference for NADH as cofactor (see e.g., (Kim et al., (2000), Protein Science, vol. 9, pp. 1226-1234) and (Wilding et al., (2000), J. Bacteriol., vol. 182, pp. 5147-5152).

In some cases, the NADH-using HMG-COA reductase is engineered to be selective for NADH over NAPDH, for example, through site-directed mutagenesis of the cofactor-binding pocket. Methods for engineering NADH-selectivity are described in Watanabe et al., (2007), Microbiology, vol. 153, pp. 3044-3054), and methods for determining the cofactor specificity of HMG-COA reductases are described in Kim et al., (2000), Protein Sci., vol. 9, pp. 1226-1234).\

The NADH-using HMG-COA reductase may be derived from a host species that natively comprises a mevalonate degradative pathway, for example, a host species that catabolizes mevalonate as its sole carbon source. In these cases, the NADH-using HMG-CoA reductase, which normally catalyzes the oxidative acylation of internalized (R)-mevalonate to(S)-HMG-COA within its native host cell, is utilized to catalyze the reverse reaction, that is, the reductive deacylation of(S)-HMG-COA to (R)-mevalonate, in a genetically modified host cell comprising a mevalonate biosynthetic pathway. Prokaryotes capable of growth on mevalonate as their sole carbon source have been described by: (Anderson et al., (1989), J. Bacteriol, vol. 171, pp. 6468-6472); (Beach et al., (1989), J. Bacteriol., vol. 171, pp. 2994-3001); Bensch et al., J. Biol. Chem., vol. 245, pp. 3755-3762); (Fimongnari et al., (1965), Biochemistry, vol. 4, pp. 2086-2090); Siddiqi et al., (1962), Biochem. Biophys. Res. Commun., vol. 8, pp. 110-113); (Siddiqi et al., (1967), J. Bacteriol., vol. 93, pp. 207-214); and (Takatsuji et al., (1983), Biochem. Biophys. Res. Commun., vol. 110, pp. 187-193).

The host cell may contain both a NADH-using HMGr and an NADPH-using HMG-CoA reductase. Examples of nucleotide sequences encoding an NADPH-using HMG-COA reductase include: (NM_206548; Drosophila melanogaster), (NC_002758, Locus tag SAV2545, GeneID 1122570; Staphylococcus aureus), (AB015627; Streptomyces sp. KO 3988), (AX128213, providing the sequence encoding a truncated HMG-COA reductase; Saccharomyces cerevisiae), and (NC_001145: complement (115734.118898; Saccharomyces cerevisiae).

### Conversion of Mevalonate to Mevalonate-5-Phosphate

The host cell may contain a heterologous nucleotide sequence encoding an enzyme that can convert mevalonate into mevalonate 5-phosphate, e.g., a mevalonate kinase. Illustrative examples of nucleotide sequences encoding such an enzyme include: (L77688; Arabidopsis thaliana) and (X55875; Saccharomyces cerevisiae).

### Conversion of Mevalonate-5-Phosphate to Mevalonate-5-Pyrophosphate

The host cell may contain a heterologous nucleotide sequence encoding an enzyme that can convert mevalonate 5-phosphate into mevalonate 5-pyrophosphate, e.g., a phosphomevalonate kinase. Illustrative examples of nucleotide sequences encoding such an enzyme include: (AF429385; Hevea brasiliensis), (NM_006556; Homo sapiens), and (NC_001145. complement 712315.713670; Saccharomyces cerevisiae).

### Conversion of Mevalonate-5-Pyrophosphate to IPP

The host cell may contain a heterologous nucleotide sequence encoding an enzyme that can convert mevalonate 5-pyrophosphate into isopentenyl diphosphate (IPP), e.g., a mevalonate pyrophosphate decarboxylase. Illustrative examples of nucleotide sequences encoding such an enzyme include: (X97557; Saccharomyces cerevisiae), (AF290095; Enterococcus faecium), and (U49260; Homo sapiens).

### Conversion of IPP to DMAPP

The host cell may contain a heterologous nucleotide sequence encoding an enzyme that can convert IPP generated via the MEV pathway into dimethylallyl pyrophosphate (DMAPP), e.g., an IPP isomerase. Illustrative examples of nucleotide sequences encoding such an enzyme include: (NC_000913, 3031087.3031635; Escherichia coli), and (AF082326; Haematococcus pluvialis).

### Polyprenyl Synthases

In some embodiments, the host cell further comprises a heterologous nucleotide sequence encoding a polyprenyl synthase that can condense IPP and/or DMAPP molecules to form polyprenyl compounds containing more than five carbons.

The host cell may contain a heterologous nucleotide sequence encoding an enzyme that can condense one molecule of IPP with one molecule of DMAPP to form one molecule of geranyl pyrophosphate (“GPP”), e.g., a GPP synthase. Non-limiting examples of nucleotide sequences encoding such an enzyme include: (AF513111; Abies grandis), (AF513112; Abies grandis), (AF513113; Abies grandis), (AY534686; Antirrhinum majus), (AY534687; Antirrhinum majus), (Y17376; Arabidopsis thaliana), (AE016877, Locus AP11092; Bacillus cereus; ATCC 14579), (AJ243739; Citrus sinensis), (AY534745; Clarkia breweri), (AY953508; Ips pini), (DQ286930; Lycopersicon esculentum), (AF182828; Mentha×piperita), (AF182827; Mentha×piperita), (MPI249453; Mentha×piperita), (PZE431697, Locus CAD24425; Paracoccus zeaxanthinifaciens), (AY866498; Picrorhiza kurrooa), (AY351862; Vitis vinifera), and (AF203881, Locus AAF12843; Zymomonas mobilis).

The host cell may contain a heterologous nucleotide sequence encoding an enzyme that can condense two molecules of IPP with one molecule of DMAPP, or add a molecule of IPP to a molecule of GPP, to form a molecule of farnesyl pyrophosphate (“FPP”), e.g., an FPP synthase. Non-limiting examples of nucleotide sequences that encode an FPP synthase include: (ATU80605; Arabidopsis thaliana), (ATHFPS2R; Arabidopsis thaliana), (AAU36376; Artemisia annua), (AF461050; Bos taurus), (D00694; Escherichia coli K-12), (AE009951, Locus AAL95523; Fusobacterium nucleatum subsp. nucleatum ATCC 25586), (GFFPPSGEN; Gibberella fujikuroi), (CP000009, Locus AAW60034; Gluconobacter oxydans 621H), (AF019892; Helianthus annuus), (HUMFAPS; Homo sapiens), (KLPFPSQCR; Kluyveromyces lactis), (LAU15777; Lupinus albus), (LAU20771; Lupinus albus), (AF309508; Mus musculus), (NCFPPSGEN; Neurospora crassa), (PAFPS1; Parthenium argentatum), (PAFPS2; Parthenium argentatum), (RATFAPS; Rattus norvegicus), (YSCFPP; Saccharomyces cerevisiae), (D89104; Schizosaccharomyces pombe), (CP000003, Locus AAT87386; Streptococcus pyogenes), (CP000017, Locus AAZ51849; Streptococcus pyogenes), (NC_008022, Locus YP_598856; Streptococcus pyogenes MGAS10270), (NC_008023, Locus YP_600845; Streptococcus pyogenes MGAS2096), (NC_008024, Locus YP_602832; Streptococcus pyogenes MGAS10750), (MZEFPS; Zea mays), (AE000657, Locus AAC06913; Aquifex aeolicus VF5), (NM_202836; Arabidopsis thaliana), (D84432, Locus BAA12575; Bacillus subtilis), (U12678, Locus AAC28894; Bradyrhizobium japonicum USDA 110), (BACFDPS; Geobacillus stearothermophilus), (NC_002940, Locus NP_873754; Haemophilus ducreyi 35000HP), (L42023, Locus AAC23087; Haemophilus influenzae Rd KW20), (J05262; Homo sapiens), (YP_395294; Lactobacillus sakei subsp. sakei 23K), (NC_005823, Locus YP_000273; Leptospira interrogans serovar Copenhageni str. Fiocruz L1-130), (AB003187; Micrococcus luteus), (NC_002946, Locus YP_208768; Neisseria gonorrhoeae FA 1090), (U00090, Locus AAB91752; Rhizobium sp. NGR234), (J05091; Saccharomyces cerevisae), (CP000031, Locus AAV93568; Silicibacter pomeroyi DSS-3), (AE008481, Locus AAK99890; Streptococcus pneumoniae R6), and (NC_004556, Locus NP 779706; Xylella fastidiosa Temecula1).

In addition, the host cell may contain a heterologous nucleotide sequence encoding an enzyme that can combine IPP and DMAPP or IPP and FPP to form geranylgeranyl pyrophosphate (“GGPP”). Non-limiting examples of nucleotide sequences that encode such an enzyme include: (ATHGERPYRS; Arabidopsis thaliana), (BT005328; Arabidopsis thaliana), (NM_119845; Arabidopsis thaliana), (NZ_AAJM01000380, Locus ZP_00743052; Bacillus thuringiensis serovar israelensis, ATCC 35646 sq1563), (CRGGPPS; Catharanthus roseus), (NZ_AABF02000074, Locus ZP_00144509; Fusobacterium nucleatum subsp. vincentii, ATCC 49256), (GFGGPPSGN; Gibberella fujikuroi), (AY371321; Ginkgo biloba), (AB055496; Hevea brasiliensis), (AB017971; Homo sapiens), (MCI276129; Mucor circinelloides f. lusitanicus), (AB016044; Mus musculus), (AABX01000298, Locus NCU01427; Neurospora crassa), (NCU20940; Neurospora crassa), (NZ_AAKL01000008, Locus ZP_00943566; Ralstonia solanacearum UW551), (AB118238; Rattus norvegicus), (SCU31632; Saccharomyces cerevisiae), (AB016095; Synechococcus elongates), (SAGGPS; Sinapis alba), (SSOGDS; Sulfolobus acidocaldarius), (NC_007759, Locus YP_461832; Syntrophus aciditrophicus SB), (NC_006840, Locus YP_204095; Vibrio fischeri ES114), (NM_112315; Arabidopsis thaliana), (ERWCRTE; Pantoea agglomerans), (D90087, Locus BAA14124; Pantoea ananatis), (X52291, Locus CAA36538; Rhodobacter capsulatus), (AF195122, Locus AAF24294; Rhodobacter sphaeroides), and (NC_004350, Locus NP_721015; Streptococcus mutans UA159).

While examples of the enzymes of the mevalonate pathway are described above, in certain embodiments, enzymes of the DXP pathway can be used as an alternative or additional pathway to produce DMAPP and IPP in the host cells, compositions and methods described herein. Enzymes and nucleic acids encoding the enzymes of the DXP pathway are well-known and characterized in the art, e.g., WO 2012/135591.

### Methods of Producing Steviol Glycosides

The invention provides for the production of steviol glycosides by (a) culturing a population of any of the genetically modified host cells described herein that are capable of producing a steviol glycoside in a medium with a carbon source under conditions suitable for making the steviol glycoside compound, and (b) recovering the steviol glycoside compound from the medium.

The genetically modified host cell produces an increased amount of the steviol glycoside compared to a parent cell not having the genetic modifications, or a parent cell having only a subset of the genetic modifications but is otherwise genetically identical. In some embodiments, the increased amount is at least 1%, 5%, 10%, 15%, 20%, 25%, 30%, 35%, 40%, 45%, 50%, 55%, 60%, 65%, 70%, 75%, 80%, 85%, 90%, 95%, 100% or greater than 100%, as measured, for example, in yield, production, and/or productivity, in grams per liter of cell culture, milligrams per gram of dry cell weight, on a per unit volume of cell culture basis, on a per unit dry cell weight basis, on a per unit volume of cell culture per unit time basis, or on a per unit dry cell weight per unit time basis.

In some embodiments, the host cell may produce an elevated level of a steviol glycoside that is greater than about 1 gram per liter of fermentation medium. In some embodiments, the host cell produces an elevated level of a steviol glycoside that is greater than about 5 grams per liter of fermentation medium. In some embodiments, the host cell produces an elevated level of a steviol glycoside that is greater than about 10 grams per liter of fermentation medium. In some embodiments, the steviol glycoside is produced in an amount from about 10 to about 50 grams, from about 10 to about 15 grams, more than about 15 grams, more than about 20 grams, more than about 25 grams, or more than about 40 grams per liter of cell culture.

In some embodiments, the host cell produces an elevated level of a steviol glycoside that is greater than about 50 milligrams per gram of dry cell weight. In some such embodiments, the steviol glycoside is produced in an amount from about 50 to about 1500 milligrams, more than about 100 milligrams, more than about 150 milligrams, more than about 200 milligrams, more than about 250 milligrams, more than about 500 milligrams, more than about 750 milligrams, or more than about 1000 milligrams per gram of dry cell weight.

In some embodiments, the host cell produces an elevated level of a steviol glycoside that is at least about 10%, at least about 15%, at least about 20%, at least about 25%, at least about 30%, at least about 35%, at least about 40%, at least about 45%, at least about 50%, at least about 60%, at least about 70%, at least about 80%, at least about 90%, at least about 2-fold, at least about 2.5-fold, at least about 5-fold, at least about 10-fold, at least about 20-fold, at least about 30-fold, at least about 40-fold, at least about 50-fold, at least about 75-fold, at least about 100-fold, at least about 200-fold, at least about 300-fold, at least about 400-fold, at least about 500-fold, or at least about 1,000-fold, or more, higher than the level of steviol glycoside produced by a parent cell, on a per unit volume of cell culture basis.

In some embodiments, the host cell produces an elevated level of a steviol glycoside that is at least about 10%, at least about 15%, at least about 20%, at least about 25%, at least about 30%, at least about 35%, at least about 40%, at least about 45%, at least about 50%, at least about 60%, at least about 70%, at least about 80%, at least about 90%, at least about 2-fold, at least about 2.5-fold, at least about 5-fold, at least about 10-fold, at least about 20-fold, at least about 30-fold, at least about 40-fold, at least about 50-fold, at least about 75-fold, at least about 100-fold, at least about 200-fold, at least about 300-fold, at least about 400-fold, at least about 500-fold, or at least about 1,000-fold, or more, higher than the level of steviol glycoside produced by the parent cell, on a per unit dry cell weight basis.

In some embodiments, the host cell produces an elevated level of a steviol glycoside that is at least about 10%, at least about 15%, at least about 20%, at least about 25%, at least about 30%, at least about 35%, at least about 40%, at least about 45%, at least about 50%, at least about 60%, at least about 70%, at least about 80%, at least about 90%, at least about 2-fold, at least about 2.5-fold, at least about 5-fold, at least about 10-fold, at least about 20-fold, at least about 30-fold, at least about 40-fold, at least about 50-fold, at least about 75-fold, at least about 100-fold, at least about 200-fold, at least about 300-fold, at least about 400-fold, at least about 500-fold, or at least about 1,000-fold, or more, higher than the level of steviol glycoside produced by the parent cell, on a per unit volume of cell culture per unit time basis.

In some embodiments, the host cell produces an elevated level of a steviol glycoside that is at least about 10%, at least about 15%, at least about 20%, at least about 25%, at least about 30%, at least about 35%, at least about 40%, at least about 45%, at least about 50%, at least about 60%, at least about 70%, at least about 80%, at least about 90%, at least about 2-fold, at least about 2.5-fold, at least about 5-fold, at least about 10-fold, at least about 20-fold, at least about 30-fold, at least about 40-fold, at least about 50-fold, at least about 75-fold, at least about 100-fold, at least about 200-fold, at least about 300-fold, at least about 400-fold, at least about 500-fold, or at least about 1,000-fold, or more, higher than the level of steviol glycoside produced by the parent cell, on a per unit dry cell weight per unit time basis.

In most embodiments, the production of the elevated level of steviol glycoside by the host cell is inducible by the presence of an inducing compound. Such a host cell can be manipulated with ease in the absence of the inducing compound. The inducing compound is then added to induce the production of the elevated level of steviol glycoside by the host cell. In other embodiments, production of the elevated level of steviol glycoside by the host cell is inducible by changing culture conditions, such as, for example, the growth temperature, media constituents, and the like.

### Culture Media and Conditions

Materials and methods for the maintenance and growth of microbial cultures are well known to those skilled in the art of microbiology or fermentation science (see, for example, Bailey et al., Biochemical Engineering Fundamentals, second edition, McGraw Hill, New York, 1986). Consideration must be given to appropriate culture medium, pH, temperature, and requirements for aerobic, microaerobic, or anaerobic conditions, depending on the specific requirements of the host cell, the fermentation, and the process.

The methods of producing steviol glycosides provided herein may be performed in a suitable culture medium (e.g., with or without pantothenate supplementation) in a suitable container, including but not limited to a cell culture plate, a microtiter plate, a flask, or a fermentor. Further, the methods can be performed at any scale of fermentation known in the art to support industrial production of microbial products. Any suitable fermentor may be used including a stirred tank fermentor, an airlift fermentor, a bubble fermentor, or any combination thereof. In particular embodiments utilizing Saccharomyces cerevisiae as the host cell, strains can be grown in a fermentor as described in detail by Kosaric, et al, in Ullmann's Encyclopedia of Industrial Chemistry, Sixth Edition, vol. 12, pp. 398-473, Wiley-VCH Verlag Gmbh & Co. KDaA, Weinheim, Germany.

In some embodiments, the culture medium is any culture medium in which a genetically modified microorganism capable of producing a steviol glycoside can subsist. The culture medium may be an aqueous medium comprising assimilable carbon, nitrogen, and phosphate sources. Such a medium can also include appropriate salts, minerals, metals, and other nutrients. The carbon source and each of the essential cell nutrients may be added incrementally or continuously to the fermentation media, and each required nutrient may be maintained at essentially the minimum level needed for efficient assimilation by growing cells, for example, in accordance with a predetermined cell growth curve based on the metabolic or respiratory function of the cells which convert the carbon source to a biomass.

Suitable conditions and suitable media for culturing microorganisms are well known in the art. For example, the suitable medium may be supplemented with one or more additional agents, such as, for example, an inducer (e.g., when one or more nucleotide sequences encoding a gene product are under the control of an inducible promoter), a repressor (e.g., when one or more nucleotide sequences encoding a gene product are under the control of a repressible promoter), or a selection agent (e.g., an antibiotic to select for microorganisms comprising the genetic modifications).

The carbon source may be a monosaccharide (simple sugar), a disaccharide, a polysaccharide, a non-fermentable carbon source, or one or more combinations thereof. Non-limiting examples of suitable monosaccharides include glucose, galactose, mannose, fructose, xylose, ribose, and combinations thereof. Non-limiting examples of suitable disaccharides include sucrose, lactose, maltose, trehalose, cellobiose, and combinations thereof. Non-limiting examples of suitable polysaccharides include starch, glycogen, cellulose, chitin, and combinations thereof. Non-limiting examples of suitable non-fermentable carbon sources include acetate and glycerol.

The concentration of a carbon source, such as glucose, in the culture medium may be sufficient to promote cell growth but is not so high as to repress growth of the microorganism used. Typically, cultures are run with a carbon source, such as glucose, being added at levels to achieve the desired level of growth and biomass. The concentration of a carbon source, such as glucose, in the culture medium may be greater than about 1 g/L, preferably greater than about 2 g/L, and more preferably greater than about 5 g/L. In addition, the concentration of a carbon source, such as glucose, in the culture medium is typically less than about 100 g/L, preferably less than about 50 g/L, and more preferably less than about 20 g/L. It should be noted that references to culture component concentrations can refer to both initial and/or ongoing component concentrations. In some cases, it may be desirable to allow the culture medium to become depleted of a carbon source during culture.

Sources of assimilable nitrogen that can be used in a suitable culture medium include simple nitrogen sources, organic nitrogen sources, and complex nitrogen sources. Such nitrogen sources include anhydrous ammonia, ammonium salts, and substances of animal, vegetable and/or microbial origin. Suitable nitrogen sources include protein hydrolysates, microbial biomass hydrolysates, peptone, yeast extract, ammonium sulfate, urea, and amino acids. Typically, the concentration of the nitrogen sources, in the culture medium is greater than about 0.1 g/L, preferably greater than about 0.25 g/L, and more preferably greater than about 1.0 g/L. Beyond certain concentrations, however, the addition of a nitrogen source to the culture medium is not advantageous for the growth of the microorganisms. As a result, the concentration of the nitrogen sources, in the culture medium is less than about 20 g/L, preferably less than about 10 g/L and more preferably less than about 5 g/L. Further, in some instances it may be desirable to allow the culture medium to become depleted of the nitrogen sources during culture.

The effective culture medium may contain other compounds such as inorganic salts, vitamins, trace metals, or growth promoters. Such other compounds may also be present in carbon, nitrogen, or mineral sources in the effective medium or can be added specifically to the medium.

The culture medium may also contain a suitable phosphate source. Such phosphate sources include both inorganic and organic phosphate sources. Preferred phosphate sources include phosphate salts such as mono or dibasic sodium and potassium phosphates, ammonium phosphate and mixtures thereof. Typically, the concentration of phosphate in the culture medium is greater than about 1.0 g/L, preferably greater than about 2.0 g/L and more preferably greater than about 5.0 g/L. Beyond certain concentrations, however, the addition of phosphate to the culture medium is not advantageous for the growth of the microorganisms. Accordingly, the concentration of phosphate in the culture medium is typically less than about 20 g/L, preferably less than about 15 g/L, and more preferably less than about 10 g/L.

A suitable culture medium can also include a source of magnesium, preferably in the form of a physiologically acceptable salt, such as magnesium sulfate heptahydrate, although other magnesium sources in concentrations that contribute similar amounts of magnesium can be used. Typically, the concentration of magnesium in the culture medium is greater than about 0.5 g/L, preferably greater than about 1.0 g/L, and more preferably greater than about 2.0 g/L. Beyond certain concentrations, however, the addition of magnesium to the culture medium is not advantageous for the growth of the microorganisms. Accordingly, the concentration of magnesium in the culture medium is typically less than about 10 g/L, preferably less than about 5 g/L, and more preferably less than about 3 g/L. Further, in some instances, it may be desirable to allow the culture medium to become depleted of a magnesium source during culture.

The culture medium can also include a biologically acceptable chelating agent, such as the dihydrate of trisodium citrate. In such instance, the concentration of a chelating agent in the culture medium is greater than about 0.2 g/L, preferably greater than about 0.5 g/L, and more preferably greater than about 1 g/L. Beyond certain concentrations, however, the addition of a chelating agent to the culture medium is not advantageous for the growth of the microorganisms. Accordingly, the concentration of a chelating agent in the culture medium is typically less than about 10 g/L, preferably less than about 5 g/L, and more preferably less than about 2 g/L.

The culture medium may also initially include a biologically acceptable acid or base to maintain the desired pH of the culture medium. Biologically acceptable acids include, but are not limited to, hydrochloric acid, sulfuric acid, nitric acid, phosphoric acid, and mixtures thereof. Biologically acceptable bases include, but are not limited to, ammonium hydroxide, sodium hydroxide, potassium hydroxide and mixtures thereof. In some embodiments, the base used is ammonium hydroxide.

The culture medium may also include a biologically acceptable calcium source, including, but not limited to, calcium chloride. Typically, the concentration of the calcium source, such as calcium chloride, dihydrate, in the culture medium is within the range of from about 5 mg/L to about 2000 mg/L, preferably within the range of from about 20 mg/L to about 1000 mg/L, and more preferably in the range of from about 50 mg/L to about 500 mg/L.

The culture medium may also include sodium chloride. Typically, the concentration of sodium chloride in the culture medium is within the range of from about 0.1 g/L to about 5 g/L, preferably within the range of from about 1 g/L to about 4 g/L, and more preferably in the range of from about 2 g/L to about 4 g/L.

The culture medium may also include trace metals. Such trace metals can be added to the culture medium as a stock solution that, for convenience, can be prepared separately from the rest of the culture medium. Typically, the amount of such a trace metals solution added to the culture medium is greater than about 1 ml/L, preferably greater than about 5 mL/L, and more preferably greater than about 10 mL/L. Beyond certain concentrations, however, the addition of a trace metals to the culture medium is not advantageous for the growth of the microorganisms. Accordingly, the amount of such a trace metals solution added to the culture medium is typically less than about 100 mL/L, preferably less than about 50 mL/L, and more preferably less than about 30 mL/L. It should be noted that, in addition to adding trace metals in a stock solution, the individual components can be added separately, each within ranges corresponding independently to the amounts of the components dictated by the above ranges of the trace metals solution.

The culture media may include other vitamins, such as pantothenate, biotin, calcium, pantothenate, inositol, pyridoxine-HCl, and thiamine-HCl. Such vitamins can be added to the culture medium as a stock solution that, for convenience, can be prepared separately from the rest of the culture medium. Beyond certain concentrations, however, the addition of vitamins to the culture medium is not advantageous for the growth of the microorganisms.

The fermentation methods described herein can be performed in conventional culture modes, which include, but are not limited to, batch, fed-batch, cell recycle, continuous, and semi-continuous. In some embodiments, the fermentation is carried out in fed-batch mode. In such a case, some of the components of the medium are depleted during culture, including pantothenate during the production stage of the fermentation. In some embodiments, the culture may be supplemented with relatively high concentrations of such components at the outset, for example, of the production stage, so that growth and/or steviol glycoside production is supported for a period of time before additions are required. The preferred ranges of these components are maintained throughout the culture by making additions as levels are depleted by culture. Levels of components in the culture medium can be monitored by, for example, sampling the culture medium periodically and assaying for concentrations. Alternatively, once a standard culture procedure is developed, additions can be made at timed intervals corresponding to known levels at particular times throughout the culture. As will be recognized by those in the art, the rate of consumption of nutrient increases during culture as the cell density of the medium increases. Moreover, to avoid introduction of foreign microorganisms into the culture medium, addition is performed using aseptic addition methods, as are known in the art. In addition, an anti-foaming agent may be added during the culture.

The temperature of the culture medium can be any temperature suitable for growth of the genetically modified cells and/or production of steviol glycoside. For example, prior to inoculation of the culture medium with an inoculum, the culture medium can be brought to and maintained at a temperature in the range of from about 20° C. to about 45° C., preferably to a temperature in the range of from about 25° C. to about 40° C., and more preferably in the range of from about 28° C. to about 32° C. The pH of the culture medium can be controlled by the addition of acid or base to the culture medium. In such cases, when ammonium hydroxide is used to control pH, it also conveniently serves as a nitrogen source in the culture medium. Preferably, the pH is maintained from about 3.0 to about 8.0, more preferably from about 3.5 to about 7.0, and most preferably from about 4.0 to about 6.5.

The carbon source concentration, such as the glucose concentration, of the culture medium is monitored during culture. Glucose concentration of the culture medium can be monitored using known techniques, such as, for example, use of the glucose oxidase enzyme test or high pressure liquid chromatography, which can be used to monitor glucose concentration in the supernatant, e.g., a cell-free component of the culture medium. The carbon source concentration is typically maintained below the level at which cell growth inhibition occurs. Although such concentration may vary from organism to organism, for glucose as a carbon source, cell growth inhibition occurs at glucose concentrations greater than at about 60 g/L, and can be determined readily by trial. Accordingly, when glucose is used as a carbon source the glucose is preferably fed to the fermentor and maintained below detection limits. Alternatively, the glucose concentration in the culture medium is maintained in the range of from about 1 g/L to about 100 g/L, more preferably in the range of from about 2 g/L to about 50 g/L, and yet more preferably in the range of from about 5 g/L to about 20 g/L. Although the carbon source concentration can be maintained within desired levels by addition of, for example, a substantially pure glucose solution, it is acceptable, and may be preferred, to maintain the carbon source concentration of the culture medium by addition of aliquots of the original culture medium. The use of aliquots of the original culture medium may be desirable because the concentrations of other nutrients in the medium (e.g. the nitrogen and phosphate sources) can be maintained simultaneously. Likewise, the trace metals concentrations can be maintained in the culture medium by addition of aliquots of the trace metals solution.

Other suitable fermentation medium and methods are described in, e.g., WO 2016/196321.

### Fermentation Compositions

Provided herein are fermentation compositions that contain a genetically modified host cell described herein and steviol glycosides produced by the genetically modified host cell. The fermentation compositions may further contain a medium. The fermentation compositions may contain a genetically modified host cell, Reb A, Reb D, and/or Reb M. The fermentation compositions provided herein may contain Reb M as a major component of the steviol glycosides produced by the genetically modified host cell. The fermentation compositions may contain Reb A, Reb D, and Reb M at a ratio of at least 1:7:50. The fermentation compositions may contain Reb A, Reb D, and Reb M at a ratio of at least 1:7:50 to 1:0.5:150. The ratio of Reb A, Reb D, and Reb M may be based on the total content of steviol glycosides that are associated with the genetically modified host cell and the medium. Alternatively, the ratio of Reb A, Reb D, and Reb M may be based on the total content of steviol glycosides in the medium. Further, the ratio of Reb A, Reb D, and Reb M may be based on the total content of steviol glycosides that are associated with the genetically modified host cell.

The fermentation compositions may contain Reb M2 at an undetectable level. In addition, the fermentation compositions may contain non-naturally occurring steviol glycosides at an undetectable level.

### Recovery of Steviol Glycosides

Once the steviol glycoside is produced by the host cell, it may be recovered or isolated for subsequent use using any suitable separation and purification methods known in the art. For example, a clarified aqueous phase containing the steviol glycoside may be separated from the fermentation by centrifugation. Alternatively, a clarified aqueous phase containing the steviol glycoside may be separated from the fermentation by adding a demulsifier into the fermentation reaction. Examples of demulsifiers include flocculants and coagulants.

The steviol glycoside produced in the host cells may be present in the culture supernatant and/or associated with the host cells. Where some of the steviol glycoside is associated with the host cell, the recovery of the steviol glycoside may involve a method of improving the release of the steviol glycosides from the cells. This could take the form of washing the cells with hot water or buffer treatment, with or without a surfactant, and with or without added buffers or salts. The temperature may be any temperature deemed suitable for releasing the steviol glycosides. For example, the temperature may be in a range from 40 to 95° C.; or from 60 to 90° C.; or from 75 to 85° C. Alternatively, the temperature may be 40, 45, 50, 55, 65, 70, 75, 80, 85, 90, or 95° C. Physical or chemical cell disruption may be used to enhance the release of steviol glycosides from the host cell. Alternatively and/or subsequently, the steviol glycoside in the culture medium may be recovered using an isolation unit operations including, solvent extraction, membrane clarification, membrane concentration, adsorption, chromatography, evaporation, chemical derivatization, crystallization, and drying.

### Methods of Making Genetically Modified Cells

Also provided herein are methods for producing a host cell that is genetically engineered to contain one or more of the modifications described above, e.g., one or more heterologous nucleic acids encoding kaurenoic acid hydroxylase and/or biosynthetic pathway enzymes, e.g., for a steviol glycoside compound. Expression of a heterologous enzyme in a host cell can be accomplished by introducing into the host cells a nucleic acid comprising a nucleotide sequence encoding the enzyme under the control of regulatory elements that permit expression in the host cell. The nucleic acid may be an extrachromosomal plasmid, a chromosomal integration vector that can integrate the nucleotide sequence into the chromosome of the host cell, or a linear piece of double stranded DNA that can integrate via homology the nucleotide sequence into the chromosome of the host cell.

Nucleic acids encoding these proteins can be introduced into the host cell by any method known to one of skill in the art (see, e.g., Hinnen et al., (1978) Proc. Natl. Acad. Sci. USA, vol. 75, pp. 1292-1293; Cregg et al., (1985), Mol. Cell. Biol., vol. 5, pp. 3376-3385; Goeddel et al. eds, 1990, Methods in Enzymology, vol. 185, Academic Press, Inc., CA; Krieger, 1990, Gene Transfer and Expression—A Laboratory Manual, Stockton Press, NY; Sambrook et al., 1989, Molecular Cloning—A Laboratory Manual, Cold Spring Harbor Laboratory, NY; and Ausubel et al., eds., Current Edition, Current Protocols in Molecular Biology, Greene Publishing Associates and Wiley Interscience, NY). Exemplary techniques include, spheroplasting, electroporation, PEG 1000 mediated transformation, and lithium acetate or lithium chloride mediated transformation.

The amount of an enzyme in a host cell may be altered by modifying the transcription of the gene that encodes the enzyme. This can be achieved by modifying the copy number of the nucleotide sequence encoding the enzyme (e.g., by using a higher or lower copy number expression vector comprising the nucleotide sequence, or by introducing additional copies of the nucleotide sequence into the genome of the host cell or by deleting or disrupting the nucleotide sequence in the genome of the host cell), by changing the order of coding sequences on a polycistronic mRNA of an operon or breaking up an operon into individual genes each with its own control elements, or by increasing the strength of the promoter or operator to which the nucleotide sequence is operably linked. Alternatively, or in addition, the copy number of an enzyme in a host cell may be altered by modifying the level of translation of an mRNA that encodes the enzyme. This can be achieved by modifying the stability of the mRNA, modifying the sequence of the ribosome binding site, modifying the distance or sequence between the ribosome binding site and the start codon of the enzyme coding sequence, modifying the entire intercistronic region located “upstream of” or adjacent to the 5′ side of the start codon of the enzyme coding region, stabilizing the 3′-end of the mRNA transcript using hairpins and specialized sequences, modifying the codon usage of enzyme, altering expression of rare codon tRNAs used in the biosynthesis of the enzyme, and/or increasing the stability of the enzyme, as, for example, via mutation of its coding sequence.

The activity of an enzyme in a host cell may be altered in a number of ways, including expressing a modified form of the enzyme that exhibits increased or decreased solubility in the host cell, expressing an altered form of the enzyme that lacks a domain through which the activity of the enzyme is inhibited, expressing a modified form of the enzyme that has a higher or lower K_{cat} or a lower or higher Kₘ for the substrate, expressing a modified form of the enzyme that has a higher or lower thermostability, expressing a modified form of the enzyme that has a higher or lower activity at the pH of the cell, expressing a modified form of the enzyme that has a higher or lower accumulation in a subcellular compartment or organelle, expressing a modified form of the enzyme that has increased or decreased ability to insert into or associate with cellular membranes, expressing a modified form of the enzyme that has a higher or lower affinity for accessory proteins needed to carry out a reaction, expressing a modified form of the enzyme that has a higher or lower affinity for necessary cofactors or ligands, expressing a modified form of the enzyme that has an increased or decreased space in the active site (thereby differentially allowing or excluding different substrates for the reaction), or expressing an altered form of the enzyme that is more or less affected by feed-back or feed-forward regulation by another molecule in the pathway.

A nucleic acid used to genetically modify a host cell may contain one or more selectable markers useful for the selection of transformed host cells and for placing selective pressure on the host cell to maintain the foreign DNA.

The selectable marker may be an antibiotic resistance marker. Examples of antibiotic resistance markers include the BLA, NAT1, PAT, AUR1-C, PDR4, SMR1, CAT, mouse dhfr, HPH, DSDA, KAN^{R}, and SH BLE gene products. The BLA gene product from E. coli confers resistance to beta-lactam antibiotics (e.g., narrow-spectrum cephalosporins, cephamycins, and carbapenems (ertapenem), cefamandole, and cefoperazone) and to all the anti-gram-negative-bacterium penicillins except temocillin; the NAT1 gene product from S. noursei confers resistance to nourseothricin; the PAT gene product from S. viridochromogenes Tu94 confers resistance to bialophos; the AUR1-C gene product from Saccharomyces cerevisiae confers resistance to Auerobasidin A (AbA); the PDR4 gene product confers resistance to cerulenin; the SMR1 gene product confers resistance to sulfometuron methyl; the CAT gene product from Tn9 transposon confers resistance to chloramphenicol; the mouse dhfr gene product confers resistance to methotrexate; the HPH gene product of Klebsiella pneumonia confers resistance to Hygromycin B; the DSDA gene product of E. coli allows cells to grow on plates with D-serine as the sole nitrogen source; the KAN^{R} gene of the Tn903 transposon confers resistance to G418; and the SH BLE gene product from Streptoalloteichus hindustanus confers resistance to Zeocin (bleomycin). The antibiotic resistance marker may be deleted after the genetically modified host cell disclosed herein is isolated.

The selectable marker may function by rescue of an auxotrophy (e.g., a nutritional auxotrophy) in the genetically modified microorganism. In auxotrophy, a parent microorganism contains a functional disruption in one or more gene products that function in an amino acid or nucleotide biosynthetic pathway and that renders the parent cell incapable of growing in media without supplementation with one or more nutrients. Such gene products include the HIS3, LEU2, LYS1, LYS2, MET15, TRP1, ADE2, and URA3 gene products in yeast. The auxotrophic phenotype can then be rescued by transforming the parent cell with an expression vector or chromosomal integration construct encoding a functional copy of the disrupted gene product, and the genetically modified host cell generated can be selected for based on the loss of the auxotrophic phenotype of the parent cell. Utilization of the URA3, TRP1, and LYS2 genes as selectable markers has a marked advantage because both positive and negative selections are possible. Positive selection is carried out by auxotrophic complementation of the URA3, TRP1, and LYS2 mutations, whereas negative selection is based on specific inhibitors, i.e., 5-fluoro-orotic acid (FOA), 5-fluoroanthranilic acid, and aminoadipic acid (aAA), respectively, that prevent growth of the prototrophic strains but allows growth of the URA3, TRP1, and LYS2 mutants, respectively. The selectable marker may rescue other non-lethal deficiencies or phenotypes that can be identified by a known selection method.

Described herein are specific genes and proteins useful in the methods, compositions, and host cells of the invention; however, the absolute identity to such genes is not necessary. For example, changes in a particular gene or polynucleotide containing a sequence encoding a polypeptide or enzyme can be performed and screened for activity. Typically, such changes involve conservative mutations and silent mutations. Such modified or mutated polynucleotides and polypeptides can be screened for expression of a functional enzyme using methods known in the art.

Due to the inherent degeneracy of the genetic code, other polynucleotides which encode substantially the same or functionally equivalent polypeptides may also be used to express the enzymes.

It can be advantageous to modify a coding sequence to enhance its expression in a particular host. The genetic code is redundant with 64 possible codons, but most organisms typically use a subset of these codons. The codons that are utilized most often in a species are called optimal codons, and those not utilized very often are classified as rare or low-usage codons. Codons can be substituted to reflect the preferred codon usage of the host, in a process sometimes called “codon optimization” or “controlling for species codon bias.” Codon optimization for other host cells can be readily determined using codon usage tables or can be performed using commercially available software, such as CodonOp from Integrated DNA Technologies.

Optimized coding sequences containing codons preferred by a particular prokaryotic or eukaryotic host (Murray et al., (1989), Nucl Acids Res., vol. 17, pp. 477-508) can be prepared, to increase the rate of translation or to produce recombinant RNA transcripts having desirable properties, such as a longer half-life, as compared with transcripts produced from a non-optimized sequence. Translation stop codons can also be modified to reflect host preference. For example, typical stop codons for S. cerevisiae and mammals are UAA and UGA, respectively. The typical stop codon for monocotyledonous plants is UGA, whereas insects and E. coli commonly use UAA as the stop codon (Dalphin et al., (1996), Nucl Acids Res., vol. 24, pp. 216-218).

Due to the degenerate nature of the genetic code, a variety of DNA molecules differing in their nucleotide sequences may be used to encode a given enzyme of the disclosure. The native DNA sequence encoding the biosynthetic enzymes described above are referenced herein merely to illustrate an embodiment of the disclosure, and the disclosure includes DNA molecules of any sequence that encode the amino acid sequences of the polypeptides and proteins of the enzymes utilized in the methods of the invention. In similar fashion, a polypeptide can typically tolerate one or more amino acid substitutions, deletions, and insertions in its amino acid sequence without loss or significant loss of a desired activity. The invention includes such polypeptides with different amino acid sequences than the specific proteins described herein so long as the modified or variant polypeptides have the enzymatic activity of the reference polypeptide. Furthermore, the amino acid sequences encoded by the DNA sequences shown herein merely illustrate examples of the invention.

In addition, homologs of enzymes useful for the practice of the compositions, methods, or host cells are encompassed by the invention. Two proteins (or a region of the proteins) are considered to be substantially homologous when the amino acid sequences have at least about 30%, 40%, 50%, 60%, 65%, 70%, 75%, 80%, 85%, 90%, 91%, 92%, 93%, 94%, 95%, 96%, 97%, 98%, or 99% identity. To determine the percent identity of two amino acid sequences, or of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). The length of a reference sequence aligned for comparison purposes may be at least 30%, typically at least 40%, more typically at least 50%, even more typically at least 60%, and even more typically at least 70%, 80%, 90%, 100% of the length of the reference sequence. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position (as used herein amino acid or nucleic acid “identity” is equivalent to amino acid or nucleic acid “homology”). The percent identity between the two sequences is a function of the number of identical positions shared by the sequences, taking into account the number of gaps, and the length of each gap, which need to be introduced for optimal alignment of the two sequences.

When “homologous” is used in reference to proteins or peptides, it is recognized that residue positions that are not identical often differ by conservative amino acid substitutions. A “conservative amino acid substitution” is one in which an amino acid residue is substituted by another amino acid residue having a side chain (R group) with similar chemical properties (e.g., charge or hydrophobicity). In general, a conservative amino acid substitution will not substantially change the functional properties of a protein. In cases where two or more amino acid sequences differ from each other by conservative substitutions, the percent sequence identity or degree of homology may be adjusted upwards to correct for the conservative nature of the substitution. Means for making this adjustment are well known to those of skill in the art (See, e.g., Pearson W. R., (1994), Methods in Mol Biol, vol. 25, pp. 365-389).

The following six groups each contain amino acids that are conservative substitutions for one another: 1) Serine(S), Threonine (T); 2) Aspartic Acid (D), Glutamic Acid (E); 3) Asparagine (N), Glutamine (Q); 4) Arginine (R), Lysine (K); 5) Isoleucine (I), Leucine (L), Alanine (A), Valine (V), and 6) Phenylalanine (F), Tyrosine (Y), Tryptophan (W).

Sequence homology for polypeptides, which is also referred to as percent sequence identity, is typically measured using sequence analysis software. A typical algorithm used for comparing a molecule sequence to a database containing a large number of sequences from different organisms is the computer program BLAST. When searching a database containing sequences from a large number of different organisms, it is typical to compare amino acid sequences.

Furthermore, any of the genes encoding the foregoing enzymes or any of the regulatory elements that control or modulate their expression may be optimized by genetic/protein engineering techniques, such as directed evolution or rational mutagenesis. Such action allows those of ordinary skill in the art to optimize the enzymes for expression and activity in yeast.

In addition, genes encoding these enzymes can be identified from other fungal and bacterial species and can be expressed for the modulation of the steviol glycoside pathway. A variety of organisms may serve as sources for these enzymes, including Saccharomyces spp., including S. cerevisiae and S. uvarum, Kluyveromyces spp., including K. thermotolerans, K. lactis, and K. marxianus, Pichia spp., Hansemula spp., including H. polymorpha, Candida spp., Trichosporon spp., Yamadazyma spp., including Y. spp. stipitis, Torulaspora pretoriensis, Issatchenkia orientalis, Schizosaccharomyces spp., including S. pombe, Cryptococcus spp., Aspergillus spp., Neurospora spp., or Ustilago spp. Sources of genes from anaerobic fungi include Piromyces spp., Orpinomyces spp., or Neocallimastix spp. Sources of prokaryotic enzymes that are useful include Escherichia. coli, Zymomonas mobilis, Staphylococcus aureus, Bacillus spp., Clostridium spp., Corynebacterium spp., Pseudomonas spp., Lactococcus spp., Enterobacter spp., and Salmonella spp.

Techniques known to those skilled in the art may be suitable to identify additional homologous genes and enzymes. Generally, analogous genes and/or analogous enzymes can be identified by functional analysis and will have functional similarities. Techniques known to be suitable to identify analogous genes and analogous enzymes include PCR, degenerate PCR, low stringency nucleic acid hybridization, expression cloning, and high through-put screening. For example, to identify homologous or analogous UDP glycosyltransferases, KAH, or any steviol glycoside biosynthetic pathway genes, proteins, or enzymes, techniques may include, but are not limited to, cloning a gene by PCR using primers based on a published sequence of a gene/enzyme of interest, or by degenerate PCR using degenerate primers designed to amplify a conserved region among a gene of interest. Further, one may use techniques to identify homologous or analogous genes, proteins, or enzymes with functional homology or similarity. Techniques include examining a cell or cell culture for the catalytic activity of an enzyme through in vitro enzyme assays for said activity (e.g. as described herein or in Kiritani, K., Branched-Chain Amino Acids Methods Enzymology, 1970), then isolating the enzyme with said activity through purification, determining the protein sequence of the enzyme through techniques such as Edman degradation, design of PCR primers to the likely nucleic acid sequence, amplification of said DNA sequence through PCR, and cloning of said nucleic acid sequence. To identify homologous or similar genes and/or homologous or similar enzymes, analogous genes and/or analogous enzymes or proteins, techniques also include comparison of data concerning a candidate gene or enzyme with databases such as BRENDA, KEGG, or MetaCYC. The candidate gene or enzyme may be identified within the above-mentioned databases in accordance with the teachings herein.

### EXAMPLES

### Example 1: Yeast Transformation Methods

Each DNA construct was integrated into Saccharomyces cerevisiae (CEN.PK113-7D) using standard molecular biology techniques in an optimized lithium acetate transformation. Briefly, cells were grown overnight in yeast extract peptone dextrose (YPD) media at 30° C. with shaking (200 rpm), diluted to an OD₆₀₀ of 0.1 in 100 mL YPD, and grown to an OD₆₀₀ of 0.6-0.8. For each transformation, 5 mL of culture were harvested by centrifugation, washed in 5 mL of sterile water, spun down again, resuspended in 1 mL of 100 mM lithium acetate, and transferred to a microcentrifuge tube. Cells were spun down (13,000×g) for 30 s, the supernatant was removed, and the cells were resuspended in a transformation mix consisting of 240 μL 50% PEG, 36 μL 1 M lithium acetate, 10 μL boiled salmon sperm DNA, and 74 μL of donor DNA. For transformations that require expression of the endonuclease F-CphI, the donor DNA included a plasmid carrying the F-CphI gene expressed under the yeast TDH3 promoter. F-CphI endonuclease expressed in such a manner cuts a specific recognition site engineered in a host strain to facilitate integration of the target gene of interest. Following a heat shock at 42° C. for 40 min, cells were recovered overnight in YPD media before plating on selective media. DNA integration was confirmed by colony PCR with primers specific to the integrations.

### Example 2: Generation of a Base Strain Capable of High Flux to Farnesyl Pyrophosphate (FPP) and the Isoprenoid Farnesene

A farnesene production strain was created from a wild-type Saccharomyces cerevisiae strain (CEN.PK113-7D) by expressing the genes of the mevalonate pathway under the control of native GAL promoters. This strain comprised the following chromosomally integrated mevalonate pathway genes from S. cerevisiae: acetyl-CoA thiolase, HMG-COA synthase, HMG-CoA reductase, mevalonate kinase, phosphomevalonate kinase, mevalonate pyrophosphate decarboxylase, and IPP: DMAPP isomerase. In addition, the strain contained multiple copies of farnesene synthase from Artemisia annua, also under the control of either native GAL1 or GAL10 promoters. All heterologous genes described herein were codon optimized using publicly available or other suitable algorithms. The strain also contained a deletion of the GAL80 gene and the ERG9 gene encoding squalene synthase is downregulated by replacing the native promoter with the promoter of the yeast gene MET3. Examples of methods of creating S. cerevisiae strains with high flux to isoprenoids are described in the U.S. Pat. Nos. 8,415,136 and 8,236,512 which are incorporated herein in their entireties.

### Example 3: Construction of a Series of Strains for Rapid Screening for Novel Kaurenoic Acid Hydroxylase P450 Enzymes

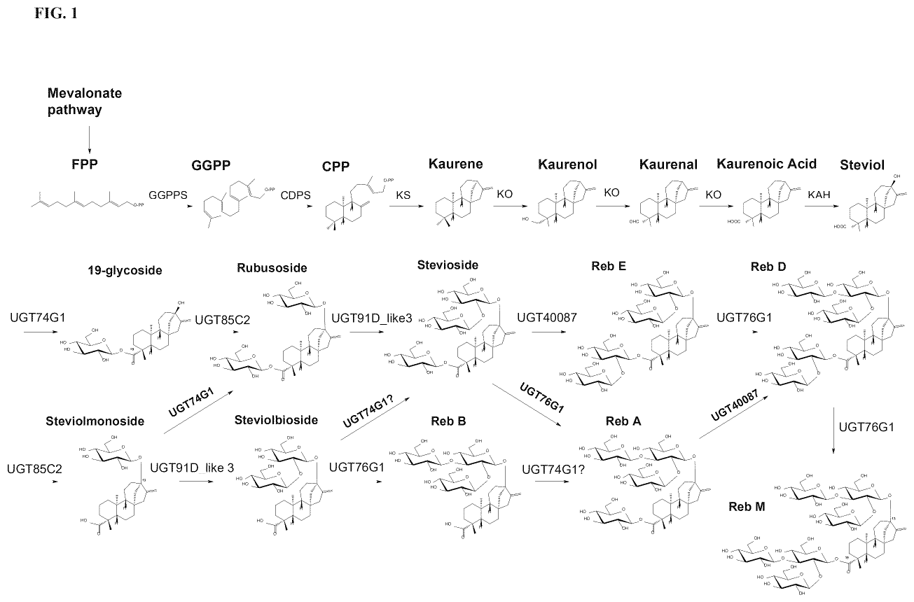

FIG. 1 shows an exemplary biosynthetic pathway from FPP to Reb M with the kaurenoic acid intermediate. The farnesene base strain described above was further engineered to have high flux to the C20 isoprenoid kaurene by integrating six copies of a geranylgeranyl pyrophosphate synthase (GGPPS) into the genome, four copies of a copalyl diphosphate synthase, and four copies of a kaurene synthase. Subsequently, all copies of farnesene synthase were removed from the strain and the strain was confirmed to produce ent-kaurene and no farnesene.

Kaurenoic acid hydroxylase (KAH) is a cytochrome P450 enzyme that catalyzes the oxidation of kaurenoic acid to produce steviol (see FIG. 1) which is necessary to produce Reb M. To screen novel P450 enzymes for KAH activity in vivo in S. cerevisiae, several strains were made that contained all the genes necessary to produce Reb M, except they lacked any copy of a KAH gene. Table 1 lists all Reb M pathway genes and promoters used. The strains containing all genes described in Table 1 primarily produce kaurenoic acid, the substrate for KAH.

TABLE 1
Genes, promoters, and amino acid sequences of
the enzymes used to convert FPP to Reb M.

| Enzyme | SEQ ID | Promoter |
|---|---|---|
| Bt.GGPPS | SEQ ID NO: 7 | PGAL1 |
| Ent-Os.CDPS | SEQ ID NO: 8* | PGAL1 |
| Ent-Pg.KS | SEQ ID NO: 9 | PGAL1 |
| Sr.KO | SEQ ID NO: 6 | PGAL1 |
| At.CPR | SEQ ID NO: 11 | PGAL3 |
| UGT85C2 | SEQ ID NO: 12 | PGAL10 |
| UGT74G1 | SEQ ID NO: 13 | PGAL1 |
| UGT91D_like3 | SEQ ID NO: 14 | PGAL1 |
| UGT76G1 | SEQ ID NO: 15 | PGAL10 |
| UGT40087 | SEQ ID NO: 16 | PGAL1 |

*First 65 amino acids replaced with methionine.

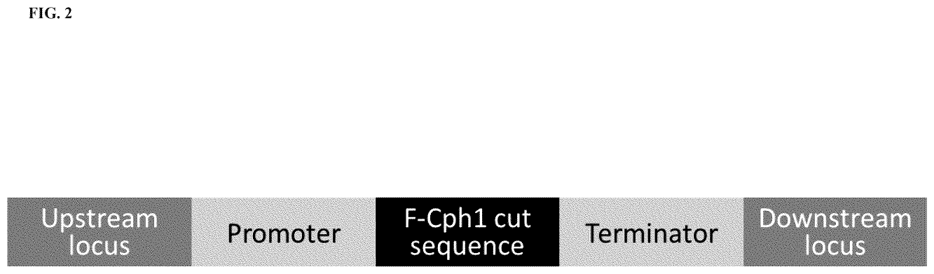

To measure the activity of KAH variants in vivo in S. cerevisiae, initially a first screening strain was constructed that contains all the genes necessary to produce Reb M, except it lacked any copy of a KAH gene (Table 1 and FIG. 1). Instead, it contains a landing pad to allow for the rapid insertion of KAH variants (FIG. 2). The landing pad consists of 500 bp of locus-targeting DNA sequences on either end of the construct to the genomic region upstream and downstream of the yeast locus of choice (Upstream locus and Downstream locus), thereby deleting the locus when the landing pad is integrated into the yeast chromosome. Internally, the landing pad contains a promoter (Promoter) which can be GAL1, GAL3, or any other promoter of yeast GAL regulon and a yeast terminator of choice (Terminator) flanking an endonuclease recognition site (F-CphI). DNA variants of Ro.KAH (SEQ ID NO: 1) were used to transform the strain along with a plasmid expressing endonuclease F-CphI, which cuts the recognition sequence, creating a double strand break at the landing pad, and facilitating homologous recombination of the Ro.KAH DNA variants at the site.

A second screening strain was generated that has the same engineering as the first screening strain except the Sr.KO was replaced with Ps.KO (SEQ ID NO: 10). The Ps.KO enzyme is described in PCT/US2018/046359 (PISUM SATIVUM KAURENE OXIDASE FOR HIGH EFFICIENCY PRODUCTION OF REBAUDIOSIDES filed Aug. 10, 2018) and is significantly more active in converting kaurene to kaurenoic acid (FIG. 1). The second screening strain therefore has a higher carbon flux to kaurenoic acid, the substrate of the KAH P450.

These strains and their modified derivatives (e.g., different GAL promoters and yeast terminators can be used within the landing pad to tune the expression of the KAH genes) are referred to as Reb M producing yeast that lack a functional KAH gene.

### Example 4: Yeast Culturing Conditions

Yeast colonies verified to contain the expected KAH gene were picked into 96-well microtiter plates containing Bird Seed Media (BSM, originally described by van Hoek et al., Biotechnology and Bioengineering 68(5), 2000, pp. 517-523) with 20 g/L sucrose, 37.5 g/L ammonium sulfate, and 1 g/L lysine. Cells were cultured at 30° C. in a high capacity microtiter plate incubator shaking at 1000 rpm and 80% humidity for 3 days until the cultures reached carbon exhaustion. The growth-saturated cultures were subcultured into fresh plates containing BSM with 40 g/L sucrose, 150 g/L ammonium sulfate, and 1 g/L lysine by taking 14.4 μL from the saturated cultures and diluting into 360 μL of fresh media. Cells in the production media were cultured at 30° C. in a high capacity microtiter plate shaker at 1000 rpm and 80% humidity for additional 3 days prior to extraction and analysis.

### Example 5: Whole Cell Broth Sample Preparation Conditions for Analysis of Steviol Glycosides

To extract all steviol glycosides made by cells (see FIG. 1), upon culturing completion, the whole cell broth was diluted with 628 μL of 100% ethanol, sealed with a foil seal, and shaken at 1250 rpm for 30 s. Water (314 μL) was added to each well directly to dilute the extraction, and the plate was briefly centrifuged to pellet solids. Ethanol: water mixture (198 μL of 50:50) containing 0.48 mg/L rebaudioside N (used as an internal standard) was dispensed to a new 250 μL assay plate and 2 μL of the culture/ethanol mixture was added to the assay plate. A foil seal was applied to the plate for analysis.

### Example 6: Analytical Methods

Samples derived from yeast producing steviol glycosides (Example 5) were routinely analyzed using mass spectrometer (Agilent 6470-QQQ) with a RapidFire 365 system autosampler with C8 cartridge. Steviol glycosides and an undesirable impurity abbreviated as C20H32O4+1Glc were measured in the assay.

TABLE 2
RapidFire 365 system configuration.

| | |
|---|---|
| Pump 1, Line A: 2 mM ammonium | 100% A, 1.5 mL/min |
| formate in water | |
| Pump 2, Line A: 35% | 100% A, 1.5 mL/min |
| acetonitrile in water | |
| Pump 3, Line A: 80% | 100% A, 0.8 mL/min |
| acetonitrile in water | |
| State 1: Aspirate | 600 ms |
| State 2: Load/wash | 3000 ms |
| State 3: Extra wash | 1500 ms |
| State 4: Elute | 5000 ms |
| State 5: Reequilibrate | 1000 ms |

TABLE 3
6470-QQQ MS method configuration.

| Ion source | AJS ESI | |
|---|---|---|
| Time filtering peak width | 0.02 | min |
| Stop time | No limit/as pump | |
| Scan type | MRM | |
| Diverter valve | To MS | |
| Delta EMV | (+)0/(−)300 | |
| Ion mode (polarity) | Negative | |
| Gas temperature | 250° | C. |
| Gas flow | 11 | L/min |
| Nebulizer | 30 | psi |
| Sheath gas temperature | 350° | C. |
| Sheath gas flow | 11 | L/min |
| Negative capillary voltage | 2500 | V |

The mass spectrometer was operated in negative ion multiple reaction monitoring (MRM) mode. Each steviol glycoside was identified from precursor ion mass and MRM transition (Table 4). The fragmentation at labile carboxylic ester linkage at the C19 allowed for distinction between regioisomers Reb A and Reb E while no distinction can be made between rubusoside and steviolbioside (steviol+2Glc) or stevioside and Reb B (steviol+3Glc) using this method.

TABLE 4
Steviol glycosides and masses for corresponding
precursor and product ions.

| Compound | Precursor ion (Da) | Product ion (Da) |
|---|---|---|
| steviol + 1Glc | 479.265 | 317.212 |
| steviol + 2Glc | 641.318 | 479.265 |
| steviol + 3Glc | 803.371 | 641.318 |
| Reb A | 965.424 | 803.371 |
| Reb E | 965.424 | 641.318 |
| steviol + 5Glc | 1127.476 | 803.371 |
| steviol + 6Glc | 1289.529 | 803.371 |
| C20H32O4 + 1Glc | 543.281 (formate) | 335.222 |

The peak areas from a chromatogram from a mass spectrometer were used to generate the calibration curve using authentic standards. The molar ratios of relevant compounds were determined by quantifying the amount in moles of each compound through external calibration using an authentic standard, and then taking the appropriate ratios. Due to the absence of a purified authentic standard for C20H32O4+1Glc, the relative production of C20H32O4+1Glc in different yeast strains was evaluated from the corresponding peak area only.

To determine specific steviol glycosides and to evaluate the presence of new side products, selected samples were also analyzed using ultra-high-performance liquid chromatography (UHPLC) on Thermo Fisher Scientific Vanquish UHPLC system equipped with Acquity UPLC BEH C18 column (15 cm, 2.1 mm, 1.7 μm, 130 Å; part #186002353) (Table 5). Dual detection was performed using Vanquish charged aerosol detector (CAD) (Table 6) and Thermo Fisher Scientific Q-Exactive Orbitrap mass spectrometer (Table 7) with post-column flow split 5:1 (5 to CAD and 1 to MS) using Restek binary fixed-flow splitter.

TABLE 5
Vanquish UHPLC chromatographic conditions.

| Mobile phase A | 0.1% formic acid in water | | |
|---|---|---|---|
| Mobile phase B | 0.1% formic acid in acetonitrile | | |
| Flow rate | 0.4 mL/min | | |
| Column temperature | 50° C. | | |
| Pre-heater temperature | 50° C. | | |
| | Time (min) | A % | B % |
| Gradient | 0 | 80 | 20 |
| | 2 | 80 | 20 |
| | 28 | 54 | 46 |
| | 28.1 | 5 | 95 |
| | 32 | 5 | 95 |
| | 32.5 | 80 | 20 |
| | 36 | 80 | 20 |

TABLE 6
Vanquish CAD detector configuration.

| Power function | 1.00 | |
|---|---|---|
| Data collection rate | 2 | Hz |
| Filter | 3.6 | |
| Gas regulation mode | Analytical | |
| Evaporator temperature | 35° | C. |

TABLE 7
Q-Exactive Orbitrap MS method configuration.
Ion source conditions:

| Ion source | ESI | |
|---|---|---|
| Sheath gas flow rate | 40 | |
| Auxiliary gas flow rate | 15 | |
| Sweep gas flow rate | 2 | |
| Spray voltage | 3500 | V |
| Capillary temperature | 375° | C. |
| S-Lens RF level | 60.0 | |
| Auxiliary gas heater temperature | 400° | C. |

Scan settings:

| General | Runtime | 0 to 36 min |
|---|---|---|
| | Polarity | Negative |
| | Default charge state | 1 |
| | Inclusion | On |
| | Exclusion | On |
| | Scan type | Full MS - ddMS² |
| Full MS | Resolution | 70,000 |

| AGC target | 1e6 | |
|---|---|---|
| Maximum IT | 50 | ms |

| | Scan range | 300 to 2000 m/z |
|---|---|---|
| | Spectrum data type | Centroid |
| ddMS² | Resolution | 35,000 |

| AGC target | 1e5 | |
|---|---|---|
| Maximum IT | 50 | ms |
| Loop count | 10 | |
| TopN | 10 | |
| Isolation window | 2.0 | m/z |

| | | |
|---|---|---|
| | Stepped (N)CE | nce: 10, 30, 40 |
| dd Settings | Minimum AGC target | 8.00e3 |

| Charge exclusion | >3 | |
|---|---|---|
| Exclude isotopes | On | |
| Dynamic exclusion | 4.0 | s |

| | |
|---|---|
| If idle . . . | Pick others |

The peak identities were assigned to steviol glycosides and C20H32O4+1Glc based on retention time determined from an authentic standard, molecular ion, and MS fragmentation pattern (Table 8).

TABLE 8
Steviol glycosides, their retention times and precursor ion.

| | Retention time | |
|---|---|---|
| Compound | (min) | Precursor ion (Da) |
| Steviol | 27.8 | 317.212 |
| Steviolmonoside | 20.6 | 479.265 |
| 19-glycoside | 19.4 | 479.265 |
| Steviolbioside | 17.5 | 641.318 |
| Rubusoside | 15.5 | 641.318 |
| Reb B | 17.6 | 803.371 |
| Stevioside | 12.7 | 803.371 |
| Reb E | 7.4 | 965.424 |
| Reb A | 12.7 | 965.424 |
| Reb D | 8.0 | 1127.476 |
| Reb M | 8.8 | 1289.529 |
| C20H32O4 + 1Glc | 9.5 | 543.281 (formate) |

### Example 7: KAH from Rubus occidentalis is a Novel P450 Enzyme with KAH Activity

A previously identified KAH enzyme (Rs.KAH; SEQ ID NO: 3) from the plant Rubus suavissimus (also known as Chinese blackberry or Chinese sweet leaf) was used as a query to search the genome of the related plant black raspberry (Rubus occidentalis). The whole-genome assembly of R. occidentalis is publicly available at (https://www.rosaceae.org/organisms/Rubus/occidentalis). Using the default tBLASTn feature on the website and the Rs.KAH amino acid sequence as the query sequence, a putative KAH ortholog was identified encoded by five exons on chromosome 3. The protein sequence of the putative Ro.KAH was deduced by translating the DNA coding sequence of the five exons into a single protein sequence (SEQ ID NO: 1). A yeast-codon optimized DNA sequence (SEQ ID NO: 2) for the putative Ro.KAH protein was generated, and the corresponding synthetic DNA sequence was assembled.

The yeast codon-optimized Ro.KAH gene was PCR-amplified using primers designed to have 40 bp of flanking homology to the promoter and the yeast terminator DNA sequences in the landing pad (see FIG. 2). These flanks were added to the ends of the amplified gene to facilitate homologous recombination into the landing pad at the specific locus of the host strain. Additional other KAH genes used as experimental controls were also amplified with primers containing the same 40 bp of flanking sequences in the landing pad. Each KAH gene was transformed individually as a single copy into a Reb M-screening strain described above (Example 3) and screened for the ability to produce steviol glycosides when expressed in vivo. The host strains described in Example 3 are unable to produce steviol glycosides as they lack any KAH gene. Therefore, in order to produce steviol glycosides, the gene product introduced into the transformants must have KAH activity and be able to convert kaurenoic acid to steviol for further glycosylation by the UGT genes of host yeast.

To determine in vivo KAH activity in a host strain, steviol glycosides in the whole cell broth produced by a strain were extracted and measured via mass spectrometry (Examples 4-6). The sum of all the steviol glycosides was calculated (in μM) and reported as total steviol glycosides (TSG). The activity of individual KAH variants was calculated by normalizing TSG measurements for a strain containing a particular KAH enzyme to that of a strain containing the wild-type Rs.KAH enzyme.

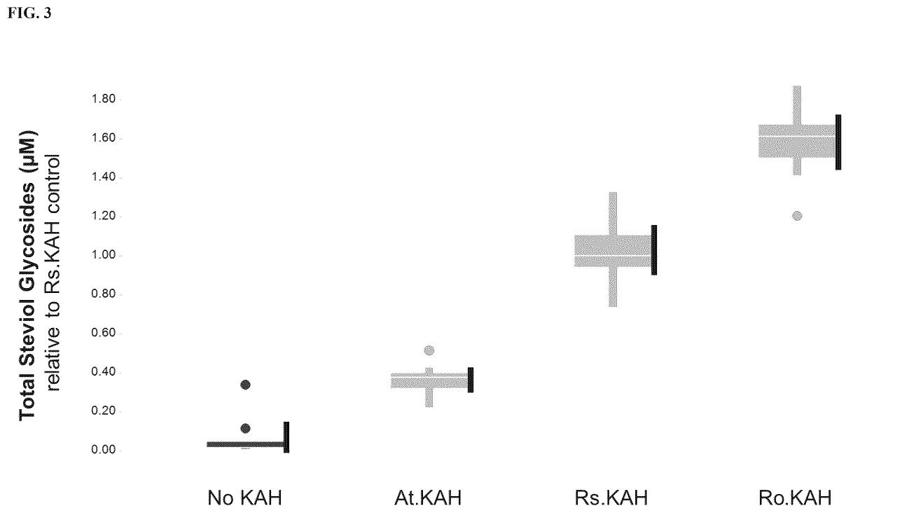

Overexpression of the putative Ro.KAH sequence in a host strain confirmed that the protein product of this gene encodes a KAH activity (FIG. 3). As expected, a host strain with no KAH gene does not make any steviol glycosides. Expression of the previously identified genes encoding At.KAH (SEQ ID NO: 4) or Rs.KAH (SEQ ID NO: 3) led to the production of steviol glycosides (FIG. 3). The activity of Ro.KAH measured in such a manner was higher than KAH activity of either At.KAH or Rs.KAH (4- and 1.5-fold higher, respectively).

### Example 8: Evolution of Wild-Type Ro.KAH Via Site-Directed Saturation Mutagenesis

In this example, activity data is provided for wild-type Ro.KAH and specific mutations that improve Ro.KAH activity when expressed in S. cerevisiae host for production of steviol glycosides including Reb M.

Each amino acid residue in Ro.KAH was mutated using degenerate codons producing two sets of libraries. Degenerate codons consisted of either NDT combined with VHG (first set) or NNY (second set), where N stands for any nucleotide adenine, thymine, guanine, and cytosine; D stands for adenine, guanine, and thymine; T stands for thymine; V stands for adenine, cytosine, and guanine; H stands for adenine, cytosine, and thymine; G stands for guanine; and Y stands for cytosine and guanine. The degenerate codon NDT encodes 12 different amino acids (R, N, D, C, G, H, I, L, F, S, Y, and V) whereas the degenerate codon VHG encodes 9 different amino acids (A, E, K, L, M, P, Q, T, and V) in equal fractions. When VHG and NDT are mixed in a ratio of 3:4, all amino acids except for tryptophan are represented in a roughly equimolar amount. The degenerate codon NNY encodes 15 different amino acids (A, C, D, F, G, H, I, L, N, P, R, S, T, V, and Y). Each library was constructed via PCR using primers designed to introduce a degenerate codon so that each PCR product contains a mixture of gene variants where 19 (first set) or 15 (second set) possible different amino acids are encoded at a specific position corresponding to a single protein residue. In each PCR product, the pool of Ro.KAH gene variants are flanked on both ends by 40 bp of sequences homologous to promoter and terminator regions of the landing pad in a host strain, as described in previous examples.

Each variant pool, representing changes at a single amino acid in Ro.KAH was transformed independently into a host yeast and screened for the effect of mutations on KAH activity as described above. For Tier 1 screening, 24 colonies were chosen per site to screen, roughly representing a 1.4× sampling rate of the library. Every amino acid in the wild-type Ro.KAH sequence (SEQ ID NO: 1) was subjected to mutagenesis and screening as described.

The amount of steviol produced by KAH is deduced by summing all of the steviol glycosides (total steviol glycosides, or TSG; in μM). The effect of a particular mutation was calculated by comparing TSG produced by a strain containing a mutant amino acid to TSG produced by a strain containing the wild-type Ro.KAH protein. Upon finding mutations in Ro.KAH that increased activity of the enzyme in vivo, a Tier 2 screen was performed with higher replication (N≥8) to confirm the improvement in steviol production, using the same calculations as above. A mutation was considered to improve Ro.KAH activity if the median amount of steviol glycosides produced by the mutant was at least one standard deviation above the median amount of steviol glycosides produced by the wild-type Ro.KAH protein.

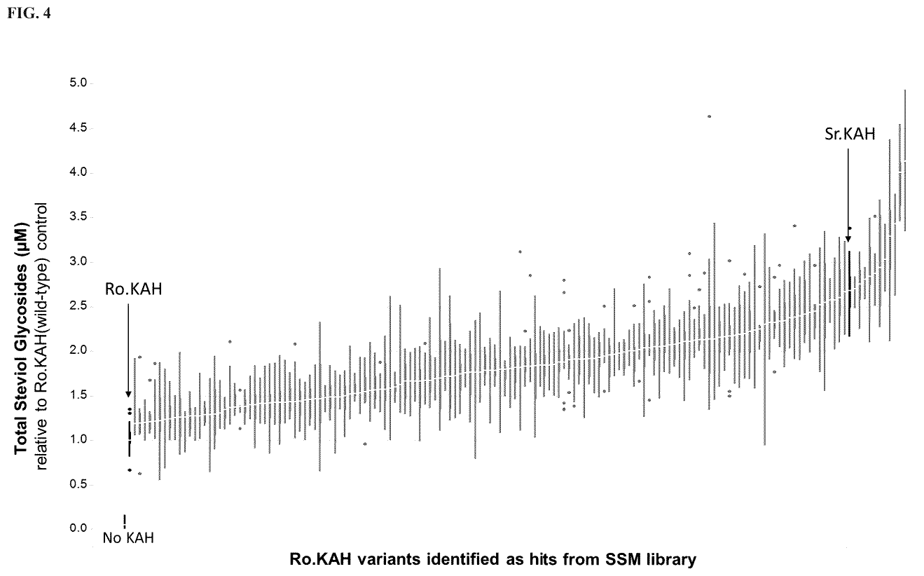

In total, 154 unique mutations were found by screening two sets of libraries described above that improved Ro.KAH activity more than one standard deviation above the activity of the wild-type enzyme (FIG. 4). Table 9 lists the median fold improvement for each mutation over wild-type Ro.KAH. The activity of wild-type Sr.KAH is included in FIG. 4 and Table 9 for reference. Note, that the top six mutants of Ro.KAH (S452D, I104A, V340S, F229Y, A297Y, A297F) have activity improved one standard deviation or more than that of wild-type Sr.KAH.

TABLE 9
Ro.KAH alleles that increase activity of wild-type Ro.KAH
measured as total steviol glycosides more than one standard
deviation. Associated amino acid change and fold improvement
over wild-type Ro.KAH are listed.

| | Fold improvement over wild- |
|---|---|
| Ro.KAH sequence variation | type Ro.KAH |
| No KAH | 0.05 |
| wild-type Ro.KAH | 1.00 |
| K69R | 1.19 |
| V343G | 1.20 |
| T403V | 1.20 |
| H491P | 1.20 |
| P72D | 1.21 |
| L64D | 1.22 |
| Q84C | 1.24 |
| L64G | 1.24 |
| E206D | 1.26 |
| Y238C | 1.26 |
| A210G | 1.26 |
| L64N | 1.26 |
| I237C | 1.27 |
| L11V | 1.27 |
| N207F | 1.28 |
| M73G | 1.28 |
| W8G | 1.28 |
| E60R | 1.31 |
| Y55S | 1.34 |
| N475G | 1.34 |
| D292P | 1.37 |
| P161C | 1.37 |
| K267D | 1.38 |
| L485F | 1.38 |
| A396F | 1.40 |
| R507A | 1.40 |
| P72T | 1.41 |
| I132G | 1.41 |
| N61P | 1.42 |
| K119V | 1.43 |
| T220E | 1.43 |
| P72G | 1.43 |
| Q513R | 1.44 |
| S133G | 1.45 |
| Y506V | 1.46 |
| K69P | 1.47 |
| E60G | 1.47 |
| K224C | 1.48 |
| M73H | 1.49 |
| H379G | 1.49 |
| P72C | 1.49 |
| K314P | 1.50 |
| W202A | 1.52 |
| G466F | 1.52 |
| N49A | 1.52 |
| S339G | 1.53 |
| N160D | 1.54 |
| T216G | 1.56 |
| D102Y | 1.56 |
| F246G | 1.56 |
| M58P | 1.58 |
| T220R | 1.59 |
| R458D | 1.60 |
| M58G | 1.61 |
| A68I | 1.62 |
| S70P | 1.66 |
| F88V | 1.66 |
| T240D | 1.67 |
| L205I | 1.67 |
| K167G | 1.67 |
| L232M | 1.67 |
| S62R | 1.69 |
| G56D | 1.70 |
| Q244G | 1.70 |
| A242D | 1.72 |
| N49R | 1.72 |
| Q513G | 1.73 |
| W29T | 1.75 |
| L303D | 1.76 |
| T378D | 1.77 |
| I508L | 1.78 |
| W202Q | 1.79 |
| S505R | 1.79 |
| R233C | 1.80 |
| I104D | 1.80 |
| M258G | 1.81 |
| K69G | 1.81 |
| F88D | 1.82 |
| F88S | 1.82 |
| A217V | 1.83 |
| E230C | 1.83 |
| R507G | 1.83 |
| G466S | 1.85 |
| G56S | 1.87 |
| E230G | 1.88 |
| Y55G | 1.89 |
| A503C | 1.89 |
| S460I | 1.90 |
| I129R | 1.91 |
| S245G | 1.91 |
| F246S | 1.92 |
| Q84L | 1.92 |
| S133R | 1.92 |
| T509V | 1.93 |
| R507E | 1.94 |
| R233T | 1.94 |
| V30F | 1.96 |
| A68G | 1.96 |
| G56N | 1.97 |
| T162G | 1.98 |
| A68P | 1.99 |
| S165D | 2.01 |
| K119Y | 2.02 |
| W29C | 2.04 |
| S165P | 2.05 |
| W29V | 2.05 |
| I284G | 2.06 |
| A217L | 2.06 |
| Q335V | 2.08 |
| L65S | 2.09 |
| F53R | 2.09 |
| Y55P | 2.09 |
| W202V | 2.10 |
| K224V | 2.12 |
| W29A | 2.13 |
| H164G | 2.13 |
| Q244D | 2.13 |
| K291C | 2.14 |
| L65G | 2.15 |
| K167S | 2.16 |
| C327I | 2.16 |
| K291S | 2.16 |
| D57G | 2.17 |
| K167H | 2.17 |
| N160T | 2.19 |
| W202C | 2.23 |
| A242G | 2.25 |
| F88R | 2.32 |
| I104N | 2.32 |
| G466D | 2.34 |
| N475D | 2.34 |
| K119S | 2.35 |
| T123D | 2.37 |
| T216A | 2.38 |
| S339A | 2.42 |
| P161D | 2.45 |
| I104R | 2.48 |
| L54G | 2.51 |
| M171F | 2.55 |
| L232Y | 2.57 |
| D293C | 2.57 |
| V340A | 2.60 |
| T162A | 2.63 |
| Sr.KAH | 2.68 |
| A297V | 2.70 |
| I104H | 2.75 |
| F332L | 2.80 |
| A236R | 2.87 |
| K224I | 2.93 |
| S452D | 2.94 |
| I104A | 3.03 |
| V340S | 3.21 |
| F229Y | 3.43 |
| A297Y | 3.93 |
| A297F | 4.13 |

### Example 9: Evolution of Ro.KAH Via Combinatorial Mutagenesis (24 Amino Acid Residues Targeted for Mutagenesis in a Full-Factorial Fashion)

Two sets of 12 mutations were selected from the unique site-directed saturation mutagenesis hits described in Example 8 to build two full factorial combination libraries. The first combinatorial library contained mutations L54G, Y55P, F88R, I104A, K119S, T123D, P161D, T162A, S165D, M171F, T216A, K224I, and the second library contained mutations F229Y, L232Y, A236R, I284G, K291C, D293C, A297Y, F332L, S339A, V340A, S452D, L485F. The libraries were designed to create all possible combinations among the 12 mutations to find the combination that leads to the highest activity of Ro.KAH in vivo. The genes were assembled from a mixture of PCR-amplified fragments containing desired mutations. Each fragment contained overlapping homology on the ends of each piece so that the pieces overlapped in sequence; assembling all the pieces together in vitro using PCR reconstituted a full length KAH allele. The terminal 5′ and 3′ pieces also had homology to the promoter and terminator of the landing pad sequence in Reb M producing yeast that lack a functional KAH gene. The assembled full-length library genes were transformed into yeast.

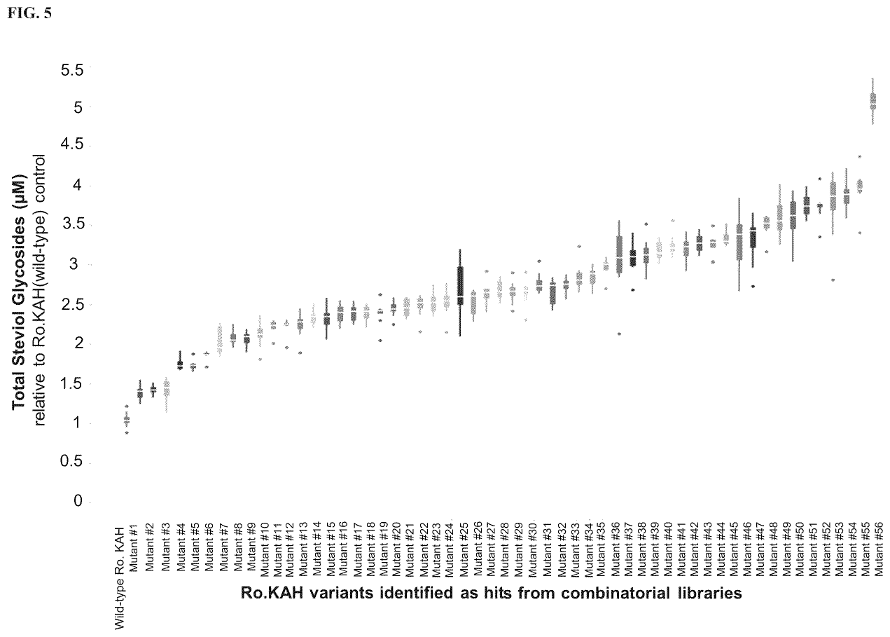

The Tier 1 combinatorial library DNA was screened in the Reb M producing yeast at a 0.9× coverage. The effect of particular mutation combinations was calculated by comparing TSG produced by a strain containing multiple mutant amino acids to TSG produced by a strain containing the wild-type Ro.KAH protein as described above (Example 8). The 56 highest performing KAH alleles identified from the combinatorial library screen were promoted to Tier 2 confirmation at N=8 replication. Most of these variants produced at least 1.4-fold higher TSG comparing to wild-type Ro.KAH, with the top variant resulting in 5.02-fold improved production (FIG. 5 and Table 10). Interestingly, most of the improved variants contained only two or three mutations per variant, only five variants contained four mutations, and there were no improved variants identified that contained more than four mutations (Table 10). The best combination of amino acid substitutions comprised only two mutations: F332L and S452D.

TABLE 10
Improved alleles of Ro.KAH, fold improvement in TSG over wild-
type Ro.KAH activity, and the associated amino acid changes.

| | Fold improvement | |
|---|---|---|
| | over wild-type | |
| Ro.KAH allele | Ro.KAH | Genotype of the mutant |
| wild-type Ro.KAH | 1.00 | |
| mutant #1 | 1.40 | L232Y/S339A |
| mutant #2 | 1.42 | A297Y/L485F |
| mutant #3 | 1.44 | S339A/V340A/L485F |
| mutant #4 | 1.72 | K291C/V340A |
| mutant #5 | 1.73 | K291C/V340A/L485F |
| mutant #6 | 1.86 | I284G/K291C/S339A/V340A |
| mutant #7 | 1.95 | I284S/K291C/V340A/S452D |
| mutant #8 | 2.04 | K291C/S339A/V340A/S452D |
| mutant #9 | 2.09 | L54G/F88R/K224I |
| mutant #10 | 2.12 | F332L/S339A/S452D |
| mutant #11 | 2.23 | I284G/A297Y |
| mutant #12 | 2.25 | I284G/S339A/V340A |
| mutant #13 | 2.27 | F332L/S452D/L485F |
| mutant #14 | 2.34 | F88R/T216A |
| mutant #15 | 2.34 | K291C/S339A |
| mutant #16 | 2.40 | L54G/T216A |
| mutant #17 | 2.41 | Y55P/F88R |
| mutant #18 | 2.41 | I104A/K119S/T162A/K224I |
| mutant #19 | 2.42 | L54G/F88R/T123D/T162A |
| mutant #20 | 2.44 | I284G/K291C/V340A |
| mutant #21 | 2.46 | L54G/K119S/T123D |
| mutant #22 | 2.52 | I104A/M171F/T216A |
| mutant #23 | 2.52 | L54G/K224I |
| mutant #24 | 2.54 | F332L/S339A/V340A |
| mutant #25 | 2.59 | I284G/K291C/S339A |
| mutant #26 | 2.61 | F88R/M171F |
| mutant #27 | 2.65 | S452D/L485F |
| mutant #28 | 2.65 | I284G/V340A |
| mutant #29 | 2.66 | L54G/Y55P/T162A |
| mutant #30 | 2.67 | P161D/T162A |
| mutant #31 | 2.73 | L54G/Y55P/T123D |
| mutant #32 | 2.74 | Y55P/F88R/K119S |
| mutant #33 | 2.75 | S339A/V340A |
| mutant #34 | 2.81 | I284G/K291C/F332L |
| mutant #35 | 2.89 | K119S/T123D/T216A |
| mutant #36 | 3.00 | I104A/T162A/K224I |
| mutant #37 | 3.09 | I104A/K119S/M171F |
| mutant #38 | 3.10 | T216A/K224I |
| mutant #39 | 3.12 | F229Y/K291C |
| mutant #40 | 3.15 | M171F/T216A |
| mutant #41 | 3.21 | I104A/K119S |
| mutant #42 | 3.23 | Y55P/T123D |
| mutant #43 | 3.27 | I104A/T162A/M171F |
| mutant #44 | 3.28 | K291C/F332L/V340A |
| mutant #45 | 3.29 | T123D/T162A/K224I |
| mutant #46 | 3.38 | F332L/V340A |
| mutant #47 | 3.43 | L54G/T123D |
| mutant #48 | 3.52 | K119S/T216A |
| mutant #49 | 3.55 | T162A/K224I |
| mutant #50 | 3.62 | I284G/K291C/S452D |
| mutant #51 | 3.73 | K119S/M171F |
| mutant #52 | 3.77 | T123D/T162A/T216A |
| mutant #53 | 3.86 | L54G/F88R/T123D |
| mutant #54 | 3.89 | K291C/S452D |
| mutant #55 | 3.96 | T123D/T216A |
| mutant #56 | 5.02 | F332L/S452D |

### Example 10: Identification of Undesirable Impurity C20H32O4+1Glc Derived from Side-Product of Ro.KAH Activity on Kaurenoic Acid

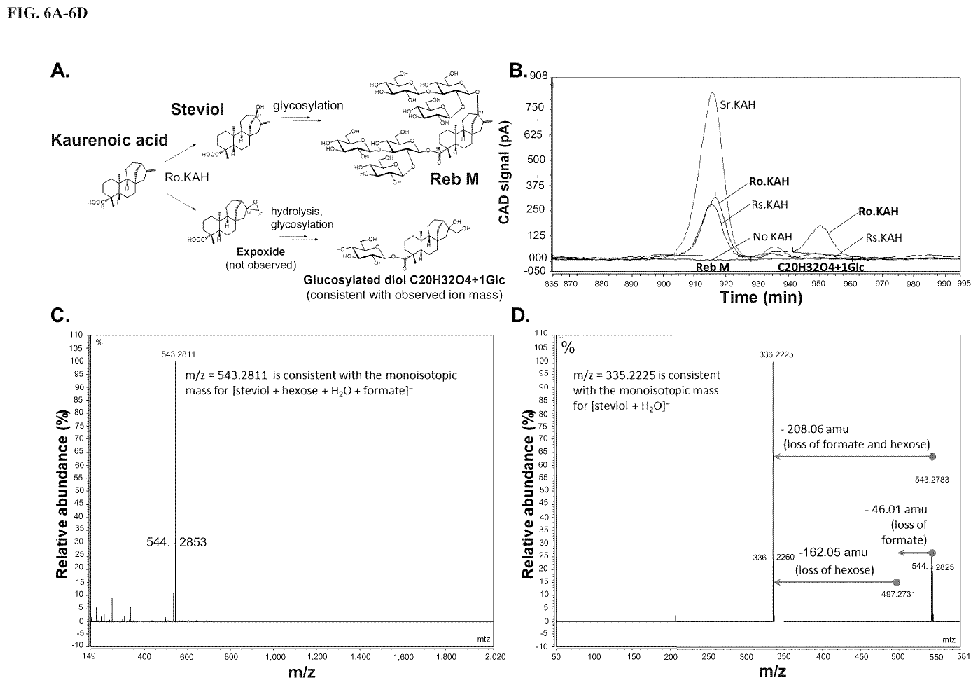

Samples derived from yeast expressing Ro.KAH were analyzed for production of additional metabolites that are not part of the Reb M pathway depicted in FIG. 1 using UHPLC equipped with tandem CAD and Orbitrap-MS detectors (see Example 6 for analysis details). A comparison of CAD chromatograms of strains expressing either Ro.KAH, Sr.KAH, Rs.KAH, or no KAH identified one peak that is specific to Ro.KAH (FIG. 6B). Further inspection of the high-resolution ion mass and MS/MS fragmentation pattern established that this peak of interest has a molecular formula C26H4209 consistent with singly glycosylated kaurenoic acid+oxygen+water (FIGS. 6C and 6D). A small peak with the same retention time in CAD chromatogram is also detectable for Rs.KAH-expressing Reb M producer, however the abundance is too low for MS characterization.

Based on the known scope of reactivity of the cytochrome P450 superfamily, it appears that Ro.KAH can convert kaurenoic acid to either of two products, steviol or an epoxide (FIG. 6A). The epoxide can then spontaneously hydrolyze to diol and be glycosylated at the carboxylate site (C19) by one of the glycosyltransferases of the Reb M pathway to generate a glucosylated diol product consistent with the ion mass detected for the new peak. Although stereocenters already present in the kaurenoic acid core are likely preserved in this diol named C20H32O4+1Glc, the stereochemistry at C16 cannot be determined using the methods used in this study.

C20H32O4+1Glc (the structure as depicted in FIG. 6A) with defined S configuration at C16 has been previously isolated from several plants. Most notably it has been identified as a tasteless minor component of sweet leaves of R. suavissimus, the source of Rs.KAH (Ohtani et al., Phytochemistry 31 (5), 1992, pp. 1553-1559) and named suavioside E. Stevia phlebophylla A. Gray, a rare Mexican species, appears to produce the same compound (Ceunen et al., Carbohydrate Research 379, 2013, pp. 1-6) as well as several Chinese medicinal plant species of genera Aster (Cheng et al., Phytochemistry 33 (5), 1993, pp. 1181-1183; Tan et al., Journal of Natural Products 56 (11), 1993, pp. 1917-1922; Tan et al., Magnetic Resonance in Chemistry 33 (9), 1995, pp. 749-754) and several species of African plants of genera Cussonia (Harinantenaina et al., Chemical & Pharmaceutical Bulletin 50 (2), 2002, pp. 268-271; Harinantenaina et al., Chemical & Pharmaceutical Bulletin 50 (8), 2002, pp. 1122-1123; Harinantenaina et al., Phytochemistry 61 (4), 2002, pp. 367-372).

C20H32O4+1Glc (the structure as depicted in FIG. 6A) with defined R configuration at C16, often referred to as paniculoside IV, has also been isolated from a number of different plants including but not limited to Cussonia species (Harinantenaina et al., Chemical & Pharmaceutical Bulletin 50 (2), 2002, pp. 268-271; Harinantenaina et al., Chemical & Pharmaceutical Bulletin 50 (8), 2002, pp. 1122-1123; Harinantenaina et al., Phytochemistry 61(4), 2002, pp. 367-372), Pulicaria species (Darwish et al., Alexandria Journal of Pharmaceutical Sciences 15(1), 2001, pp. 21-24; Rasool et al., Natural Product Communications 3(2), 2008, pp. 141-144; Rasool et al., Natural Product Communications 8(6), 2013, pp. 757-759), Acanthopanax koreanum (Cai et al., Archives of Pharmacal Research 26(9), 2003, pp. 731-734; Cai et al., Phytotherapy Research 18(8), 2004, pp. 677-680) and several others.

The reports of prior isolation and characterization of the plant-derived natural products described above provide support for the proposed structure for C20H32O4+1Glc shown in FIG. 6A, but do not definitively confirm it. Nevertheless, the production of C20H32O4+1Glc only in the presence of Ro.KAH confirms the formation by this enzyme of a side product in addition to steviol. This necessitated the evolution of Ro.KAH enzyme variants with improved product specificity in addition to improved overall activity, i.e. increased formation of steviol with reduced absolute and relative amounts of the undesirable side product.

### Example 11: Improvement of Ro.KAH A297Y Activity and Product Specificity Via Site-Directed Saturation Mutagenesis Targeting 46 Amino Acid Residues

As described in Example 10, yeast expressing Ro.KAH produces not only Reb M and other minor steviol glycosides (as Reb M pathway intermediates) but also an undesirable side product C20H32O4+1Glc at the molar ratio of approximately 1 to 2 (C20H32O4+1Glc to Reb M). Thus, improvements in both activity and product specificity of Ro.KAH were targeted with the libraries described in the following examples.

To further improve activity and product specificity of Ro.KAH, another round of site-directed saturation mutagenesis was applied to one of the top SSM variants described in Example 8 (Ro.KAH A297Y). Each of 46 selected amino acids (F128, 1129, 1132, L136, L140, A141, T142, G143, L144, A145, N146, Y238, K241, I243, I273, I277, K328, L329, F330, Y331, F332, A333, G334, Q335, E336, T337, T338, A396, V397, 1398, E399, L400, P401, P463, F464, G465, G466, G467, P468, R469, 1470, 1472, G473, Y506, I508, and T509) in the Ro.KAH A297Y sequence was mutated to 20 different amino acids (A, R, N, D, C, E, Q, G, H, I, L, K, M, F, P, S, T, W, Y, and V) that are encoded by the degenerate codon NNS where N stands for any nucleotide adenine, thymine, guanine and cytosine and S stands for guanine and cytosine. The NNS library of Ro.KAH A297Y gene variants was constructed via PCR using primers containing an NNS degenerate codon at the desired position as described in Example 8 and used to transform Reb M producing yeast that lack a functional KAH gene.

The in vivo KAH activity of the NNS library mutants was measured in a Tier 1 screen. The effect of a particular mutation (in addition to A297Y) was calculated by comparing Reb M titers produced by a mutant strain to Reb M produced by a strain containing the wild-type Ro.KAH protein. Because of the high efficiency of the downstream glycosyltransferases in the Reb M pathway, Reb M constitutes the majority of TSG while other steviol glycosides are present only in minute amounts. Using relative Reb M titer as a measure of KAH in vivo activity simplifies data analysis and produces the same ranking of the variants as relative TSG measurement. The in vivo KAH product specificity of the NNS library mutants was also measured in a Tier 1 screen relative to wild-type Ro.KAH using the peak area from an MS chromatogram for the side-product C20H32O4+1Glc and the titer of Reb M (μM). The fold change in product specificity was estimated by calculating the ratio of Reb M (μM) produced by the mutant relative to the wild-type Ro.KAH to the side-product C20H32O4+1Glc produced by the mutant relative to the wild-type Ro.KAH:

$$specificity change = \frac{{[ Reb M titer ]}_{mutant} {/ [ Reb M titer ]}_{wild - type}}{\begin{matrix}{[ C 20 H 32 O 4 + 1 Glc peak area ]}_{mutant} / \\ {[ C 20 H 32 O 4 + 1 Glc peak area ]}_{wild - type}\end{matrix}} .$$

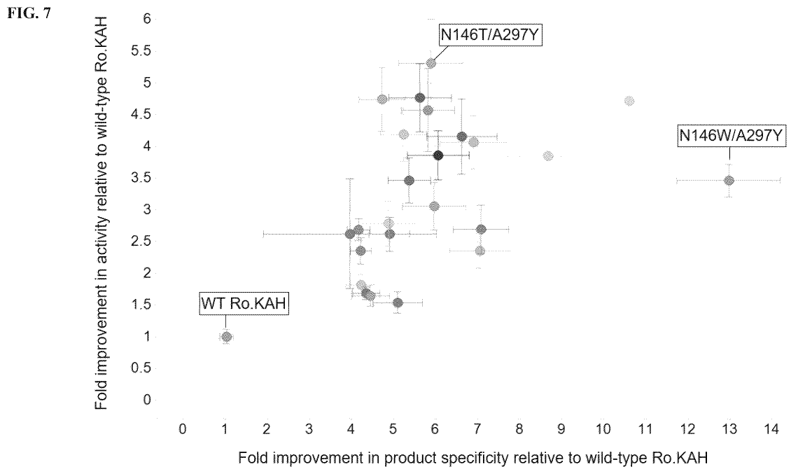

Thirty-nine isolates were screened per mutated position in the protein sequence giving approximately a 1.2× coverage at N=1 for each unique variant. Upon finding mutations in combination with Ro.KAH A297Y that appeared to increase activity and product specificity of the enzyme in vivo, a Tier 2 screen was performed with higher replication (N=8) of strains containing a specific mutant of interest to confirm the improvement, using calculations as described above. The resulting activity and product specificity of an NNS-derived mutant Ro.KAH allele is reported in fold improvement over wild-type Ro.KAH in FIG. 7 and Table 11.

TABLE 11
Improved alleles of Ro.KAH A297Y: the associated amino acid changes and
fold improvement over wild-type Ro.KAH activity and product specificity.

| | | Fold improvement in product |
|---|---|---|
| Ro.KAH sequence variations | Fold improvement in activity | specificity over wild-type |
| compared to Ro.KAH A297Y | over wild-type Ro.KAH | Ro.KAH |
| wild-type Ro.KAH | 1.00 | 1.00 |
| Ro.KAH A297Y | 3.70 | 3.07 |
| N146P | 1.54 | 5.10 |
| N146E | 1.65 | 4.45 |
| A141F | 1.68 | 4.35 |
| A141L | 1.81 | 4.23 |
| G466M | 2.35 | 4.22 |
| A141I | 2.36 | 7.06 |
| T142C | 2.62 | 4.90 |
| N146D | 2.69 | 7.08 |
| T142R | 2.69 | 4.17 |
| T142V | 2.78 | 4.89 |
| N146Y | 3.06 | 5.97 |
| N146W | 3.46 | 12.97 |
| T142D | 3.46 | 5.37 |
| N146L | 3.85 | 8.68 |
| T142A | 3.86 | 6.06 |
| N146Q | 4.06 | 6.90 |
| T142G | 4.15 | 6.62 |
| T142S | 4.19 | 5.24 |
| A141V | 4.57 | 5.82 |
| N146M | 4.72 | 10.61 |
| N146S | 4.74 | 4.72 |
| N146V | 4.77 | 5.63 |
| N146T | 5.31 | 5.89 |

Twenty-three mutations in combination with A297Y were identified that lead to improvements over wild-type Ro.KAH in both activity and product specificity, with most of the improved variants arising from amino acid substitutions at the positions 141, 142, and 146. The best activity mutant from this site-directed saturation mutagenesis NNS library (N146T/A297Y) provides 5.2-fold improvement of in vivo KAH activity comparing to wild-type Ro.KAH. The best specificity mutant (N146W/A297Y) provides 12.97-fold improvement in KAH product specificity comparing to wild-type Ro.KAH. Interestingly, the highest improvements in both activity and specificity resulted from mutagenesis at the same position, N146, but from different amino acid substitutions (T and W, respectively).

### Example 12: Improvement of Ro.KAH A297Y Activity and Product Specificity Via Combinatorial Site-Directed Saturation Mutagenesis

To further improve activity and product specificity of Ro.KAH, combinatorial site-directed saturation mutagenesis was applied to one of the top SSM variants described in Example 8 (Ro.KAH A297Y). In this type of mutagenesis, also known as combinatorial active-site saturation test (CAST; Reetz et al., Angewandte Chemie International Edition English 44(27), 2005, pp. 4192-4196), two amino acid residues that are spatially close are mutated at the same time which allows for potential synergistic effects arising from side-chain orientation. Accordingly, ten libraries were created where two amino acid residues in Ro.KAH A297Y mutant were mutated at the same time: F128 and F129, L144 and A145, A145 and N146, K328 and F332, F330 and G334, F332 and T337, A396 and V397, V397 and I398, R507 and I508, I508 and T509. Each amino acid residue was mutated to 12 different amino acids (R, N, D, C, G, H, I, L, F, S, Y, and V) that are encoded by the degenerate codon NDT where N stands for any nucleotide adenine, thymine, guanine, and cytosine, D stands for adenine, guanine, and thymine; T stands for thymine. The NDT library of Ro.KAH A297Y gene variants was constructed via PCR using primers containing an NDT degenerate codon at the desired position as described in Example 8 and used to transform Reb M producing yeast that lack a functional KAH gene.

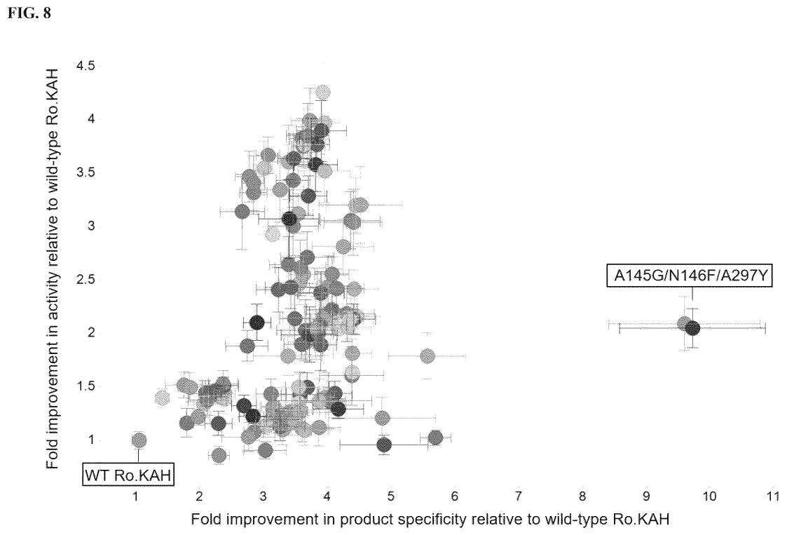

The changes in the in vivo KAH activity and product specificity of the mutants were measured as described in Example 11. Thirty-nine isolates were screened per mutated position in the protein sequence giving approximately a 2× coverage at N=1 for each unique variant in Tier 1. Upon finding mutations in combination with Ro.KAH A297Y that appeared to increase activity and product specificity of the enzyme in vivo, a Tier 2 screen was performed with higher replication (N=8) of strains containing a specific mutant of interest to confirm the improvement. The resulting activity and product specificity of a mutant Ro.KAH allele is reported in fold increase over wild-type Ro.KAH in FIG. 8. Table 12 lists a subset of library hits depicted in FIG. 8, specifically those with two (not just one) amino acid changes simultaneously introduced into Ro.KAH A297Y.

TABLE 12
Improved alleles of Ro.KAH A297Y: the associated amino acid changes and
fold improvement over wild-type Ro.KAH activity and product specificity.

| | | Fold improvement in product |
|---|---|---|
| Ro.KAH sequence variations | Fold improvement in activity | specificity over wild-type |
| compared to Ro.KAH A297Y | over wild-type Ro.KAH | Ro.KAH |
| wild-type Ro.KAH | 1.00 | 1.00 |
| Ro.KAH A297Y | 3.70 | 3.07 |
| F128V/I129G | 0.86 | 2.30 |
| A145G/N146L | 0.96 | 4.89 |
| F128C/I129V | 1.10 | 3.30 |
| A145S/N146I | 1.21 | 4.86 |
| F128V/I129C | 1.22 | 2.83 |
| F128L/I129V | 1.26 | 3.53 |
| I508V/T509V | 1.29 | 4.17 |
| F128V/I129V | 1.32 | 2.69 |
| F128Y/I129C | 1.43 | 3.56 |
| F128V/I129R | 1.51 | 2.37 |
| V397I/I398V | 1.52 | 1.75 |
| A145G/N146V | 1.60 | 4.38 |
| V397D/I398V | 1.77 | 2.84 |
| A145S/N146V | 1.81 | 4.39 |
| A145G/N146F | 2.09 | 9.60 |
| A145G/N146S | 2.16 | 4.46 |
| F129Y/I129R | 2.41 | 3.23 |
| F128Y/I129V | 2.61 | 3.57 |
| R507N/I508V | 3.06 | 4.36 |
| R507C/I508V | 3.20 | 4.44 |

A number of variants with product specificity improvements relative to wild-type Ro.KAH were identified from this combinatorial library, with the variant A145G/N146F/A297Y resulting in 9.6-fold specificity improvement relative to wild-type Ro.KAH. Improvements in specificity came at a cost of sacrificing overall activity: none of the variants with two amino acid changes relative to parent Ro.KAH A297Y and only a handful with a single amino acid change had better activity than parent enzyme. This is not surprising when more than one amino acid residue is mutated simultaneously.

### Example 13: Evolution of Ro.KAH N146T/A297Y Via Full Site-Directed Saturation Mutagenesis

To further improve the activity of Ro.KAH N146T/A297Y (top activity variant, Example 11) another round of site-directed saturation mutagenesis was applied to isolate mutant variants with even higher activity in converting kaurenoic acid to steviol (FIG. 1). Each amino acid residue in Ro.KAH N146T/A297Y sequence was mutated to 15 different amino acids (A, C, D, F, G, H, I, L, N, P, R, S [encoded by two codons], T, V, and Y) that are encoded by the degenerate codon NNT where N stands for any nucleotide adenine, thymine, guanine, and cytosine and T stands for thymine. The NNT library of Ro.KAH N146T/A297Y was constructed via PCR using primers containing an NNT degenerate codon at the desired position as described in Example 8 and used to transform Reb M producing yeast that lack a functional KAH gene.

The relative in vivo KAH activity and product specificity of the Ro.KAH N146T/A297Y mutants were assessed as described in Example 11. Thirteen isolates were screened per mutated position in the protein sequence giving approximately a 0.8× coverage at N=1 for each unique variant in Tier 1. Upon finding mutations in Ro.KAH N146T/A297Y that appeared to increase activity of the enzyme in vivo, a Tier 2 screen was performed with higher replication (N=8) of strains containing a specific mutant of interest to confirm the improvement. Variants with improved activity confirmed in Tier 2 were subjected to Tier 3 validation, in which DNA sequences of the mutant variants were PCR-amplified from a Reb M-producing strains with improved performance using primers containing homology to the promoter and terminator sequences, and used to transform Reb M-producing yeast lacking a KAH gene. Tier 3 eliminates false-positives in Tier 1 and Tier 2 arising due to an unintended, randomly introduced, mutation elsewhere in yeast genome. The resulting activity and product specificity of the NNT-derived mutant Ro.KAH N146T/A297Y alleles are reported in fold increase over Ro.KAH N146T/A297Y in Table 13.

TABLE 13
Improved alleles of Ro.KAH N146T/A297Y: the associated amino acid changes and
fold improvement over Ro.KAH N146T/A297Y activity and product specificity.

| Ro.KAH sequence variations | | Fold improvement in product |
|---|---|---|
| compared to Ro.KAH | Fold improvement in activity | specificity over Ro.KAH |
| N146T/A297Y | over Ro.KAH N146T/A297Y | N146T/A297Y |
| Ro.KAH N146T/A297Y | 1.00 | 1.00 |
| S505I | 1.18 | 1.04 |
| W52T | 1.21 | 0.97 |
| S457G | 1.23 | 1.06 |
| Q84R | 1.23 | 1.20 |
| G466A | 1.25 | 1.20 |

Five mutations were identified that lead to improvements in activity, ranging from 1.18 to 1.25-fold over Ro.KAH N146T/A297Y. Four of these resulted in modest specificity improvements as well, with the top two variants, Q84R/N146T/A297Y and N146T/A297Y/G466A, resulting in 1.2-fold specificity improvement.

### Example 14: Evolution of Ro.KAH N146W/A297Y Via Full Site-Directed Saturation Mutagenesis

To further improve the activity of Ro.KAH N146W/A297Y (top specificity variant, Example 11) another round of site-directed saturation mutagenesis was applied to isolate mutant variants with even higher activity in converting kaurenoic acid to steviol (FIG. 1). Each amino acid residue in Ro.KAH N146W/A297Y sequence was mutated to 15 different amino acids encoded by the degenerate codon NNT as described in Example 13 and resulting mutants were tested in the Reb M producing yeast that lack a functional KAH gene.

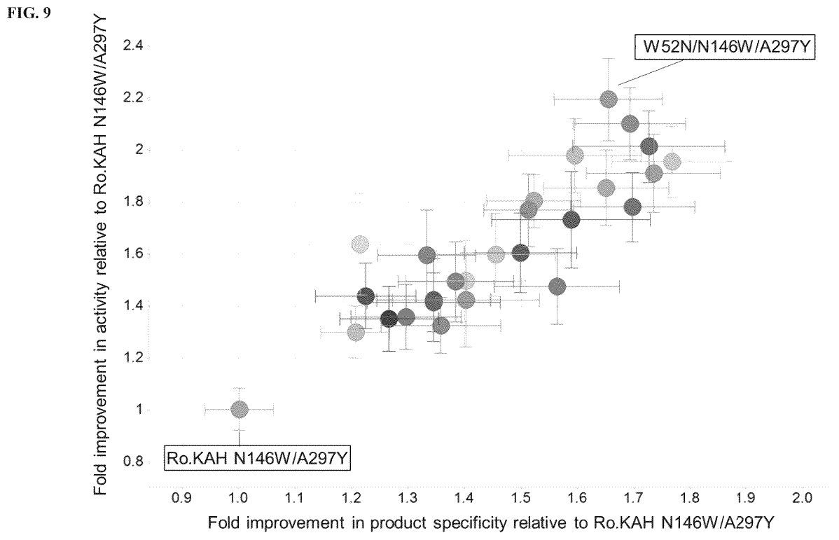

The relative in vivo KAH activity and product specificity of the Ro.KAH N146T/A297Y mutants were assessed as described in Example 11. Library coverage and tiered screening were performed as described in Example 13. The resulting activity and product specificity of an NNT-derived mutant Ro.KAH N146W/A297Y alleles are reported in fold increase over Ro.KAH N146W/A297Y in FIG. 9 and Table 14.

TABLE 14
Improved alleles of Ro.KAH N146W/A297Y: the associated amino acid changes and
fold improvement over Ro.KAH N146W/A297Y activity and product specificity.

| Ro.KAH sequence variations | | Fold improvement in product |
|---|---|---|
| compared to Ro.KAH | Fold improvement in activity | specificity over Ro.KAH |
| N146W/A297Y | over Ro.KAH N146W/A297Y | N146W/A297Y |
| Ro.KAH N146W/A297Y | 1.00 | 1.00 |
| A236S | 1.30 | 1.21 |
| V9S | 1.33 | 1.26 |
| G466F | 1.33 | 1.36 |
| T283D | 1.36 | 1.30 |
| T142G | 1.39 | 1.34 |
| T425V | 1.42 | 1.35 |
| L459C | 1.42 | 1.35 |
| T283A | 1.46 | 1.24 |
| T283G | 1.49 | 1.38 |
| S460V | 1.50 | 1.42 |
| S133G | 1.51 | 1.57 |
| I129V | 1.60 | 1.33 |
| W52G | 1.60 | 1.46 |
| S505I | 1.62 | 1.51 |
| I243T | 1.64 | 1.21 |
| V340S | 1.73 | 1.59 |
| S460C | 1.77 | 1.51 |
| S452D | 1.79 | 1.70 |
| L118I | 1.82 | 1.53 |
| S505V | 1.85 | 1.65 |
| T123D | 1.91 | 1.73 |
| W52C | 1.92 | 1.57 |
| S460I | 1.95 | 1.77 |
| S457G | 2.01 | 1.71 |
| W52R | 2.10 | 1.69 |
| W52N | 2.19 | 1.66 |

Twenty-six mutations were identified that lead to improvements in activity, ranging from 1.3 to 2.2-fold over Ro.KAH N146W/A297Y. The same twenty-six mutations also resulted in specificity improvements over Ro.KAH N146W/A297Y, with the top variant, 5 Ro.KAH N146W/A297Y/S460I, resulting in 1.8-fold specificity improvement.

### Example 15: Evolution of Ro.KAH N146T/A297Y/G466A Via Site-Directed Saturation Mutagenesis and Mutations Consolidation

In attempts to further improve the activity of the top mutant variant with highest KAH activity, Ro.KAH N146T/A297Y/G466A (Example 13, Table 13), was subjected to yet another round of site-directed saturation mutagenesis targeting 68 amino acids many of which were shown to improve activity in the earlier Examples (W52, Q84, T123, I129, T142, T216, 1265, K267, T283, F332, V340, S452, S457, S460, S505): W52, Q84, T123, I129, T142, R170, M171, L172, P173, S174, F175, H176, Q177, S178, C179, T180, T216, 1265, N266, K267, E268, I269, K270, G271, L272, I273, I277, I278, K279, R280, E281, H282, T283, I284, K285, A286, G287, E288, F332, V340, Q409, L410, G411, K412, F413, S414, L415, P416, E417, G418, V419, E420, V421, R422, L423, P424, T425, L426, L427, 1428, H429, H430, D431, K432, S452, S457, S460, S505). Each of these 68 amino acids in Ro.KAH N146T/A297Y/G466A sequence was mutated to 15 different amino acids encoded by the degenerate codon NNT as described in Example 13 and resulting mutants were tested in the Reb M producing yeast that lack a functional KAH gene. The relative in vivo KAH activity and product specificity of the Ro.KAH N146T/A297Y mutants were assessed as described in Example 11. Library coverage and tiered screening was as described in Example 13.

Very few hits were identified in this screen and even these had only modest activity improvements. Thus, the new top activity hit Ro.KAH T142G/N146T/A297Y/G466A produced less than 10% more Reb M than the library parent, Ro.KAH N146T/A297Y/G466A (Table 15). Interestingly, Ro.KAH T142G/N146T/A297Y/G466A variant also had the best product specificity in this set: 1.7-fold improved over library parent. The specific mutation T142G had also been identified as beneficial for both activity and product specificity in the earlier rounds of engineering yielding activity improvements that diminished with each subsequent round of engineering: over 4-fold in Ro.KAH A297Y (Example 11, Table 11) and of 40% in Ro.KAH N146W/A297Y (Example 14, Table 14).

To explore if mutation T142G is beneficial in other backgrounds, it was tested in combination with other top beneficial mutations, W52T and G466A. Briefly, gene sequences for the variants Ro.KAH W52T/T142G/N146T/A297Y and Ro.KAH W52T/T142G/N146T/A297Y/G466A were assembled from a mixture of PCR-amplified fragments containing desired mutations. Each fragment contained overlapping homology on the ends of each piece so that the pieces overlapped in sequence; assembling all the pieces together in vitro using PCR reconstituted a full length KAH allele. The terminal 5′ and 3′ pieces also had homology to the promoter and terminator of the landing pad sequence. The assembled full-length genes were transformed into the Reb M producing yeast that lack a functional KAH gene and their relative in vivo KAH activity and product specificity were assessed as described in Example 11. The resulting activity and product specificity of the Ro.KAH variants are reported in fold increase over Ro.KAH N146T/A297Y/G466A in Table 15.

TABLE 15
Improved alleles of Ro.KAH N146T/A297Y: the associated amino acid changes and fold
improvement over Ro.KAH N146T/A297Y/G466A activity and product specificity.

| Ro.KAH sequence variations | Fold improvement in activity | Fold improvement in product |
|---|---|---|
| compared to Ro.KAH | over Ro.KAH | specificity over Ro.KAH |
| N146T/A297Y | N146T/A297Y/G466A | N146T/A297Y/G466A |
| G466A | 1.00 | 1.00 |
| W52T | 1.04 | 0.86 |
| T142G/G466A | 1.08 | 1.71 |
| W52T/T142G | 0.88 | 1.17 |
| W52T/T142G/G466A | 1.12 | 1.45 |

No dramatic improvements over the triple mutant Ro.KAH N146T/A297Y/G466A were detected for the new quadruple and quintuple mutants of Ro.KAH. With returns diminishing with each subsequent round of engineering, a local minimum may have been reached. Interestingly, incorporating only the T142G mutation into the Ro.KAH W52T/N146T/A297Y sequence led to reduction in activity but improvement in product specificity, whereas incorporating T142G and G466A improved both activity and specificity (Table 15).

### Example 16: N-Terminal Domain Swaps to Improve In Vivo Activity of Ro.KAH Variants

This example provides modified kaurenoic acid hydroxylase polypeptides with substituted N-terminal domains that show improved activity.

Kaurenoic acid hydroxylase is a cytochrome P450 enzyme. Most eukaryotic P450s are membrane-bound proteins, and the high-level domain structure of membrane-associated cytochrome P450 enzymes is highly conserved. Plant cytochrome P450 enzymes are incorporated into the endoplasmic reticulum (ER) with an N-terminal polypeptide chain of roughly 30-50 amino acids that mediates membrane targeting. The catalytic domain of the P450 enzymes face the cytoplasmic side of the endoplasmic reticulum. It was demonstrated that swapping the ER-associated N-terminus of Sr.KAH with other N-terminal transmembrane domains, for example that of cytochrome P450 reductase (CPR) from Artemisia annua (Aa.CPR), results in improved KAH enzymatic activity. See PCT/US2019/056153. A similar approach was tested for Ro.KAH variants as described below.

The transmembrane domain in Ro.KAH was predicted using a TMHMM server (available at http://www.cbs.dtu_dk/services/TMHMM/). It was then replaced with the first 66 amino acids of Aa.CPR (SEQ ID NO: 22). Truncations of different lengths (removing from 23 to 50 amino acids from the N-terminus of Ro.KAH) were explored while the sequence of Aa.CPR fused to the N-terminus of these truncations remained constant. Gene sequences for the chimeric proteins were assembled from a mixture of PCR-amplified fragments of Aa.CPR and Ro.KAH variants containing overlapping homology and were transformed into the Reb M producing yeast that lack a functional KAH gene. The in vivo KAH activity of chimeric proteins was assessed as described in Example 11 and is reported in Table 16 as fold increase in Reb M titers over the corresponding Ro.KAH variant without any domain swap. It was established that truncating the first 25 amino acids of Ro.KAH resulted in the highest activity of N-terminal fusion for each of the variants tested; thus only the data for these chimeras are shown in Table 16.

TABLE 16
Alleles of Aa.CPR-Ro.KAH fusions resulting from N-terminal domain swaps and fold
improvement in Reb M titers over Ro.KAH parent sequence without domain swap.

| Ro.KAH allele Aa.CPR(1a:66a)-Ro.KAH | | Fold improvement in activity |
|---|---|---|
| variant(26a:523a) | SEQ ID NO | over Ro.KAH variant (1a-523a) |
| N146T/A297Y/G466A | 17 | 1.09 |
| W52T/N146T/A297Y | 18 | 1.17 |
| T142G/N146T/A297Y/G466A | 19 | 1.08 |
| W52T/T142G/N146T/A297Y | 20 | 1.11 |
| W52T/T142G/N146T/A297Y/G466A | 21 | 1.15 |

For all the Ro.KAH mutants tested, N-terminal domain swaps with Aa.CPR resulted in Ro.KAH variants with 10-17% improvements in KAH activity. The product profile (relative titers of Reb M and C20H32O4+1Glc) of chimeric proteins was unchanged with N-terminal domain swap (data not shown). This is expected, as partitioning to the side product is likely determined by amino acids of the catalytic domain of P450 and not by the membrane-targeting domain. Although modest, the increase in KAH activity resulting from N-terminal domain swap appears to be additive with the activity improvements resulting from point mutations within Ro.KAH sequence providing yet another lever for activity improvement.

All publications, patents and patent applications cited in this specification are herein incorporated by reference as if each individual publication or patent application were specifically and individually indicated to be incorporated by reference. Although the foregoing invention has been described in some detail by way of illustration and example for purposes of clarity of understanding, it will be readily apparent to those of ordinary skill in the art in light of the teachings of this invention that certain changes and modifications may be made thereto without departing from the spirit or scope of the appended claims.

1. A kaurenoic acid hydroxylase variant polypeptide comprising the amino acid sequence of SEQ ID NO: 1, wherein the sequence further comprises one or more amino acid substitutions comprising one or more of V9S, W52C, W52G, W52N, W52R, W52T, L118I, I129V, T142G, A145G, N146F N146T, N146W, A236S, I243T, T283A, T283D, T283G, T425V, S457G, L459C, S460C, S460V, G466A, S505I, and S505V.

2-7. (canceled)

8. The variant polypeptide of claim 1, wherein the one or more amino acid substitutions comprise N146T/A297Y/G466A; W52T/N146T/A297Y; T142G/N146T/A297Y/G466A; W52T/T142G/N146T/A297Y; or W52T/T142G/N146T/A297Y/G466A.

9. The variant polypeptide of claim 1, wherein the one or more amino acid substitutions comprise W52T/T142G/G466A; A145G/N146F/A297Y; N146W/A297Y/S460I; W52N/N146W/A297Y; N146W/A297Y; or Q84R/N146T/A297Y.

10. The variant polypeptide of claim 1, wherein the amino acids encoding a N-terminal signal sequence in SEQ ID NO: 1 are replaced with the amino acids of an N-terminal signal sequence of a heterologous plant p450 polypeptide.

11. The variant polypeptide of claim 1, wherein amino acids 1-25 of SEQ ID NO: 1 are replaced with the amino acid sequence of SEQ ID NO: 22.

12. (canceled)

13. The variant polypeptide of claim 11, comprising the amino acid sequence selected from SEQ ID NO: 17, SEQ ID NO: 18, SEQ ID NO: 19, SEQ ID NO: 20, and SEQ ID NO: 21.

14. A nucleic acid encoding the variant polypeptide of claim 1.

15. A host cell comprising the polypeptide of claim 1.

16. The host cell of claim 15, wherein the host cell is capable of producing one or more steviol glycosides.

17. The host cell of claim 16, wherein the one or more steviol glycosides are selected from RebA, RebB, RebD, RebE, and RebM.

18. (canceled)

19. The host cell of claim 15, further comprising one or more nucleic acids encoding one or more enzymes of a pathway for making a steviol glycoside.

20. The host cell of claim 19, further comprising a nucleic acid encoding a geranylgeranyl diphosphate synthase, a nucleic acid encoding a geranylgeranyl diphosphate synthase, a nucleic acid encoding a copalyl diphosphate synthase, a nucleic acid encoding an ent-kaurene synthase, a nucleic acid encoding a kaurene oxidase, a nucleic acid encoding a cytochrome P450 reductase, and/or a nucleic acid encoding one or more uridine 5′-diphosphate-dependent glycosyltransferases.

21-26. (canceled)

27. The host cell of claim 15, wherein the cell is selected from a bacterial cell, a yeast cell, an algal cell, an insect cell, and a plant cell.

28. The host cell of claim 27, wherein the cell is a yeast cell.

29. The host cell of claim 28, wherein the cell is a Saccharomyces cerevisiae.

30. A method for producing one or more steviol glycosides comprising:
culturing a population of host cells of claim 15, in a medium with a carbon source under conditions suitable for making one or more steviol glycosides to yield a culture broth; and
recovering the one or more steviol glycosides from the culture broth.

31. The method of claim 30, wherein the one or more steviol glycosides is RebM.

32. A fermentation composition comprising:
a host cell comprising a nucleic acid encoding a kaurenoic acid hydroxylase of claim 15; and
one or more steviol glycosides produced by the host cell.

33-49. (canceled)


SEQUENCE LISTING (23 sequences)

SEQ ID NO: 1 - protein, 523 aa, Rubus occidentalis
MEVTVGSWVALSLVFVSIIVGWAWSVLDWVWLKPKKLERCLREQGLKGNSYWFLYGDMKENSILLKQAKSKPMNLSTSHDIAPQVIPFVDQTVKVYGKNSFDWIGPIPRVNIMNPEELKDVFTKYDDFIKPISNPLFKLLATGLANYEGEKWAKHRRIINPTFHSEKLKRMLPSFHQSCTEMIKEWESLVSKEGSSCELDVWPFLENMTADVISRTAFGTSYKKGRKIFELLREQAIYATKAIQSFYIPGWRFLPTKMNKRMKEINKEIKGLIKGIIIKREHTIKAGEETKDDLLGALMESNLKDIREHGKNNKNFGMSIEDVIEECKLFYFAGQETTSVLLVWTMVLLGQNQNWQDRARQEILQVFGSNKPDFDGLTHLKVVTMILLEVLRLYPAVIELPRTIHKKTQLGKFSLPEGVEVRLPTLLIHHDKELWGDDANEFKPERFSEGVSKATKSRLSFFPFGGGPRICIGQNFAMMEAKLALVLILQHFTFELSPSYAHAPSYRITLQPQYGVPIILHRR

SEQ ID NO: 2 - DNA, 1571 bp, Rubus occidentalis
  source 1..1571: mol_type = other DNA
atggaagtaaccgttggatcttgggtagctttgtccttagtcttcgtttctattatcgtcggttgggcttggtccgttttagattgggtctggttgaaaccaaagaagttagaaagatgtttgagagaacaaggtttaaagggtaactcttactggttcttgtatggtgacatgaaagagaactctattttgttgaagcaagctaagtctaagccaatgaacttatctacctctcacgacatcgccccacaagttattccatttgtcgaccaaactgtcaaggtctacggtaagaactctttcgattggatcggtcctattccaagagtcaatatcatgaacccagaagaattgaaggatgttttcaccaagtacgatgacttcatcaagccaatttctaaccctttgttcaagttgttggctaccggtttggctaattacgaaggtgagaagtgggctaagcacagacgtattatcaacccaactttccattctgagaagttgaaaagaatgttgccatccttccaccaatcttgtactgaaatgatcaaggaatgggaatctttggtttctaaggaaggttcttcttgtgagttagacgtctggccattcttagaaaacatgaccgctgacgttatttctagaactgctttcggtacttcttacaagaagggtagaaagattttcgaattgttgagagaacaagctatttacgccaccaaggctatccaatctttttacattccaggttggcgttttttgcctactaaaatgaacaagagaatgaaggaaatcaacaaggagatcaagggtttgattaagggtatcatcatcaaaagagaacacactatcaaggctggtgaagaaactaaggatgacttgttaggtgctttgatggaatctaacttgaaggacattagagaacacggtaagaacaacaagaacttcggtatgtctatcgaagacgttatcgaagagtgtaagttgttctactttgctggtcaagaaactacttctgttttgttagtttggaccatggttttgttgggtcaaaatcaaaactggcaagatagagctagacaagaaatcttgcaagtttttggttctaataagccagacttcgatggtttgactcacttgaaagttgtcaccatgattttattggaagtcttgagattgtacccagctgttatcgaattgccaagaaccattcacaagaagactcaattgggtaaattctctttacctgaaggtgttgaagttagattgccaactttgttaatccaccatgataaggaattgtggggtgatgacgctaacgaattcaagccagaacgtttctctgaaggtgtttctaaggctaccaaatccagattgtccttttttcctttcggtggtggtcctagaatctgtattggtcaaaactttgctatgatggaagctaaattggctttggttttgattttgcaacacttcactttcgaattgtccccttcctacgcccatgctccatcctacagaattaccttacaacctcaatatggtgtccctattatcttgcaccgtcgtta

SEQ ID NO: 3 - protein, 523 aa, Rubus suavissimus
MEVTVASSVALSLVFISIVVRWAWSVVNWVWFKPKKLERFLREQGLKGNSYRFLYGDMKENSILLKQARSKPMNLSTSHDIAPQVTPFVDQTVKAYGKNSFNWVGPIPRVNIMNPEDLKDVLTKNVDFVKPISNPLIKLLATGIAIYEGEKWTKHRRIINPTFHSERLKRMLPSFHQSCNEMVKEWESLVSKEGSSCELDVWPFLENMSADVISRTAFGTSYKKGQKIFELLREQVIYVTKGFQSFYIPGWRFLPTKMNKRMNEINEEIKGLIRGIIIDREQIIKAGEETNDDLLGALMESNLKDIREHGKNNKNVGMSIEDVIQECKLFYFAGQETTSVLLAWTMVLLGQNQNWQDRARQEVLQVFGSSKPDFDGLAHLKVVTMILLEVLRLYPPVIELIRTIHKKTQLGKLSLPEGVEVRLPTLLIHHDKELWGDDANQFNPERFSEGVSKATKNRLSFFPFGAGPRICIGQNFSMMEAKLALALILQHFTFELSPSHAHAPSHRITLQPQYGVRIILHRR

SEQ ID NO: 4 - protein, 525 aa, Arabidopsis thaliana
MESLVVHTVNAIWCIVIVGIFSVGYHVYGRAVVEQWRMRRSLKLQGVKGPPPSIFNGNVSEMQRIQSEAKHCSGDNIISHDYSSSLFPHFDHWRKQYGRIYTYSTGLKQHLYINHPEMVKELSQTNTLNLGRITHITKRLNPILGNGIITSNGPHWAHQRRIIAYEFTHDKIKGMVGLMVESAMPMLNKWEEMVKRGGEMGCDIRVDEDLKDVSADVIAKACFGSSFSKGKAIFSMIRDLLTAITKRSVLFRFNGFTDMVFGSKKHGDVDIDALEMELESSIWETVKEREIECKDTHKKDLMQLILEGAMRSCDGNLWDKSAYRRFVVDNCKSIYFAGHDSTAVSVSWCLMLLALNPSWQVKIRDEILSSCKNGIPDAESIPNLKTVTMVIQETMRLYPPAPIVGREASKDIRLGDLVVPKGVCIWTLIPALHRDPEIWGPDANDFKPERFSEGISKACKYPQSYIPFGLGPRTCVGKNFGMMEVKVLVSLIVSKFSFTLSPTYQHSPSHKLLVEPQHGVVIRVV

SEQ ID NO: 5 - protein, 500 aa, Stevia rebaudiana
MEASYLYISILLLLASYLFTTQLRRKSANLPPTVFPSIPIIGHLYLLKKPLYRTLAKIAAKYGPILQLQLGYRRVLVISSPSAAEECFTNNDVIFANRPKTLFGKIVGGTSLGSLSYGDQWRNLRRVASIEILSVHRLNEFHDIRVDENRLLIRKLRSSSSPVTLITVFYALTLNVIMRMISGKRYFDSGDRELEEEGKRFREILDETLLLAGASNVGDYLPILNWLGVKSLEKKLIALQKKRDDFFQGLIEQVRKSRGAKVGKGRKTMIELLLSLQESEPEYYTDAMIRSFVLGLLAAGSDTSAGTMEWAMSLLVNHPHVLKKAQAEIDRVIGNNRLIDESDIGNIPYIGCIINETLRLYPAGPLLFPHESSADCVISGYNIPRGTMLIVNQWAIHHDPKVWDDPETFKPERFQGLEGTRDGFKLMPFGSGRRGCPGEGLAIRLLGMTLGSVIQCFDWERVGDEMVDMTEGLGVTLPKAVPLVAKCKPRSEMTNLLSEL

SEQ ID NO: 6 - protein, 513 aa, Stevia rebaudiana
MDAVTGLLTVPATAITIGGTAVALAVALIFWYLKSYTSARRSQSNHLPRVPEVPGVPLLGNLLQLKEKKPYMTFTRWAATYGPIYSIKTGATSMVVVSSNEIAKEALVTRFQSISTRNLSKALKVLTADKTMVAMSDYDDYHKTVKRHILTAVLGPNAQKKHRIHRDIMMDNISTQLHEFVKNNPEQEEVDLRKIFQSELFGLAMRQALGKDVESLYVEDLKITMNRDEIFQVLVVDPMMGAIDVDWRDFFPYLKWVPNKKFENTIQQMYIRREAVMKSLIKEHKKRIASGEKLNSYIDYLLSEAQTLTDQQLLMSLWEPIIESSDTTMVTTEWAMYELAKNPKLQDRLYRDIKSVCGSEKITEEHLSQLPYITAIFHETLRRHSPVPIIPLRHVHEDTVLGGYHVPAGTELAVNIYGCNMDKNVWENPEEWNPERFMKENETIDFQKTMAFGGGKRVCAGSLQALLTASIGIGRMVQEFEWKLKDMTQEEVNTIGLTTQMLRPLRAIIKPRI

SEQ ID NO: 7 - protein, 320 aa, Blakeslea trispora
MLTSSKSIESFPKNVQPYGKHYQNGLEPVGKSQEDILLEPFHYLCSNPGKDVRTKMIEAFNAWLKVPKDDLIVITRVIEMLHSASLLIDDVEDDSVLRRGVPAAHHIYGTPQTINCANYVYFLALKEIAKLNKPNMITIYTDELINLHRGQGMELFWRDTLTCPTEKEFLDMVNDKTGGLLRLAVKLMQEASQSGTDYTGLVSKIGIHFQVRDDYMNLQSKNYADNKGFCEDLTEGKFSFPIIHSIRSDPSNRQLLNILKQRSSSIELKQFALQLLENTNTFQYCRDFLRVLEKEAREEIKLLGGNIMLEKIMDVLSVNE

SEQ ID NO: 8 - protein, 736 aa, Oryza sativa
MEHARPPQGGDDDVAASTSELPYMIESIKSKLRAARNSLGETTVSAYDTAWIALVNRLDGGGERSPQFPEAIDWIARNQLPDGSWGDAGMFIVQDRLINTLGCVVALATWGVHEEQRARGLAYIQDNLWRLGEDDEEWMMVGFEITFPVLLEKAKNLGLDINYDDPALQDIYAKRQLKLAKIPREALHARPTTLLHSLEGMENLDWERLLQFKCPAGSLHSSPAASAYALSETGDKELLEYLETAINNFDGGAPCTYPVDNFDRLWSVDRLRRLGISRYFTSEIEEYLEYAYRHLSPDGMSYGGLCPVKDIDDTAMAFRLLRLHGYNVSSSVFNHFEKDGEYFCFAGQSSQSLTAMYNSYRASQIVFPGDDDGLEQLRAYCRAFLEERRATGNLRDKWVIANGLPSEVEYALDFPWKASLPRVETRVYLEQYGASEDAWIGKGLYRMTLVNNDLYLEAAKADFTNFQRLSRLEWLSLKRWYIRNNLQAHGVTEQSVLRAYFLAAANIFEPNRAAERLGWARTAILAEAIASHLRQYSANGAADGMTERLISGLASHDWDWRESNDSAARSLLYALDELIDLHAFGNASDSLREAWKQWLMSWTNESQGSTGGDTALLLVRTIEICSGRHGSAEQSLKNSEDYARLEQIASSMCSKLATKILAQNGGSMDNVEGIDQEVDVEMKELIQRVYGSSSNDVSSVTRQTFLDVVKSFCYVAHCSPETIDGHISKVLFEDVN

SEQ ID NO: 9 - protein, 757 aa, Picea glauca
MKREQYTILNEKESMAEELILRIKRMFSEIENTQTSASAYDTAWVAMVPSLDSSQQPQFPQCLSWIIDNQLLDGSWGIPYLIIKDRLCHTLACVIALRKWNAGNQNVETGLRFLRENIEGIVHEDEYTPIGFQIIFPAMLEEARGLGLELPYDLTPIKLMLTHREKIMKGKAIDHMHEYDSSLIYTVEGIHKIVDWNKVLKHQNKDGSLFNSPSATACALMHTRKSNCLEYLSSMLQKLGNGVPSVYPINLYARISMIDRLQRLGLARHFRNEIIHALDDIYRYWMQRETSREGKSLTPDIVSTSIAFMLLRLHGYDVPADVFCCYDLHSIEQSGEAVTAMLSLYRASQIMFPGETILEEIKTVSRKYLDKRKENGGIYDHNIVMKDLRGEVEYALSVPWYASLERIENRRYIDQYGVNDTWIAKTSYKIPCISNDLFLALAKQDYNICQAIQQKELRELERWFADNKFSHLNFARQKLIYCYFSAAATLFSPELSAARVVWAKNGVITTVVDDFFDVGGSSEEIHSFVEAVRVWDEAATDGLSENVQILFSALYNTVDEIVQQAFVFQGRDISIHLREIWYRLVNSMMTEAQWARTHCLPSMHEYMENAEPSIALEPIVLSSLYFVGPKLSEEIICHPEYYNLMHLLNICGRLLNDIQGCKREAHQGKLNSVTLYMEENSGTTMEDAIVYLRKTIDESRQLLLKEVLRPSIVPRECKQLHWNMMRILQLFYLKNDGFTSPTEMLGYVNAVIVDPIL

SEQ ID NO: 10 - protein, 499 aa, Pisum Sativum
MDTLTLSLGFLSLFLFLFLLKRSTHKHSKLSHVPVVPGLPVIGNLLQLKEKKPHKTFTKMAQKYGPIFSIKAGSSKIIVLNTAHLAKEAMVTRYSSISKRKLSTALTILTSDKCMVAMSDYNDFHKMVKKHILASVLGANAQKRLRFHREVMMENMSSKFNEHVKTLSDSAVDFRKIFVSELFGLALKQALGSDIESIYVEGLTATLSREDLYNTLVVDFMEGAIEVDWRDFFPYLKWIPNKSFEKKIRRVDRQRKIIMKALINEQKKRLTSGKELDCYYDYLVSEAKEVTEEQMIMLLWEPIIETSDTTLVTTEWAMYELAKDKNRQDRLYEELLNVCGHEKVTDEELSKLPYLGAVFHETLRKHSPVPIVPLRYVDEDTELGGYHIPAGSEIAINIYGCNMDSNLWENPDQWIPERFLDEKYAQADLYKTMAFGGGKRVCAGSLQAMLIACTAIGRLVQEFEWELGHGEEENVDTMGLTTHRLHPLQVKLKPRNRIY

SEQ ID NO: 11 - protein, 711 aa, Arabidopsis thaliana
MSSSSSSSTSMIDLMAAIIKGEPVIVSDPANASAYESVAAELSSMLIENRQFAMIVTTSIAVLIGCIVMLVWRRSGSGNSKRVEPLKPLVIKPREEEIDDGRKKVTIFFGTQTGTAEGFAKALGEEAKARYEKTRFKIVDLDDYAADDDEYEEKLKKEDVAFFFLATYGDGEPTDNAARFYKWFTEGNDRGEWLKNLKYGVFGLGNRQYEHFNKVAKVVDDILVEQGAQRLVQVGLGDDDQCIEDDFTAWREALWPELDTILREEGDTAVATPYTAAVLEYRVSIHDSEDAKFNDINMANGNGYTVFDAQHPYKANVAVKRELHTPESDRSCIHLEFDIAGSGLTYETGDHVGVLCDNLSETVDEALRLLDMSPDTYFSLHAEKEDGTPISSSLPPPFPPCNLRTALTRYACLLSSPKKSALVALAAHASDPTEAERLKHLASPAGKDEYSKWVVESQRSLLEVMAEFPSAKPPLGVFFAGVAPRLQPRFYSISSSPKIAETRIHVTCALVYEKMPTGRIHKGVCSTWMKNAVPYEKSENCSSAPIFVRQSNFKLPSDSKVPIIMIGPGTGLAPFRGFLQERLALVESGVELGPSVLFFGCRNRRMDFIYEEELQRFVESGALAELSVAFSREGPTKEYVQHKMMDKASDIWNMISQGAYLYVCGDAKGMARDVHRSLHTIAQEQGSMDSTKAEGFVKNLQTSGRYLRDVW

SEQ ID NO: 12 - protein, 481 aa, Stevia rebaudiana
MDAMATTEKKPHVIFIPFPAQSHIKAMLKLAQLLHHKGLQITFVNTDFIHNQFLESSGPHCLDGAPGFRFETIPDGVSHSPEASIPIRESLLRSIETNFLDRFIDLVTKLPDPPTCIISDGFLSVFTIDAAKKLGIPVMMYWTLAACGFMGFYHIHSLIEKGFAPLKDASYLTNGYLDTVIDWVPGMEGIRLKDFPLDWSTDLNDKVLMFTTEAPQRSHKVSHHIFHTFDELEPSIIKTLSLRYNHIYTIGPLQLLLDQIPEEKKQTGITSLHGYSLVKEEPECFQWLQSKEPNSVVYVNFGSTTVMSLEDMTEFGWGLANSNHYFLWIIRSNLVIGENAVLPPELEEHIKKRGFIASWCSQEKVLKHPSVGGFLTHCGWGSTIESLSAGVPMICWPYSWDQLTNCRYICKEWEVGLEMGTKVKRDEVKRLVQELMGEGGHKMRNKAKDWKEKARIAIAPNGSSSLNIDKMVKEITVLARN

SEQ ID NO: 13 - protein, 460 aa, Stevia rebaudiana
MAEQQKIKKSPHVLLIPFPLQGHINPFIQFGKRLISKGVKTTLVTTIHTLNSTLNHSNTTTTSIEIQAISDGCDEGGFMSAGESYLETFKQVGSKSLADLIKKLQSEGTTIDAIIYDSMTEWVLDVAIEFGIDGGSFFTQACVVNSLYYHVHKGLISLPLGETVSVPGFPVLQRWETPLILQNHEQIQSPWSQMLFGQFANIDQARWVFTNSFYKLEEEVIEWTRKIWNLKVIGPTLPSMYLDKRLDDDKDNGFNLYKANHHECMNWLDDKPKESVVYVAFGSLVKHGPEQVEEITRALIDSDVNFLWVIKHKEEGKLPENLSEVIKTGKGLIVAWCKQLDVLAHESVGCFVTHCGFNSTLEAISLGVPVVAMPQFSDQTTNAKLLDEILGVGVRVKADENGIVRRGNLASCIKMIMEEERGVIIRKNAVKWKDLAKVAVHEGGSSDNDIVEFVSELIKA

SEQ ID NO: 14 - protein, 485 aa, Stevia rebaudiana
MYNVTYHQNSKAMATSDSIVDDRKQLHVATFPWLAFGHILPYLQLSKLIAEKGHKVSFLSTTRNIQRLSSHISPLINVVQLTLPRVQELPEDAEATTDVHPEDIPYLKKASDGLQPEVTRFLEQHSPDWIIYDYTHYWLPSIAASLGISRAHFSVTTPWAIAYMGPSADAMINGSDGRTTVEDLTTPPKWFPFPTKVCWRKHDLARLVPYKAPGISDGYRMGLVLKGSDCLLSKCYHEFGTQWLPLLETLHQVPVVPVGLLPPEIPGDEKDETWVSIKKWLDGKQKGSVVYVALGSEVLVSQTEVVELALGLELSGLPFVWAYRKPKGPAKSDSVELPDGFVERTRDRGLVWTSWAPQLRILSHESVCGFLTHCGSGSIVEGLMFGHPLIMLPIFGDQPLNARLLEDKQVGIEIPRNEEDGCLTKESVARSLRSVVVEKEGEIYKANARELSKIYNDTKVEKEYVSQFVDYLEKNARAVAIDHES

SEQ ID NO: 15 - protein, 458 aa, Stevia rebaudiana
MENKTETTVRRRRRIILFPVPFQGHINPILQLANVLYSKGFSITIFHTNFNKPKTSNYPHFTFRFILDNDPQDERISNLPTHGPLAGMRIPIINEHGADELRRELELLMLASEEDEEVSCLITDALWYFAQSVADSLNLRRLVLMTSSLFNFHAHVSLPQFDELGYLDPDDKTRLEEQASGFPMLKVKDIKSAYSNWQILKEILGKMIKQTKASSGVIWNSFKELEESELETVIREIPAPSFLIPLPKHLTASSSSLLDHDRTVFQWLDQQPPSSVLYVSFGSTSEVDEKDFLEIARGLVDSKQSFLWVVRPGFVKGSTWVEPLPDGFLGERGRIVKWVPQQEVLAHGAIGAFWTHSGWNSTLESVCEGVPMIFSDFGLDQPLNARYMSDVLKVGVYLENGWERGEIANAIRRVMVDEEGEYIRQNARVLKQKADVSLMKGGSSYESLESLVSYISSL

SEQ ID NO: 16 - protein, 436 aa, Setaria italica
MDASDSSPLHIVIFPWLAFGHMLASLELAERLAARGHRVSFVSTPRNISRLRPVPPALAPLIDFVALPLPRVDGLPDGAEATSDIPPGKTELHLKALDGLAAPFAAFLDAACADGSTNKVDWLFLDNFQYWAAAAAADHKIPCALNLTFAASTSAEYGVPRVEPPVDGSTASILQRFVLTLEKCQFVIQRACFELEPEPLPLLSDIFGKPVIPYGLVPPCPPAEGHKREHGNAALSWLDKQQPESVLFIALGSEPPVTVEQLHEIALGLELAGTTFLWALKKPNGLLLEADGDILPPGFEERTRDRGLVAMGWVPQPIILAHSSVGAFLTHGGWASTIEGVMSGHPMLFLTFLDEQRINAQLIERKKAGLRVPRREKDGSYDRQGIAGAIRAVMCEEESKSVFAANAKKMQEIVSDRNCQEKYIDELIQRLGSFEK

SEQ ID NO: 17 - protein, 564 aa, synthetic construct
MQSTTSVKLSPFDLMTALLNGKVSFDTSNTSDTNIPLAVFMENRELLMILTTSVAVLIGCVVVLVWVLDWVWLKPKKLERCLREQGLKGNSYWFLYGDMKENSILLKQAKSKPMNLSTSHDIAPQVIPFVDQTVKVYGKNSFDWIGPIPRVNIMNPEELKDVFTKYDDFIKPISNPLFKLLATGLATYEGEKWAKHRRIINPTFHSEKLKRMLPSFHQSCTEMIKEWESLVSKEGSSCELDVWPFLENMTADVISRTAFGTSYKKGRKIFELLREQAIYATKAIQSFYIPGWRFLPTKMNKRMKEINKEIKGLIKGIIIKREHTIKAGEETKDDLLGYLMESNLKDIREHGKNNKNFGMSIEDVIEECKLFYFAGQETTSVLLVWTMVLLGQNQNWQDRARQEILQVFGSNKPDFDGLTHLKVVTMILLEVLRLYPAVIELPRTIHKKTQLGKFSLPEGVEVRLPTLLIHHDKELWGDDANEFKPERFSEGVSKATKSRLSFFPFGAGPRICIGQNFAMMEAKLALVLILQHFTFELSPSYAHAPSYRITLQPQYGVPIILHRR

SEQ ID NO: 18 - protein, 564 aa, synthetic construct
MQSTTSVKLSPFDLMTALLNGKVSFDTSNTSDTNIPLAVFMENRELLMILTTSVAVLIGCVVVLVWVLDWVWLKPKKLERCLREQGLKGNSYTFLYGDMKENSILLKQAKSKPMNLSTSHDIAPQVIPFVDQTVKVYGKNSFDWIGPIPRVNIMNPEELKDVFTKYDDFIKPISNPLFKLLATGLATYEGEKWAKHRRIINPTFHSEKLKRMLPSFHQSCTEMIKEWESLVSKEGSSCELDVWPFLENMTADVISRTAFGTSYKKGRKIFELLREQAIYATKAIQSFYIPGWRFLPTKMNKRMKEINKEIKGLIKGIIIKREHTIKAGEETKDDLLGYLMESNLKDIREHGKNNKNFGMSIEDVIEECKLFYFAGQETTSVLLVWTMVLLGQNQNWQDRARQEILQVFGSNKPDFDGLTHLKVVTMILLEVLRLYPAVIELPRTIHKKTQLGKFSLPEGVEVRLPTLLIHHDKELWGDDANEFKPERFSEGVSKATKSRLSFFPFGGGPRICIGQNFAMMEAKLALVLILQHFTFELSPSYAHAPSYRITLQPQYGVPIILHRR

SEQ ID NO: 19 - protein, 564 aa, synthetic construct
MQSTTSVKLSPFDLMTALLNGKVSFDTSNTSDTNIPLAVFMENRELLMILTTSVAVLIGCVVVLVWVLDWVWLKPKKLERCLREQGLKGNSYWFLYGDMKENSILLKQAKSKPMNLSTSHDIAPQVIPFVDQTVKVYGKNSFDWIGPIPRVNIMNPEELKDVFTKYDDFIKPISNPLFKLLAGGLATYEGEKWAKHRRIINPTFHSEKLKRMLPSFHQSCTEMIKEWESLVSKEGSSCELDVWPFLENMTADVISRTAFGTSYKKGRKIFELLREQAIYATKAIQSFYIPGWRFLPTKMNKRMKEINKEIKGLIKGIIIKREHTIKAGEETKDDLLGYLMESNLKDIREHGKNNKNFGMSIEDVIEECKLFYFAGQETTSVLLVWTMVLLGQNQNWQDRARQEILQVFGSNKPDFDGLTHLKVVTMILLEVLRLYPAVIELPRTIHKKTQLGKFSLPEGVEVRLPTLLIHHDKELWGDDANEFKPERFSEGVSKATKSRLSFFPFGAGPRICIGQNFAMMEAKLALVLILQHFTFELSPSYAHAPSYRITLQPQYGVPIILHRR

SEQ ID NO: 20 - protein, 564 aa, synthetic construct
MQSTTSVKLSPFDLMTALLNGKVSFDTSNTSDTNIPLAVFMENRELLMILTTSVAVLIGCVVVLVWVLDWVWLKPKKLERCLREQGLKGNSYTFLYGDMKENSILLKQAKSKPMNLSTSHDIAPQVIPFVDQTVKVYGKNSFDWIGPIPRVNIMNPEELKDVFTKYDDFIKPISNPLFKLLAGGLATYEGEKWAKHRRIINPTFHSEKLKRMLPSFHQSCTEMIKEWESLVSKEGSSCELDVWPFLENMTADVISRTAFGTSYKKGRKIFELLREQAIYATKAIQSFYIPGWRFLPTKMNKRMKEINKEIKGLIKGIIIKREHTIKAGEETKDDLLGYLMESNLKDIREHGKNNKNFGMSIEDVIEECKLFYFAGQETTSVLLVWTMVLLGQNQNWQDRARQEILQVFGSNKPDFDGLTHLKVVTMILLEVLRLYPAVIELPRTIHKKTQLGKFSLPEGVEVRLPTLLIHHDKELWGDDANEFKPERFSEGVSKATKSRLSFFPFGGGPRICIGQNFAMMEAKLALVLILQHFTFELSPSYAHAPSYRITLQPQYGVPIILHRR

SEQ ID NO: 21 - protein, 564 aa, synthetic construct
MQSTTSVKLSPFDLMTALLNGKVSFDTSNTSDTNIPLAVFMENRELLMILTTSVAVLIGCVVVLVWVLDWVWLKPKKLERCLREQGLKGNSYTFLYGDMKENSILLKQAKSKPMNLSTSHDIAPQVIPFVDQTVKVYGKNSFDWIGPIPRVNIMNPEELKDVFTKYDDFIKPISNPLFKLLAGGLATYEGEKWAKHRRIINPTFHSEKLKRMLPSFHQSCTEMIKEWESLVSKEGSSCELDVWPFLENMTADVISRTAFGTSYKKGRKIFELLREQAIYATKAIQSFYIPGWRFLPTKMNKRMKEINKEIKGLIKGIIIKREHTIKAGEETKDDLLGYLMESNLKDIREHGKNNKNFGMSIEDVIEECKLFYFAGQETTSVLLVWTMVLLGQNQNWQDRARQEILQVFGSNKPDFDGLTHLKVVTMILLEVLRLYPAVIELPRTIHKKTQLGKFSLPEGVEVRLPTLLIHHDKELWGDDANEFKPERFSEGVSKATKSRLSFFPFGAGPRICIGQNFAMMEAKLALVLILQHFTFELSPSYAHAPSYRITLQPQYGVPIILHRR

SEQ ID NO: 22 - protein, 66 aa, synthetic construct
MQSTTSVKLSPFDLMTALLNGKVSFDTSNTSDTNIPLAVFMENRELLMILTTSVAVLIGCVVVLVW

SEQ ID NO: 23 - protein, 462 aa, Oryza sativa
MDSGYSSSYAAAAGMHVVICPWLAFGHLLPCLDLAQRLASRGHRVSFVSTPRNISRLPPVRPALAPLVAFVALPLPRVEGLPDGAESTNDVPHDRPDMVELHRRAFDGLAAPFSEFLGTACADWVIVDVFHHWAAAAALEHKVPCAMMLLGSAHMIASIADRRLERAETESPAAAGQGRPAAAPTFEVARMKLIRTKGSSGMSLAERFSLTLSRSSLVVGRSCVEFEPETVPLLSTLRGKPITFLGLMPPLHEGRREDGEDATVRWLDAQPAKSVVYVALGSEVPLGVEKVHELALGLELAGTRFLWALRKPTGVSDADLLPAGFEERTRGRGVVATRWVPQMSILAHAAVGAFLTHCGWNSTIEGLMFGHPLIMLPIFGDQGPNARLIEAKNAGLQVARNDGDGSFDREGVAAAIRAVAVEEESSKVFQAKAKKLQEIVADMACHERYIDGFIQQLRSYKD